(19) United States
(12) Patent Application Publication
(10) Pub. No.: US 2023/0309796 A1
(43) Pub. Date: Oct. 5, 2023

(54) ENDOSCOPE SYSTEM

(71) Applicant: AdaptivEndo LLC, Louisville, KY (US)

(72) Inventors: Hershel E. Fancher, Georgetown, IN (US); Mark A. Griffin, Louisville, KY (US); Michael W. Goldenbogen, Xenia, OH (US); Gregory R. Furnish, Louisville, KY (US); Nicholas Canfield, Louisville, KY (US); Galen R. Powers, Louisville, KY (US); Trevor E. Powers, Louisville, KY (US); Sumner E. Adams, Louisville, KY (US)

(21) Appl. No.: 18/322,004

(22) Filed: May 23, 2023

Related U.S. Application Data
(63) Continuation of application No. PCT/US2021/060615, filed on Nov. 23, 2021.
(60) Provisional application No. 63/117,003, filed on Nov. 23, 2020.
(60) Provisional application No. 63/119,759, filed on Dec. 1, 2020.
(60) Provisional application No. 63/122,167, filed on Dec. 7, 2020.
(60) Provisional application No. 63/122,171, filed on Dec. 7, 2020.
(60) Provisional application No. 63/146,526, filed on Feb. 5, 2021.
(60) Provisional application No. 63/185,734, filed on May 7, 2021.

(51) Int. Cl. A61B 1/00; A61B 1/005

(52) U.S. Cl. CPC A61B 1/00105; A61B 1/0052; A61B 1/0057; A61B 1/00103; A61B 1/00098; A61B 1/05

(57) ABSTRACT

Arrangements comprising an endoscope system having an endoscope including a reusable hand-piece, a separable single-use shaft assembly, a single-use cartridge and a console are disclosed. Endoscope assemblies having articulation knobs attached by friction fit, multi-stage rotational distance limiters, and/or elevators are disclosed. Components of same, packaging systems, and methods are also provided.

### CROSS-REFERENCE TO RELATED APPLICATION

This application is a continuation of PCT Application No. US2021/060615 filed Nov. 23, 2021, which is hereby incorporated by reference. PCT Application No. US2021/060615 claims the benefit of U.S. Provisional Application No. 63/117,003 filed Nov. 23, 2020, U.S. Provisional Application No. 63/119,759 filed Dec. 1, 2020, U.S. Provisional Application No. 63/122,167 filed Dec. 7, 2020, U.S. Provisional Application No. 63/122,171 filed Dec. 7, 2020, U.S. Provisional Application No. 63/146,526 filed Feb. 5, 2021, and U.S. Provisional Application No. 63/185,734 filed May 7, 2021, which are hereby incorporated by reference.

### BACKGROUND

This disclosure generally relates to endoscopes, including endoscopes used in gastroenterological applications.

Endoscopes are used in a wide variety of medical procedures to visualize internal cavities or potential spaces within the human body during either diagnostic or therapeutic procedures.

One factor affecting endoscope design and use is infectious disease transmission. Although infectious disease transmission has always been a risk with reusable endoscopes, this risk has increased with the advent of antibiotic resistant bacteria. Once transmission of these bacteria has occurred between patients, antibiotic resistance makes it difficult to treat these infections. Many of these bacteria, including staphylococcus, have the ability to form a protective outer barrier, frequently referred to as a bio-film, which protects the infectious bacteria from during cleaning procedures.

Another recent development that increases the risk of infectious disease transmission is an ever-increasing number of diagnostic procedures that require the use of smaller and more complicated instruments and longer working lumens and distal manipulators, e.g. elevators, associated with the endoscope distal articulating shaft. Together, the increasing prevalence of difficult to treat infectious diseases and more complex, difficult-to-clean endoscopes make the sterilization and re-use of endoscopes an increasingly risky activity.

Reducing or limiting the potential for infectious disease transmission is desirable. Accordingly, new endoscope designs are desired.

Thus, there is a need for improvement in this field.

### SUMMARY

The present disclosure pertains generally to endoscopes. It is understood that many of the aspects disclosed are applicable in other technical areas, but especially in other medical devices.

The present disclosure provides an endoscope assembly comprising a reusable hand-piece and a single-use shaft assembly. The disclosure may include an endoscope assembly, cartridge and a console. More particularly, the endoscope assembly may include the reusable hand-piece attached to the single-use shaft assembly. The reusable hand-piece may be detached from the single-use shaft assembly.

The reusable hand-piece is selectively attachable and detachable from the single-use shaft assembly such that the reusable hand-piece may be used serially with a number of single-use shaft assemblies while the single-use shaft assembly, including the cartridges, can be discarded and/or reconditioned after a single use. The reusable hand-piece and/or single-use shaft assemblies may include a lock for selectively locking the reusable hand-piece and single-use shaft assembly to one another. A latch handle (e.g., a lever) for actuating the latching mechanism connecting the reusable hand-piece to the single-use shaft assembly may be positioned on the reusable hand-piece and/or the single-use shaft assembly. Additional locking mechanisms such as magnets may be used in the alternative or concurrently.

The reusable hand-piece can include a hand-piece housing supporting an articulation knob and brake assembly; control switches for operating light(s), such as light emitting diodes, and/or flush and/or aspiration fluid paths; one or more navigation switches for controlling a console communicating with the endoscope assembly; and/or an electrical connector for electrically coupling the reusable hand-piece to the single-use shaft assembly.

The single-use shaft assembly can include an insertion tube for insertion within the body of a patient, a tubing and electrical connector connected to a console, an access port for selectively accessing a lumen of the single-use shaft assembly (e.g., for taking a sample such as a biopsy) and/or for delivering a fluid through a lumen of the single-use shaft assembly, and/or an electrical connector for electrically coupling the single-use shaft assembly to the reusable hand-piece.

A tubing and/or electrical connector, sometimes referred to as an “umbilical”, extends from the endoscope assembly to a cartridge that is connectable to a console suitable for use with the endoscope assembly. The tubing and/or electrical connector can extend from the single-use shaft assembly and/or from the reusable hand-piece. Often, the electrical connector extends through the tubing along with one or more fluid paths.

A console may provide electrical power, fluids and/or fluid pressure, vacuum pressure, and/or send and/or receive electrical signals with the endoscope assembly. The console may include a monitor for visualizing signals received from the endoscope assembly such as a video feed from a camera at the distal end of the single-use shaft assembly.

The single-use shaft assembly can include one or more pulley assemblies. Pulley assemblies can include at least one pulley arranged to receive an end of an articulation shaft. Preferably, rotation of the articulation knob associated with the articulation shaft rotates the at least one pulley. Preferably, the at least one pulley and end of the articulation shaft have a geometry (e.g., a mating feature) for rotationally coupling the pulley and articulation shaft to one another. Preferably the mating feature does not rely solely on friction between the articulation shaft and pulley. Preferably the mating features include a geometric interference between the articulation shaft and pulley. The pulley assemblies may include a circular pulley and/or a non-circular pulley (e.g., a cam).

Associated with the at least one pulley is an articulation wire or a pair of articulation pull wires (e.g., two separate wires or one continuous wire looped over the pulley and having wire segments extending from opposing sides of the pulley) configured to bend a distal shaft section of the single-use shaft assembly upon actuation. The articulation wires may be fixed (e.g., tied or fixed by anchors (not illustrated)) to the pulley and/or extend around at least a portion of the pulley (e.g., loop around the pulley). Preferably, the one or more pulley assemblies include two pulleys (one for each articulation wire) and articulation wires (at least one for each pulley). In the illustrated example, each pulley includes one groove (two total) for the articulation wires. Alternatively, each pulley may include two grooves (four total) with only one wire per groove to reduce the chance the wires overlap. Preferably, the articulation wires are configured to bend the distal shaft section in orthogonal planes.

The one or more pulleys and associated one or more articulation wires may be contained within a pulley housing. The pulley housing may be constructed of housing portions, such as cover pieces and inner pieces. The housing portions may cooperate to at least partially enclose the one or more pulleys.

The pulley housing may be constructed in a sandwich-like fashion with a pair of cover pieces on the outside/ends and a pair of inner pieces coupled on one side to one cover piece each and on the other side to each other. Such housing portions may cooperate to enclose the pulley and associated articulation wire(s). Preferably the housing portions resist the articulation wire(s) from separating from the pulley(s). The pulley housing can define openings for receiving articulation shafts and/or a brake shaft of the articulation control assembly.

Preferably, the housing portions do not contact one another along a purely linear butt joint. For example, the inner pieces and/or cover pieces may contact along a rectilinear path. In some aspects, the inner pieces and cover pieces are constructed with a castellation along the joint between the two pieces and down the length of the pieces so that the castellation on the inner piece matches with and couples to the castellation on the cover piece. Advantageously, avoiding a purely linear butt joint can assist in preventing wires from escaping between portions of the pulley assembly. In other examples, an intersection between housing portions may be linear, curved, curvilinear, rectilinear, or some other desired arrangement.

In addition, the pulley assembly may include an articulation coil termination block that receives one or more articulation coils wrapping around respective articulation pull wires connected to the pulleys. The articulation coil termination block is adjustable to remove articulation pull wire slack. In other words, the articulation coil termination block is adjustable along the longitudinal axis of the endoscope assembly to control the articulation pull wire tension.

The present disclosure includes a “two stage articulation limit.” The two stage rotational stop assembly allows the articulation knobs to rotate further than with a single stage stop. The two stage rotational stop assembly may be completely enclosed within the pulley housing. The two stage rotational stop assembly may include two pulleys and two rotational stop idlers located on a side of each pulley, such as the side opposite a side having a recess for an articulation wire anchor. One pulley for the up-down articulation and the other for the left-right articulation. Each rotational stop idler may include a separate groove (two total) around the circumference of each sides of the rotational stop idler. The groove terminates at a stop (e.g., a protrusion), preventing the groove from circling three hundred and sixty degrees around the rotational stop idler.

Each pulley includes a boss on the side of the pulley that interfaces with the groove on a first side of a rotational stop idler. During rotation of the pulley relative to the rotational stop idler, the boss on the pulley follows the groove of the idler until it is blocked by the stop on the rotational stop idler. Once the boss on the pulley contacts the stop, the force of the pulley's rotation is transferred to and, may rotate, the idler. Rotation of the idler is permitted depending on the contact of the rotational stop idler and the housing.

On a second side of the rotational stop idler, a second stop interfaces with a boss located on the pulley housing to limit the degree of rotation of the rotational stop idler relative to the housing. For example, during rotation of the idler relative to the housing, the boss on the housing follows the groove on the second side of the rotational stop idler until the second stop member of the idler contacts the boss on the housing, thus stopping the rotation of the idler relative to the housing. Accordingly, after rotation of the pulley relative to the rotational stop idler is prevented in a first direction, and after rotation of the rotational stop idler relative to the housing is prevented in the first direction, rotation of the articulation knob and pulley relative to the housing in the first direction is also prevented.

To prevent the rotation stop idler from wobbling, rotational stop idler may include a continuous bearing surface on both sides of the idler. The continuous bearing surface provides a stable base to keep the idler balanced as it rotates within and against the pulley housing.

Advantageously, the rotational stop idlers have the ability to limit the articulation knob rotation for an endoscope when the articulation knob rotation required exceeds the rotation limits that can be achieved with a single stage stop. On one side of the rotational stop idler, the allowed angular rotation of the pulley relative to idler is equal to the angular length of the groove of the idler minus the angular length of the boss on the pulley. On the other side of the rotational stop idler, the rotation allowed of the idler relative to the housing is equal to the angular length of the groove of the idler minus the angular length of the boss on the pulley housing. These two “stages” and degrees of rotation, when added together, provide the total angular distance the pulley (and/or the articulation knob) may travel relative to the housing. For demonstrative purposes only, if the bosses have an angular length of forty-five degrees each and the grooves of the rotational stop idler each have an angular length of three-hundred fifteen degrees, the degrees of rotation on each side of the idler would equal two hundred and seventy degrees and the sum of the two sides would be five hundred and forty angular degrees of freedom of the pulley relative to the housing. It is understood that the bosses, grooves, and/or stop members may have the same or different angular lengths and that the bosses, grooves, and/or stop members may have angular lengths greater or less than forty-five degrees.

It is also anticipated that one or more of the bosses may be a separate component and that the grooves may follow a spiral path. Accordingly, as the boss follows the path of the groove, the boss may translate along a surface of pulley and/or housing. Advantageously, such arrangements can provide for grooves having an angular length of three hundred sixty degrees or greater.

While the grooves and stops have been described as being defined by the rotational stop idler and the bosses have been described as being of the pulley and/or housing, the arrangements disclosed herein are not so limited. It will be appreciated that the grooves and stops may be defined by the pulley and/or housing and the one or more bosses may be a portion of the rotational stop idler.

Advantageously, the rotational stop idlers limit articulation to reduce the possibility of potential damage to the scope (e.g., the insertion tube) and/or reduce the possibility of injury to the patient. For example, articulation for a cholangioscope may be limited to no more than 60 to 80 degrees so as to reduce the possibility of the insertion tube being articulated to a degree that may cause injury to the bile duct. The rotational stop idler may be configured to a particular type of scope. Accordingly, a first single-use shaft assembly may have rotational stop idlers configured to limit articulation to a first angle, and a second single-use shaft assembly may have rotational stop idlers configured to limit articulation to a second angle that is different from the first angle. The reusable handle assembly; however, may be usable with a variety of types of scopes, including those with rotational stop idlers configured to limit articulation to different angles. Additionally, it is understood that the rotational stop idlers are not limited to multi-piece endoscopes.

The articulation knob and brake assembly of the reusable hand-piece can include an up-down articulation knob, a left-right articulation knob, an up-down articulation brake and a left-right articulation brake. During operation, when the reusable hand-piece is attached to the single-use shaft assembly, articulation of the up-down articulation knob articulates a distal portion of the single-use shaft assembly in a first plane and actuation of the left-right articulation brake knob actuates the distal portion of the single-use shaft assembly in a second plane transverse to the first plane. Preferably, the second plane is orthogonal to the first plane.

When the reusable hand-piece is coupled to the single-use shaft assembly, actuation of the up-down articulation brake lever engages the up-down articulation brake that resists rotation of the up-down articulation knob relative to a portion of the endoscope assembly, such as the housing of the reusable hand-piece. Actuation of the left-right articulation brake knob engages the left-right articulation brake that resists rotation of the left-right articulation knob relative to a portion of endoscope assembly, such as a housing of the single-use shaft assembly.

The reusable hand-piece and the single-use shaft assembly are preferably configured to mate with one another. For example, the reusable hand-piece assembly may define a recess arranged to receive a portion of the single-use shaft assembly, or vice versa. In many examples, the recess of the reusable hand-piece assembly arranged to receive a portion of the single-use shaft assembly will be on a side of the reusable hand-piece assembly opposing the side having the knobs of the articulation knob and brake assembly. One of the reusable hand-piece assembly or the single-use shaft assembly may surround at least a portion of the other, partially and/or completely, on two or more opposing sides.

A portion of the single-use shaft assembly may be receivable in the reusable hand-piece, or vice versa, along a direction parallel-to or transverse to a longitudinal axis of the reusable hand-piece or single-use shaft assembly. For example, the reusable hand-piece may receive the single-use shaft assembly along a direction orthogonal to a longitudinal axis of the reusable hand-piece. In many instances, when joining the reusable hand-piece and single-use shaft assembly to one another, the single-use shaft assembly will move, relative to the reusable hand-piece, along a direction parallel to a rotation axis of the articulation knob and brake assembly.

When the reusable hand-piece assembly is attached to the single-use shaft assembly, the articulation knob and brake assembly cooperates with the single-use shaft assembly to articulate a distal segment of the single-use shaft assembly and/or resist rotation of one or more articulation knobs relative to the endoscope assembly. For example, as discussed in more detail elsewhere herein, one or more articulation shafts and/or brake shafts of the articulation knob and brake assembly may engage the pulley assembly and/or housing of the single-use shaft assembly. One or more shafts of the articulation knob and brake assembly extend through the pulley assembly and engage the housing of the single-use shaft assembly on an opposing side of the pulley assembly from the articulation knob and brake assembly.

In many examples a portion of the articulation knob and brake assembly extends into the recess of the reusable hand-piece arranged to receive a portion of the single-use shaft assembly.

Regarding the up-down articulation control by the articulation knob and brake assembly, the up-down articulation knob is supported on an up-down articulation brake outer housing. Positioned within the up-down articulation brake outer housing is an articulation brake inner housing, an articulation brake position detent, an articulation brake spring, an up-down articulation brake caliper, and an angled articulation brake friction disc. The angled surface of the articulation brake friction disc may be non-perpendicular to the axis of rotation.

Connected to the articulation brake inner housing is the up-down articulation brake lever. Extending through the up-down articulation brake lever is an up-down articulation hub and an up-down articulation shaft. The up-down articulation hub, which is positioned within the articulation brake inner housing, supports the articulation brake position detent and the up-down articulation brake caliper. Rotation of the up-down articulation hub is fixed relative to the housing of the reusable hand-piece which, in turn, fixes rotation of the articulation brake position detent and the up-down articulation brake caliper relative to the housing of the reusable hand-piece as well.

Upon rotation of the up-down articulation brake lever relative to the up-down articulation hub, the articulation brake inner housing rotates relative to the articulation brake position detent and the up-down articulation brake caliper. Upon rotation in a first direction, pins extending from the up-down articulation brake inner housing towards the up-down articulation brake caliper engage angled surfaces of the up-down articulation brake caliper to axially move the articulation brake caliper towards the articulation brake spring to compress the articulation brake spring. Upon rotation in a second direction, the pins move along the angled surfaces to allow the articulation brake spring to force the up-down articulation brake caliper towards the angled surface of the articulation brake friction disc and the up-down articulation brake outer housing to compresses the articulation brake friction disc between the up-down articulation brake caliper and the up-down articulation brake outer housing. This contact between the stationary (relative to the reusable hand-piece) up-down articulation brake caliper, the angled surface of the articulation brake friction disc, and the up-down articulation brake outer housing results in resistance of the up-down articulation knob, which is supported by the up-down articulation brake outer housing, rotating relative to the reusable hand-piece. In other examples, the articulation brake surface may be perpendicular to the axis of rotation. Advantageously, angled brake surfaces can increase the braking force in comparison to the perpendicular brake surface at equivalent applied spring force.

As rotation of the up-down articulation brake position detent is fixed relative to the housing of the reusable hand-piece via the up-down articulation brake hub, rotation of the articulation brake inner housing relative to the housing also rotates the articulation brake inner housing relative to the up-down articulation brake position detent. The up-down articulation brake position detent may be arranged to retain the up-down articulation brake inner housing in one or more positions. For example, the detent may retain the up-down articulation brake inner housing in a first position in which braking resistance is applied via up-down articulation brake friction disc interaction between the up-down articulation brake caliper and the up-down articulation brake outer housing and/or in a second position one in which braking resistance is not applied. The brake inner housing and/or detent may be arranged for retaining the up-down articulation brake inner housing in any of a plurality of positions to provide an adjustable level of rotational resistance to the articulation control (e.g., articulation control knobs).

Extending through the center of the up-down articulation brake hub is an up-down articulation shaft. The up-down articulation shaft has a first end rotationally fixed relative to the up-down articulation brake outer housing. A second end of the up-down articulation shaft communicates with the pulley assembly of the single-use shaft assembly. For example, the second end of the up-down articulation shaft may be attached to the up-down articulation pulley such that rotation of the up-down articulation shaft rotates the up-down articulation pulley. In this way, rotation of the up-down articulation knob rotates the up-down articulation brake outer housing which rotates the up-down articulation shaft which rotates the up-down articulation pulley which actuates the up-down articulation wire(s).

Regarding left-right articulation, the structure and operation of the left-right articulation knob and brake assembly is the same as the up-down articulation knob and brake assembly with the exception that rotation of the left-right articulation brake hub is fixed relative to the housing of the single-use shaft assembly and that such rotation is fixed via a left-right articulation brake shaft extending through the left-right articulation shaft. Similar to the up-down articulation knob and brake assembly, the left-right articulation knob is supported by a left-right articulation brake outer housing which contains an angled articulation brake friction disc, a left-right articulation brake caliper, an articulation brake spring, an articulation brake position detent and an articulation brake inner housing.

Extending through the left-right articulation brake knob and the left right articulation brake outer housing is the left-right articulation shaft. The left-right articulation shaft has a first end rotationally fixed relative to the left-right articulation brake outer housing and a second end engaging the pulley assembly of the single-use shaft assembly (e.g., the second end rotationally fixed to the left-right articulation pulley) such that rotation of the left-right articulation brake knob rotates the left-right articulation brake outer housing which rotates the left-right articulation brake shaft which in turn rotates the left-right articulation pulley which in turn translates the left-right articulation wire(s).

The left-right articulation brake shaft, which extends through the left-right articulation shaft, has a first end rotationally fixed to left-right articulation brake hub and a second end rotationally fixed relative to the housing of the single-use shaft assembly when the reusable hand-piece assembly is attached to the single-use shaft assembly. Similar to the operation of the up-down articulation knob and brake assembly, rotation of the articulation brake inner housing relative to the left-right articulation brake caliper moves the left-right articulation brake caliper towards or away from the left-right articulation brake outer housing and the angled surface of the brake friction disc positioned therebetween. The left-right articulation brake knob is rotationally fixed to the left-right articulation brake inner housing such that rotation of the left-right articulation brake knob rotates the left-right articulation brake inner housing relative to the left-right articulation brake hub, the left-right articulation brake caliper, and the left-right articulation brake position detent. Accordingly upon engagement of the left-right articulation brake, the left-right articulation brake caliper and angled surface of the brake friction disc resist rotation of the left-right articulation brake outer housing and left-right articulation knob relative to the left-right articulation brake shaft and the single-use shaft assembly.

The second end of the left-right articulation brake shaft may be arranged to slidably engage with the housing of the single-use shaft assembly and rotationally fix the left-right articulation brake shaft relative thereto. Positioned circumferentially around the left-right articulation brake shaft is the left-right articulation shaft with its second end arranged to engage the left-right articulation pulley cooperating with the left-right articulation wire(s). And, positioned circumferentially around the left-right articulation shaft is the up-down articulation shaft having its second end arranged to engage the up-down articulation pulley associated with the up-down articulation wire(s).

The articulation knob and brake assembly may include a plurality of O-rings between the various components when assembled. Also illustrated are ball bearings disposed within the assembly to reduce the friction created during rotation.

The up-down articulation brake lever can have a surface configured to mate with a surface of the up-down articulation inner housing such that rotation of the up-down articulation brake lever rotates the up-down articulation brake inner housing relative to the housing of the reusable hand-piece assembly. Preferably, the geometry of surface interferes with the geometry of surface during rotation. The up-down articulation brake inner housing can define one or more apertures arranged to receive transverse pins that engage the angled surfaces of the up-down articulation brake caliper. Additionally, an inner surface of the up-down articulation brake inner housing can include recesses or protrusions arranged to engage detent spring. The up-down articulation brake lever and the up-down articulation brake inner housing define an aperture for receiving the up-down articulation hub.

The up-down articulation hub has a portion that extends through the up-down articulation brake position detent and the up-down articulation brake caliper. Preferably portion, the up-down articulation brake position detent, and/or the up-down articulation brake caliper have a cross-sectional geometry that fixes the up-down articulation brake detent, the up-down articulation brake caliper, and/or the up-down articulation brake hub from rotation relative to one another.

The up-down articulation brake outer housing may be engaged with and rotationally fixed relative to the up-down articulation shaft. The up-down articulation brake outer housing has a portion arranged to rotationally fix the up-down articulation knob thereto (e.g., via recesses arranged to receive protrusions of the up-down articulation knob or vice versa).

The inside of the up-down articulation brake outer housing may have an inner friction surface arranged to engage the angled/tapered surface of the up-down articulation brake friction disc when the up-down articulation brake is engaged.

When the up-down articulation brake lever is rotated relative to the housing of the reusable hand-piece, the up-down articulation brake inner housing rotates with the up-down articulation brake lever and moves pins extending from the apertures of the up-down articulation brake inner housing relative to the up-down articulation brake caliper. The pins move along the sloped surfaces of the up-down articulation brake caliper to translate the up-down articulation brake caliper along the up-down articulation hub in cooperation with the up-down articulation brake spring. When configuring the up-down articulation brake from a disengaged configuration to an engaged configuration, the up-down articulation brake caliper forces the up-down articulation brake friction disc against the up-down articulation brake outer housing (compressing the up-down articulation brake friction disc). When configuring the up-down articulation brake from an engaged configuration to a disengaged configuration, the up-down articulation brake caliper moves away from the up-down articulation brake housing and the up-down articulation brake friction disc and compresses (e.g., further compresses) the up-down articulation brake spring. The up-down articulation brake may be in the disengaged configuration with a gap between the up-down articulation brake friction disc and the inner friction surface of the up-down articulation brake outer housing.

The up-down articulation brake inner housing may have detent recesses or protrusions to engage one or more protrusions or recesses of the up-down articulation brake position detent. Such an arrangement may provide securement of the up-down articulation brake lever in one or more positions and/or provide a tactile and/or audible indication of the up-down articulation brake entering and/or leaving the one or more configurations or positions.

The left-right articulation knob and brake assembly work in a similar fashion to the up-down articulation knob and brake assembly with the exception of the left-right articulation brake hub being rotatably secured relative to the housing of the endoscope assembly by a left-right articulation brake shaft extending centrally through the left-right articulation shaft and the up-down articulation shaft.

The left-right articulation brake outer housing and the left-right articulation shaft are disclosed assembled and rotationally fixed relative to one another.

The left-right articulation brake shaft has a first end received within and rotationally fixed to the left-right articulation brake hub. A second end of the left-right articulation brake shaft is arranged to engage the housing of the single-use shaft assembly during attachment of the reusable hand-piece assembly and the single-use shaft assembly and rotationally fixe the left-right articulation brake shaft relative to the single-use shaft assembly. Accordingly, when the reusable hand-piece assembly is attached to the single-use shaft assembly, the left-right articulation brake hub is rotationally fixed relative to the housing of the single-use shaft assembly.

The up-down articulation knob, the left-right articulation knob and the left-right articulation brake knob may be detachable (or “snap off”) from the rest of the articulation knob and brake assembly. As discussed above, the up-down articulation brake outer housing has a portion arranged to rotationally fix the up-down articulation knob thereto (e.g., via recesses arranged to receive protrusions of the up-down articulation knob or vice versa). Similarly, the left-right articulation brake outer housing is arranged to fix the left-right articulation knob in the same fashion. The recesses and protrusions arrangement allow the up-down articulation knob, left-right articulation knob and left-right articulation brake knob to be easily snapped off or snapped on to the articulation knob and brake assembly. Advantageously, the snap off feature allows the knobs to be removed to facilitate cleaning of the reusable hand-piece between procedures.

The reusable hand-piece may include a retention clip. Retention clip is used to secure articulation knob and brake assembly to the reusable hand-piece. The retention clip may be coupled with the up-down articulation hub to secure the articulation knob and brake assembly. In between procedures, to facilitate quick and easy cleaning, the retention clip may be removed from the articulation and brake assembly, separating the articulation and brake assembly from the housing of the reusable hand-piece.

The insertion tube may include an instrument tube extending within the interior of at least a portion of the insertion tube. The instrument tube defines a tool channel allowing various instruments to be inserted and extended through the insertion tube. The instrument tube may be created through a coextrusion process. The inner extrusion may be comprised of High Density Poly Ethylene (HDPE) for lubricity. The outer extrusion may be formed of polyvinyl chloride (PVC) or polylactic acid (PLA) to facilitate bonding.

The insertion tube may include an optical sensor module for providing readable information by the console and/or project an image on a monitor such as a video feed from the end of the insertion tube. This video feed can be infrared, thermal or visible light. The insertion tube may include a light-emitting diode (LED) wire for providing power to an LED at the end of the insertion tube.

The insertion tube may include fluid paths discussed herein defined by tubes, such as a suction tube, tissue irrigation tube, camera flush tube, and/or insufflation tube. The irrigation tube allows the user to clear the visual field of any debris during use of the instrument. Often, this involves the removal of excess blood so the place of interest may be visualized. The suction tubing allows for the aspiration of particulate matter or various secretions from inside the patient's body. The camera flush tube and insufflation tube may be combined by a Y fitting. Various combinations of the tubing are envisioned by the applicant to minimize the number of tubes required in the insertion tube.

Further disclosed is an example of an articulating joint assembly located in a flexible articulation section of the insertion tube. The articulating joint assembly may comprise a proximal end and a distal end. Configured between the proximal end and the distal end are a plurality of connected articulation links. Each link has a contact point where the link is connected to the adjacent link. Adjacent links are arranged to articulate relative to one another. For example, adjacent articulation links may be arranged to pivot an angle relative to one another. For example, adjacent links may be arranged to pivot an angle of 2x relative to one another, where angle (x) is measured between contact points of each adjacent link when the links are parallel to one another. Adjacent articulation links in the articulating joint assembly may be pivotable to different degrees than other adjacent articulation links in the articulating joint assembly. Preferably, adjacent articulating links may be formed so that the angles of articulation are different between different pairs of the articulation links. Advantageously, such an arrangement may be used to obtain a variable radius along the length of the flexible articulation section during articulation. Pairs of adjacent links having different angles of articulation may be used to create a variable curve along the length of the flexible articulation section.

Further disclosed is an elevator control assembly for adjusting the height of an elevator at a distal end of the insertion tube. The elevator control assembly may be disposed between the two inner pieces of the pulley housing. The elevator control assembly includes an elevator control lever. The elevator control lever articulates around the articulation shafts via pins constrained by curvilinear tracks. The elevator control lever being operationally coupled to an elevator driving rack. The elevator driving rack is coupled to an elevator pinion. The elevator pinion is also operationally coupled to an elevator driven rack. The elevator driven rack is coupled to an elevator articulation control wire. In addition, the elevator control assembly may include an elevator control coil termination block that receives one or more elevator control coils wrapping around respective elevator articulation control wires connected to the elevator driven rack.

Articulation of the elevator control lever operates the elevator driving rack, which in turn rotates the elevator pinion. When the elevator pinion is rotated, it drives the elevator driven rack to articulate an elevator articulation control wire with elector control coils wrapped around the wire. For example, actuating the elevator control lever in one direction causes the elevator height to increase, while articulation in the opposing direction causes the elevator height to decrease.

In order to maintain the position of the elevator control lever when user input is not provided, the elevator control assembly utilizes a compressed o-ring. The o-ring is secured around a protrusion extending from one of the housing covers. The o-ring is pressed against the elevator control pinion, maintaining the position of the control lever utilizing the friction created between the o-ring and the elevator control pinion. In other examples, the position of the lever is maintained utilizing friction created by a spring element.

Further disclosed is an insertion tube tip assembly. The insertion tube tip assembly includes the insertion tube, a distal elevator link, a distal cap and an elevator assembly. The insertion tube includes distal end. The distal end includes a distal tip including the distal elevator link, distal cap and the elevator assembly.

The distal cap includes a first opening to slide over the elevator assembly. The distal cap further includes a second opening to allow instruments to exit through the distal cap and operate within the patient's body. The distal cap further includes a notch on a top portion of the distal cap. The distal cap may be removable from the elevator assembly and/or distal portion of the insertion tube. In some examples, the distal cap is not included.

The elevator assembly includes an opening to extend instruments out of the instrument tube. The instrument tube may extend through a portion of the insertion tube. The opening opens into a working chamber. The chamber may be defined by opposing side walls, a distal surface of the insertion tube and an elevator. Adjacent to the insertion tube opening is the elevator. The elevator may be used to adjust the elevation of an instrument within the patient's body to access the desired location within the body during a procedure. The elevator may include a proximal end and a distal end. The elevator may be secured to the elevator assembly by a pivot pin. During articulation of the elevator, the elevator rotates around the pivot pin, allowing the distal end of the elevator to be deflected up or down. The elevator may include a notch at the distal end of the elevator opposing the notch of the distal cap. The opposing notches may be utilized to limit unintended longitudinal or lateral movements of instruments passing through the instrument channel and into the patient's body. Notch may hold an instrument protruding out of opening to increase the control of the instrument.

The elevator assembly further includes an elevator control wire. Elevator control wire may be a single strand pre-formed wire for articulation of the elevator. The elevator wire may translate over a static curved ramp resulting in a rotational torque on the elevator. Elevator wire is manipulated by the elevator control assembly to control the height of the elevator.

The elevator assembly may further define a light window through which a light may be admitted, such as from a light emitting diode. This arrangement allows the user to illuminate a patient's body during a procedure. The elevator assembly may further define an imaging window to provide images back to the endoscopist.

The present disclosure further provides a system comprising an endoscope assembly, a cartridge and a console. The system may include the cartridge inserted into a control valve assembly of the console. The cartridge can be connected to the endoscope by the umbilical. For instance, the cartridge may be connected to a single-use shaft assembly of the endoscope by one or more fluid tubes and/or electrical conductors.

The cartridge may be connected to support equipment. The support equipment may include an irrigation pump, an insufflator, and/or a vacuum pump (e.g., stand-alone or central). The cartridge may be positioned between the endoscope and the support equipment.

In some examples, the console may include a user interface allowing the user to alter the various variables of the endoscope system. In one example, the user interface may be a touchscreen integrated into an electric visual display. In another example, the user interface could include a keyboard, mouse, trackball, and/or touch sensitive pointing device, etc.

The console may include a control valve assembly for receiving and actuating upon a cartridge. The console may provide electrical power, fluids and/or fluid pressure, vacuum pressure, and/or send and/or receive electrical signals with the endoscope assembly. The console may include a monitor for visualizing signals received from the endoscope assembly such as a video feed from a camera at the distal end of the single-use shaft assembly.

The control valve assembly may be used to selectively actuate an actuatable portion of fluid paths of the cartridge. The control valve assembly may include actuators and a support structure comprising a first surface and a second surface.

The actuator may be a linear actuator or rotational actuator. The actuator may be a solenoid. Although as few as one actuator is appreciated by the applicant, in preferred examples the control valve assembly includes four actuators. The actuators are arranged to actuate valve portions of fluid paths in the cartridge to selectively open and/or close the fluid paths.

The valve portions of the fluid paths may comprise any acceptable valve. For example, the valve portion may comprise a petcock. The valve portion may also comprise a portion of tubing arranged for compression (e.g., a pinch valve) between the anvil on the first surface of the door and the actuator (e.g., solenoid) of the control valve assembly. The valve portions may serve as a valve for any number of fluid functions of the endoscope, such as a suction valve associated with actuator, a camera flush valve associated with actuator, an insufflation valve associated with actuator, and/or an insufflation vent valve associated with actuator.

The actuators of the control valve assembly include electrical connectors to be received by the console and electrically connect the actuators to the console. When connected to the console, the console can provide electrical power and user inputted commands to the actuator to open and/or close the fluid pathways of the cartridge.

The cartridge may include an identifier readable and/or writable by the control valve assembly. The identifier may include information regarding the type of cartridge and/or usage information (e.g., whether the cartridge has been used before). Preferably, the control valve assembly is configured to read and process the identifier of the cartridge (e.g., to determine the type of cartridge and/or whether the cartridge has been used previously). The identifier may be readable and/or writable through an/the electrical connector of the cartridge and/or through other means (e.g., RFID, optics, and/or mechanical contact).

The first surface of the support structure may include the electrical connector for forming electrical connections to the electrical connector of the cartridge. The electrical connection between the control valve assembly of the console and the cartridge may allow the console to receive images and control data (valve/actuator and/or image data) passed from the endoscope through the cartridge to the console. The electrical connection may allow the console to supply power to the light-emitting diode (LED) of the endoscope assembly.

The cartridge can be a single-use cartridge. The single-use cartridge can negate the need for the use of permanent valve bodies contained within a reusable endoscope and/or the console. This advantageously eliminates the need to sterilize reusable valves after every use.

The cartridge has a housing. The housing can include a first side and a second side. The housing further comprises a first fluid side and a second fluid side allowing for fluid to travel in and out of the cartridge housing. The housing may define one or more fluid pathways, fluid connector, and/or electrical connectors. For example, cartridge housing can include electrical connector in electrical communication with an electrical conductor of the endoscope and optionally an optical sensor module that contains optical sensor calibration data or a unique identifier for the endoscope. The housing may define windows. In one example, the housing includes four windows. The windows may be defined by inner surfaces of the housing. The inner surface may create a generally circular opening in the housing. The circumference of one window may be larger than the other windows to receive a larger fluid path cross section.

The windows can be configured to receive an actuator from the console into a chamber defined by the inner surface. As described above, the actuator may pass through the window and/or contact an anvil on the door of the control valve assembly of the console to create a pinch valve with the door. When the cartridge is positioned on the control valve assembly and the door is closed, the windows align with the anvils on the door and the actuators of the control valve assembly. The windows may be arranged to define a geometric shape. For instance, as illustrated, the windows may define a diamond.

As described above, one or more actuators may be solenoids configured to extend into the windows of the cartridge housing and/or contact the anvils on the door of the control valve assembly creating a pinch valve when closed.

The housing may further include an electrical connector for connecting to the electrical connector of the control valve assembly. The electrical connector of the cartridge may be spring finger electrical contacts configured to contact a corresponding number of flat pad electrical contacts on the control valve assembly of the console or vice versa. The electrical connector of the cartridge housing may pass images and control data provided by the endoscope to the console. The electrical connector of the housing may pass power from the console to the LED used by the endoscope in the patient's body.

The cartridge may be inserted into the control valve assembly of the console. The cartridge is inserted between the first surface of the support structure of the control valve assembly. The first fluid side of the cartridge may include loose tubing that continues into the umbilical and to the single-use shaft assembly of the endoscope.

The second fluid side of the cartridge may be connected to support equipment. The support equipment may include an irrigation pump, an insufflator, and/or a vacuum pump (stand-alone or central). The cartridge may provide for fluid communication between the endoscope and the support equipment.

The control valve assembly may include a ledge surface and a latch. The cartridge may include a ledge surface and a latch receiving portion. In alternate examples, the control valve assembly may include the latch receiving portion and the cartridge may include the latch. When the cartridge is received by the control valve assembly the latch contacts the latch receiving portion to secure a first end of the cartridge to the control valve assembly. Separately, and opposite of the latch and latch receiving portion, the ledge surface of the cartridge and the ledge surface of the control valve assembly contact each other to secure a second end of the cartridge to the control valve assembly.

In some examples, the cartridge may include two or more magnets located on the first side of the cartridge. In some examples, one magnet may be located at the bottom of the first side of the cartridge and the second magnet located at the top of the first side of the cartridge. The first surface of the control valve assembly may include two or more magnetic sensors (hall or reed), mirroring the locating of the magnets on the cartridge. For example, one located at the bottom of the first surface and the other located at the top of the first surface. The bottom magnet on the cartridge interfaces with the bottom magnetic sensor on the control valve assembly triggering the actuator (e.g., solenoid) valves to retract reducing the force required to tilt the cartridge into the control valve assembly. The top magnet interfaces with the top magnetic sensor on the control valve assembly to confirm the presence of the cartridge on the console, triggering the console to release the appropriate actuators and initiate the actuator operation mode for endoscopy.

The console may be coupled with a first cartridge, a second cartridge. The first cartridge may be connected to a first endoscope and the second cartridge may be connected to a second endoscope. The first cartridge and the second cartridge may be securely inserted into a first control valve assembly and a second control valve assembly of the console, respectively. Although the console is illustrated with the first and second control valve assembly, the console may have any number control valve assemblies each capable of receiving an independent cartridge. The first control valve assembly may receive the first cartridge and the second control valve assembly may receive the second cartridge. It is not necessary for every control valve assembly to receive a cartridge during every medical procedure.

As illustrated, each cartridge may be connected to a separate set of support equipment and an independent medical device, such as an endoscope.

The first control valve assembly and the second control valve assembly are configured to operate independently and/or in a main/secondary communication configuration. The first or mother endoscope may be the main controller and the second or daughter endoscope may be the secondary controller. In this arrangement, the controls of the main endoscope may control the fluid functions (e.g., image controls and/or LED) of one or more secondary endoscopes. The main endoscopes may control the function of the fluid paths of the secondary endoscopes by providing an electrical signal to the second control valve assembly to selectively actuate an actuator as described herein (e.g., through one or more cartridges connected to the console). The first endoscope may further control a function of the second endoscope (e.g., operation of the LED and/or camera) by providing an electrical signal from the first endoscope to alter a condition of the second control valve assembly (e.g., alter an electrical signal provided by the second control valve assembly to the cartridge of the second endoscope). For instance, the control valve assembly may provide an electrical signal to the LED and/or the camera of the second endoscope based on the signal received from the first endoscope. This arrangement further allows for multiple endoscopes to be setup and utilized in a procedure at once, eliminating or reducing extensive setup time typically used to change one endoscope out for another endoscope and/or eliminating or reducing the need for additional equipment, such as multiple control consoles.

The console is illustrated with a touchscreen user interface. The user interface may be any suitable input/output device.

The single-use shaft assembly can include an insertion tube, an umbilical connected to the cartridge and therefore the console, an access port for selectively accessing a lumen of the single-use shaft assembly (e.g., for taking a sample such as a biopsy) and/or for delivering a fluid through a lumen of the single-use shaft assembly, and/or an electrical connector for electrically coupling the single-use shaft assembly to the reusable hand-piece.

The umbilical can extend from the cartridge to the single-use shaft assembly of the endoscope. Extending within the umbilical to the distal tip of the insertion tube are several fluid paths and/or electrical wires. The fluid paths may include paths for suction, tissue irrigation, camera flush, and/or insufflation. The fluid pathways can be free of valves from the distal tip of the endoscope to the end of the umbilical. The fluid pathways may include valves only within the boundary of the cartridge.

Preferably, one or more fluid paths are defined by monolithic tubing extending from the single use shaft assembly to the cartridge. More preferably, the monolithic tubing extends through the cartridge and out of the second fluid side of the cartridge.

The camera flush fluid pathway and insufflation fluid pathway may be combined by a T-fitting in the single-use shaft assembly into a combined camera flush and insufflation fluid pathway. This allows the pressure from insufflation to be used to push water from a water source through the camera flush fluid pathway and out the distal end to clean the camera lens of debris collected during a procedure.

The umbilical may further include an image data conductor, control data conductor, and/or an LED power conductor. The image data conductor and/or LED power conductor may extend through the single-use shaft assembly to the distal tip of the insertion tube. The image data conductor may allow the transmission of the image data taken from the camera back to the console. The LED power conductor may provide power from the console to the LED at the distal tip of the insertion tube, thus providing illumination for the procedure. The control data conductor allows the communication of control data from the controls of the reusable hand piece to the cartridge which, in turn, allows communication to the console.

Further disclosed is a packaging system for and method for maintaining sterility of a medical product, such as an endoscope assembly or a portion of an endoscope assembly. Although the disclosed packaging system can be used for a number of medical products, the disclosure will refer to the medical product as an endoscope assembly. In one example, the packaging assembly includes an elongated tray with a first end and a second end. The tray defines a recess for retaining the endoscope assembly. In some examples, the tray may be manufactured by thermoforming and may be made of a recyclable material that allows the tray to be recycled after use.

The tray may be formed such that the medical product may only be placed within the tray in one configuration. The tray may be thermoformed. The tray preferably defines a recess. The recess preferably defines multiple compartments to house the various components of the endoscope assembly. The recess may include an insertion tube compartment, single-use shaft assembly compartment, an umbilical compartment and a cartridge compartment. The recess may include additional compartments if additional components are stored within the tray. The recess may include fewer compartments if fewer components are stored within the same tray. For instance, the cartridge may be sold separately or in a separate container within the packaging system, therefore, there would not be a need for a cartridge compartment within the tray.

The tray may include hanger slots at the first end of the tray, for example a pair of hanger slots. The hanger slots may be attached as a separate piece or formed out of the material forming the tray. The hanger slots allow the elongated package to be hung from a support arm. The support arm may be attached to the wall or ceiling of the operating room. In some examples, the support arm may be attached to a cart. When attached to the support arm in a vertical orientation a portion or the entirety of the tray is suspended above the floor.

The packaging system may further include a cover (e.g., a sterile barrier) extending over and sealing the recess portion of the tray from outside contaminants and retaining the endoscope assembly in a sterilely sealed condition. The cover may be a peel-away cover allowing a user to easily access the endoscope assembly. The cover may be made of a high-density polyethylene fibers, such as Tyvek®.

In some examples, the tray may contain a two piece endoscope including a reusable hand piece and single-use shaft assembly. In the illustrated examples, only the single-use shaft assembly is included in the packaging system. The single-use shaft assembly may include an insertion tube at the patient end of the endoscope. The single-use shaft assembly may further include an umbilical extending to and connecting to a cartridge. Any combination of the components within the tray may be provided connected and/or fully assembled with the other components.

The packaging system may include a first cover, in some examples the packaging system may contain more than one cover. In the illustrated example, a first cover is used to retain the cartridge within the tray (e.g., the cartridge compartment). The packaging system may include a second cover to retain the single-use shaft assembly within the tray (e.g., the single-use shaft assembly compartment).

During preparation for a procedure, a user may peel away the cover allowing access to the endoscope assembly. As used herein, user may mean an endoscopist, a nurse, a technician, a doctor or any other individual who may be present prior to or during a procedure. In some examples, during set up, the cover may be peeled away a portion at a time. By only peeling the cover (e.g., sterile barrier) partially, the user may set up the endoscope for procedure while keeping the rest of the endoscope assembly sterile and/or reducing the risk of contamination. The first cover may be pulled away to reveal the cartridge. The cartridge and umbilical may be removed in order to attach the cartridge to the console.

Advantageously, by peeling away only a portion of the cover (e.g., sterile barrier) and/or fewer than all of the covers, the handle and insertion tube are still protected from various contaminants. Often, the package system is hung from a support arm. When the package is open the insertion tube may swing during set up/preparation causing the insertion tube to bang against the cart, cart wheels or individuals as they maneuver throughout the room. By keeping the insertion tube covered by the cover (e.g., sterile barrier), although the sterile seal may have been broken, the cover is still protecting the insertion tube from picking up various contaminants by contact anything within the environment.

Next, the second cover may be pulled back to access the single-use shaft assembly in the single-use shaft assembly compartment. The second cover (e.g., sterile barrier) may be peeled away further in order to complete this step, depending on whether the user is peeling the cover away a portion at a time or all at once. In some examples, the first cover must be pulled away before the second cover can be pulled away, requiring the user to follow the same set up procedure. In other examples, the second cover is independent of the first cover, allowing both covers to be accessed separately. Once the second cover has been pulled away, the single-use shaft assembly may be removed for set up. To facilitate easy removal of the single-use shaft assembly and the insertion tube, the covers may be configured to slide vertically along the sides of the tray in order to expose more of the insertion tube.

The reusable hand-piece may be held by a support arm. Once the single-use shaft assembly has been removed from the packaging system, the user may couple the single-use shaft assembly to the reusable hand-piece.

A second example may include a curved flexible endoscope packaging. The packaging assembly includes tray. The tray defines a recess for retaining the endoscope assembly. In some examples, the tray may be manufactured by thermoforming and may be made of a recyclable material that allows the tray to be recycled after use.

The tray may be formed such that the medical product may only be placed within the tray in one configuration. The tray may be thermoformed. The tray preferably defines a recess. The recess preferably defines multiple compartments/tracks to house the various components of the endoscope assembly. The recess may include an insertion tube track, single-use shaft assembly compartment, an umbilical track and a cartridge compartment. The curved packaging system may further include a suction tube compartment to retain the suction tube. Unlike the elongated tray, the compartments for the insertion tube and umbilical encircle the center of the tray. The umbilical and insertion tube are wound around the center of the packaging. The umbilical and insertion tube tracks may overlap. When the tracks overlap, the umbilical may be positioned on top of the insertion tube. The recess may include additional compartments if additional components are stored within the tray. The recess may include fewer compartments if fewer components are stored within the same tray. For instance, the cartridge may be sold separately or in a separate container within the packaging system, therefore, there would not be a need for a cartridge compartment within the tray.

The tray may include hanger slots at the top end of the tray, for example a pair of hanger slots. The hanger slots may be attached as a separate piece and/or defined by the material forming the tray. The hanger slots allow the elongated package to be hung from a support arm. The support arm may be attached to the wall or ceiling of the operating room. In some examples, the support arm may be attached to a cart. When attached to the support arm the entirety of the tray is suspended above the floor. Due to the compact configuration of the curved packaging system, the tray may be placed on a cart rather than hung from a support arm if desired.

The packaging system may further include a cover (e.g., sterile barrier) extending over and/or sealing the recess portion of the tray from outside contaminants and retaining the endoscope assembly in a sterilely sealed condition. The cover may be a peel-away cover allowing a user to easily access the endoscope assembly. The cover may be made of a high-density polyethylene fibers, such as Tyvek®.

Similar to the elongated packaging system, the curved packaging system may include a first cover, in some examples the packaging system may contain more than one cover. In the illustrated example, the first cover is used to retain the cartridge within the tray/cartridge compartment. The packaging system may include a second cover to retain the single-use shaft assembly within the tray/single-use shaft assembly compartment.

Similar to the elongated packaging system, the curved packaging system may be designed to encourage the user to remove the endoscope assembly in a desired sequence. During set up, the cover (e.g., sterile barrier) may be partially and/or completely removed (e.g., peeled away) to access the entirety of the endoscope assembly. Due to the configuration of the packaging system, the dangers of contamination present in the elongated packaging system are reduced.

First the first cover retaining the cartridge may be moved to access the cartridge. The cartridge and umbilical may be removed and the cartridge may be attached to the console. Next, the second cover may be moved to access the single-use shaft assembly. The single-use shaft assembly may be removed from the tray and attached to the reusable hand piece assembly. Finally, the insertion tube may be removed and inserted into the patient or placed on a sterile surface until the procedure commences.

Advantageously, the packaging system allows an individual or multiple individuals to unpack the medical device in sequence, in preparation for a procedure protecting the components still in the packaging from contamination from the surrounding environment during setup.

Further disclosed is a cartridge with a suction tube extension incorporating a t-fitting. This configuration may be used with a flexible single-use viewing endoscope that incorporates a suction tube with an inline t-fitting. The t-fitting may include a removable cap. The removable cap protecting a side port of the t-fitting from admitting contaminants when the t-fitting is not in use and removable when use is desired. The side port of the t-fitting may mate with a connector on a cholangioscope suction tube.

Further disclosed is a method of selecting between the imaging devices of one or more endoscopes to display on a monitor during an endoscopic procedure. Often, during endoscopic procedures, multiple endoscopes are utilized to perform the procedure. This method is directed toward identifying which endoscope's image to display on the monitor. In some embodiments, multiple monitors may be used. In other embodiments, a single monitor may be used to display all of the endoscopes' images on one monitor. When using one monitor, the endoscope being used may be identified and displayed as the most dominate or largest image being displayed among other smaller images on the same screen/monitor.

The method may include identifying the endoscope being used at a certain point during a procedure. The endoscope may be identified using a switch, for example a mechanical, inductive or capacitive switch (e.g., a touch switch). The switch may be a sensor. In some embodiments, the switch is a touch switch, so that when an endoscope is picked up by an endoscopist, the switch is activated so as to identify the endoscope currently being used in the procedure. When the switch is activated, a signal is relayed from the switch to a controller, such as to identify which endoscope is being used. The controller relays the signal/image from the associated endoscope to a monitor. The identified image being the dominate image on the screen while the endoscope is still in use. In some examples, multiple endoscopes may be used simultaneously. When multiple endoscopes are used simultaneously, the images from the endoscopes in use may be displayed on the monitor as the same size. In other examples, a dominate endoscope may be identified. When the dominate endoscope is being used simultaneously with another endoscope, the image relayed from the dominate endoscope may be the largest image on the monitor.

Further disclosed are three embodiments of image sensor assemblies used in various types of endoscopes, for example gastroscopes, colonoscopes, duodenoscopes or cholangioscopes. Broadly, the camera and LED board may be arranged at the distal end of an insertion tube and a cable with electrical connectors may extend from the distal end of the insertion tube to the proximal end of the insertion tube and to the electrical connector/circuit board in the cartridge. As described above, the electrical connector of the cartridge interfaces with the console and passes electrical signals between the cartridge and the console. In some embodiments, the electrical connector of the cartridge may include spring finger electrical connectors to contact with spring fingers on the console.

The image sensor assembly includes a camera and LED board. A side of the camera and LED board is bonded with a cable. The cable may be bonded to the board with any method know in the art, for example with an adhesive. Extending from the distal end of the cable and soldered to the board are a plurality of wires (not shown). The wires are soldered to the board to carry signals between the cartridge and the camera and LED. The other ends of the same wires extend from the proximal portion of the cable and are soldered on the surface of a flex circuit. The wires may be appropriately spaced apart across the surface of the flex circuit.

The flex circuit may be operably attached to a micro connector. The micro connector may be either the male or female portion with the opposite portion located on the electrical connector of the cartridge. The male and female portions are connected to complete the electrical path between the camera and/or LED and the cartridge. In some embodiments, the micro connector may be a mezzanine connector.

During assembly, the proximal end of the wire including the flex circuit and micro connector are passed into the distal end of the insertion tube and through the length of the insertion tube towards the proximal end of the insertion tube. The flex circuit and micro connector may then be passed through the endoscope and umbilical to the cartridge operably connected at the end of the umbilical. The female or male portion of the micro connector located on the flex circuit is then connected with the corresponding portion of the micro connector located on the electrical connector of the cartridge. Advantageously, this method of manufacturing avoids passing the cable through the insertion tube before connecting the camera and LED board to the cable, which can be challenging and time consuming. Moreover, the method avoids passing the camera through the insertion tube, which may damage the camera and/or require a larger insertion tube to accommodate the camera.

The flex circuit and micro connector preferably have a combined height of less than 1 mm. More preferably, the combined height of the flex circuit and micro connector is less than 0.8 mm. Such arrangements may have a combined height in the range of 0.6 mm to 0.8 mm. The flex circuit itself may have a height of about 0.2 mm.

The circuit board may further include a storage device to store the imaging data from the camera.

The image sensor assembly may further include a second micro connector on the cartridge electrical connector. The second micro connector is also attached to a flex circuit similarly to the arrangement in the description above. The flex circuit includes a wire extending from the flex circuit on the cartridge circuit board to a single-use shaft assembly electrical connector. The single-use shaft assembly electrical connector is connectable to the electric connector of the reusable hand-piece to pass electrical signals/commands between the two endoscope pieces.

Any of the inventive aspects and examples discussed herein may be used independently or in combination with each other. A single medical device may include any and/or all the aspects disclosed herein.

Other aspects, objectives, and advantages of the present disclosure will become more apparent from the following detailed description when taken in conjunction with the accompanying drawings. Further forms, objects, features, aspects, benefits, advantages, and examples of the present disclosure will become apparent from a detailed description and drawings provided herewith.

### BRIEF DESCRIPTION OF THE DRAWINGS

FIG. 1 illustrates an endoscope assembly, cartridge and console.

FIGS. 2 and 3 illustrate the endoscope assembly of FIG. 1.

FIG. 4 illustrates an exploded view of the endoscope assembly of FIG. 1.

FIG. 5 illustrates a single-use shaft assembly of an endoscope assembly.

FIG. 6 illustrates a perspective view of a pulley assembly housing.

FIG. 7A illustrates a perspective view of a pulley assembly.

FIG. 7B illustrates a perspective view of a pulley assembly with a housing cover removed.

FIG. 8 illustrates an exploded view of the pulley assembly of FIG. 6.

FIG. 9 illustrates an exploded view of the pulley assembly of FIG. 6.

FIG. 10 illustrates an enlarged view of a rotational stop idler interfaced with a pulley housing cover.

FIG. 11 illustrates a cross-sectional view of the articulation knob and brake assembly.

FIG. 12 illustrates a perspective view of the articulation knob and brake assembly.

FIG. 13 illustrates a side view of the articulation knob and brake assembly.

FIG. 14 illustrates an exploded view of the articulation knob and brake assembly.

FIG. 15 illustrates a cross-sectional view of the up-down articulation knob and brake portion of the articulation knob and brake assembly FIG. 16 illustrates a perspective exploded view of the up-down articulation brake lever and up-down articulation brake outer housing.

FIG. 17 illustrates an exploded view of up-down articulation hub, the up-down articulation brake detent and the up-down articulation brake caliper.

FIG. 18 illustrates a perspective view of the up-down articulation brake outer housing and the up-down articulation shaft.

FIG. 19 illustrates a perspective view of the up-down articulation brake outer housing of FIG. 18.

FIG. 20 illustrates a side-elevational view of the articulation knob and brake assembly without the articulation knobs, outer housings, the up-down articulation brake lever and without the articulation brake inner housings.

FIG. 21 illustrates a perspective view of the left-right articulation brake outer housing and the left-right articulation shaft.

FIG. 22 illustrates a perspective view of the left-right articulation brake shaft and the left-right articulation brake hub.

FIG. 23 illustrates a perspective view of the left-right articulation brake shaft and left-right articulation brake hub of FIG. 22.

FIG. 24 illustrates a partially exploded view of the articulation knob and brake assembly with the up-down articulation knob, a left-right articulation knob and a left-right articulation brake knob exploded away from the assembly.

FIG. 25 illustrates a perspective view of a reusable hand-piece assembly of an endoscope assembly.

FIG. 26 illustrates an exploded view of the reusable hand-piece assembly of FIG. 25.

FIG. 27 illustrates a perspective view of an insertion tube assembly.

FIG. 28 illustrates a cross-sectional view of the insertion tube assembly of FIG. 27.

FIG. 29 illustrates a side view of an articulating link assembly.

FIG. 30 illustrates the endoscope assembly and the insertion tube.

FIG. 31 illustrates the endoscope assembly and the insertion tube.

FIG. 32 illustrates a partially exploded view of the single-use shaft assembly.

FIG. 33 illustrates a partially exploded view of the pulley assembly exposing the elevator control assembly.

FIG. 34 illustrates a perspective view of an insertion tube tip assembly.

FIG. 35 illustrates a partially exploded view of the insertion tube tip assembly of FIG. 34.

FIG. 36 illustrates a perspective view of the insertion tube tip assembly of FIG. 34 with the distal cap removed.

FIG. 37 illustrates a partially exploded view of an elevator assembly.

FIG. 38 illustrates a perspective view of an example of a cartridge and a console including a control valve assembly.

FIG. 39 illustrates a perspective view of the control valve assembly of FIG. 38

FIG. 40 illustrates an environmental view of a medical packaging design.

FIG. 41 illustrates an environmental view of the medical packaging design with a cover removed.

FIG. 42 illustrates an environmental view of the medical packaging design with multiple covers removed.

FIG. 43 illustrates an environmental view of the medical packaging design with a cartridge removed and secured to a console.

FIG. 44 illustrates an environmental view of the medical packaging design with a handle of the single-use shaft assembly removed and secured to a reusable hand-piece assembly.

FIG. 45 illustrates a perspective view of a packaging system for a medical product.

FIG. 46 illustrates a top view of the packaging system of FIG. 45.

FIG. 47 illustrates an exploded view of the packaging system of FIG. 45.

FIG. 48 illustrates a perspective view of the packaging system of FIG. 45 with a cover removed.

FIG. 49 illustrates a perspective view of the packaging system of FIG. 45 with a first and a second cover removed.

FIG. 50 illustrates a perspective view of another example of a packaging system for a medical product.

FIG. 51 illustrates a top view of the packaging system of FIG. 50.

FIG. 52 illustrates another perspective view of the packaging system of FIG. 50.

FIG. 53 illustrates a cartridge with a suction tube having a t-fitting.

FIG. 54 illustrates flowchart of a method of selecting between the imaging devices of one or more endoscopes.

FIGS. 55A-D illustrates one embodiment of an image sensor assembly.

FIGS. 56A-D illustrates various enlarged views of a camera and LED board of the embodiment shown in FIGS. 55A-D.

FIGS. 57A-E illustrates various views of an electrical connector of a cartridge of the embodiment shown in FIGS. 55A-D.

FIGS. 58A-D illustrates various views of a single-use shaft assembly electrical connector of the embodiment shown in FIGS. 55A-D.

FIGS. 59A-D illustrates a camera and LED board flex circuit and micro connector, as well as, a single-use shaft assembly electrical connector flex circuit and micro connector of the embodiment shown in FIGS. 55A-D.

FIGS. 60A-D illustrates a second embodiment of an image sensor assembly.

FIGS. 61A-D illustrates various enlarged views of a camera and LED board of the embodiment shown in FIGS. 60A-D.

FIGS. 62A-E illustrates various views of an electrical connector of a cartridge of the embodiment shown in FIGS. 60A-D.

FIGS. 63A-D illustrates various views of a single-use shaft assembly electrical connector of the embodiment shown in FIGS. 60A-D.

FIGS. 64A-D illustrates a camera and LED board flex circuit and micro connector, as well as, a single-use shaft assembly electrical connector flex circuit and micro connector of the embodiment shown in FIGS. 60A-D.

FIGS. 65A-C illustrates a third embodiment of an image sensor assembly.

FIGS. 66A-F illustrates various enlarged views of a camera and LED board of the embodiment shown in FIGS. 65A-C.

FIGS. 67A-E illustrates various views of an electrical connector of a cartridge of the embodiment shown in FIGS. 65A-C.

FIGS. 68A-B illustrates a camera and LED board flex circuit and micro connector of the embodiment shown in FIGS. 65A-C.

### DESCRIPTION OF THE SELECTED EXAMPLES

For the purpose of promoting an understanding of the principles of the disclosure, reference will now be made to the examples illustrated in the drawings and specific language will be used to describe the same. It will nevertheless be understood that no limitation of the scope of the disclosure is thereby intended. Any alterations and further modifications in the described examples, and any further applications of the principles of the disclosure as described herein are contemplated as would normally occur to one skilled in the art to which the disclosure relates. Exemplary examples of the disclosure is shown in greater detail, although it will be apparent to those skilled in the relevant art that some features that are not relevant to the present disclosure may not be shown for the sake of clarity.

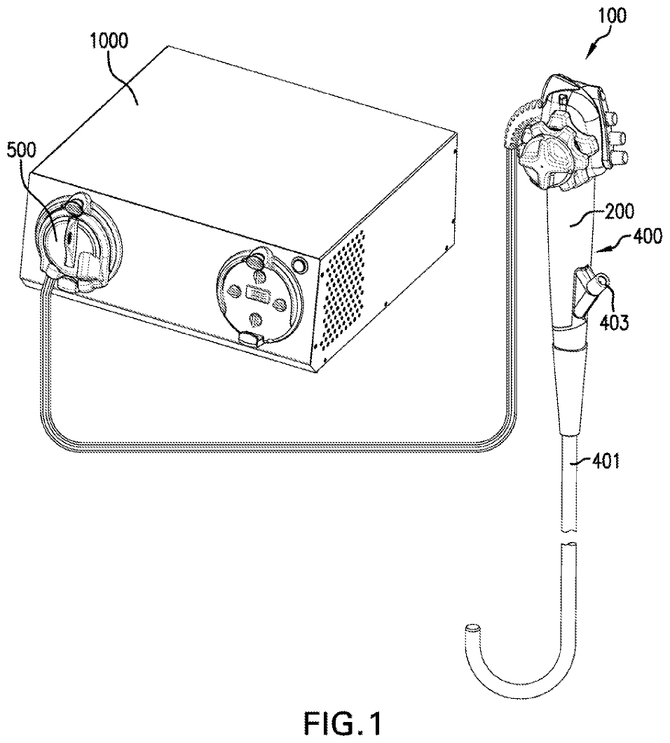

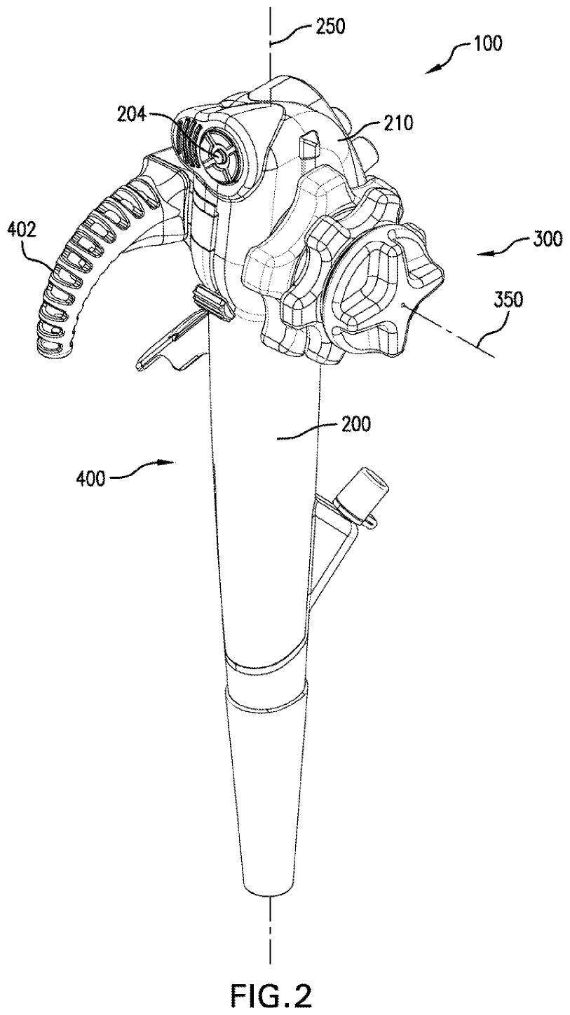

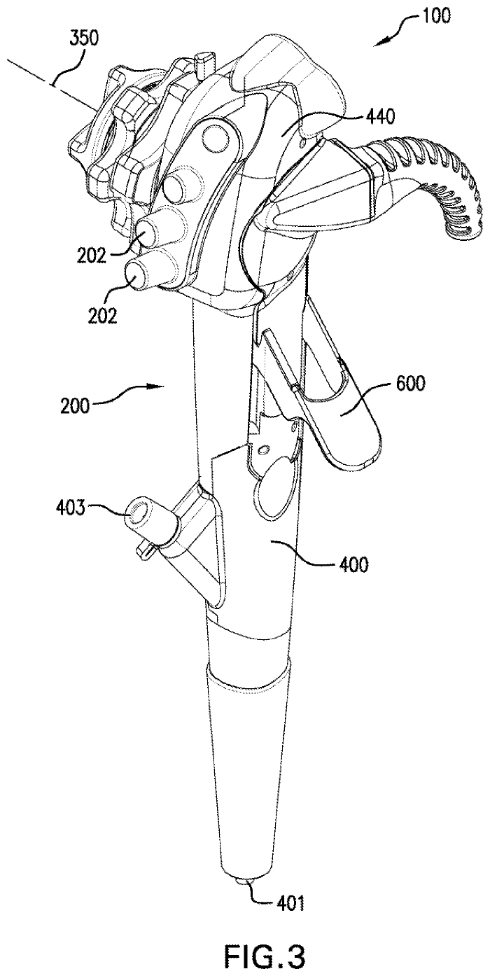

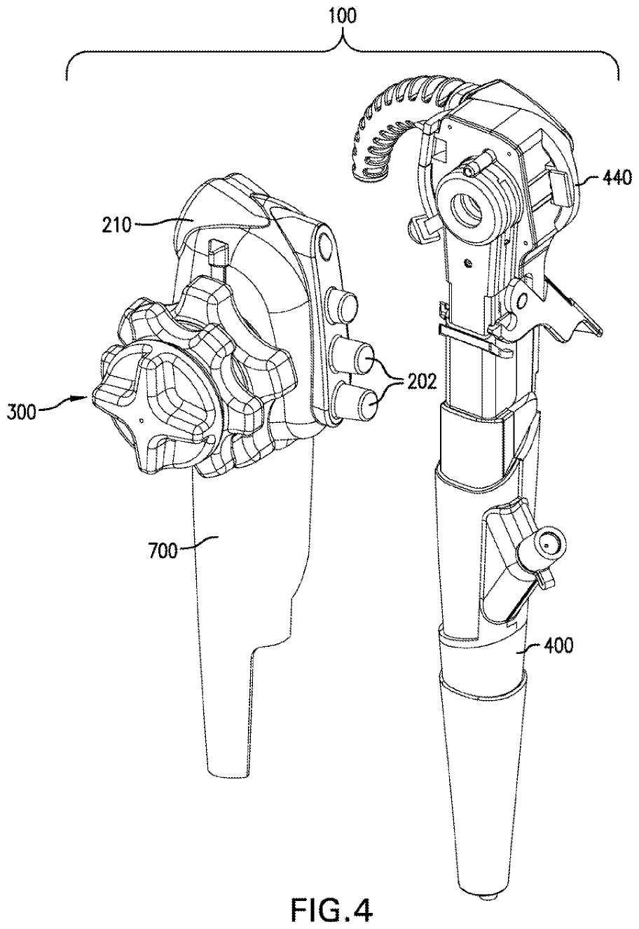

The present disclosure provides an endoscope assembly comprising a reusable hand-piece and a single-use shaft assembly. FIGS. 1-4 illustrate an endoscope assembly. FIG. 1 illustrates the endoscope assembly 100, cartridge 500 and a console 1000. More particularly, FIGS. 1-4 illustrate the endoscope assembly with the reusable hand-piece 200 attached to the single-use shaft assembly 400. FIG. 4 illustrates the endoscope assembly with the reusable hand-piece detached from the single-use shaft assembly.

The reusable hand-piece is selectively attachable and detachable from the single-use shaft assembly such that the reusable hand-piece may be used serially with a number of single-use shaft assemblies while the single-use shaft assembly, including the cartridges, can be discarded and/or reconditioned after a single use. The reusable hand-piece and/or single-use shaft assemblies may include a lock for selectively locking the reusable hand-piece and single-use shaft assembly to one another. A latch handle 600 (e.g., a lever) for actuating the latching mechanism connecting the reusable hand-piece to the single-use shaft assembly may be positioned on the reusable hand-piece and/or the single-use shaft assembly. Additional locking mechanisms such as magnets may be used in the alternative or concurrently.

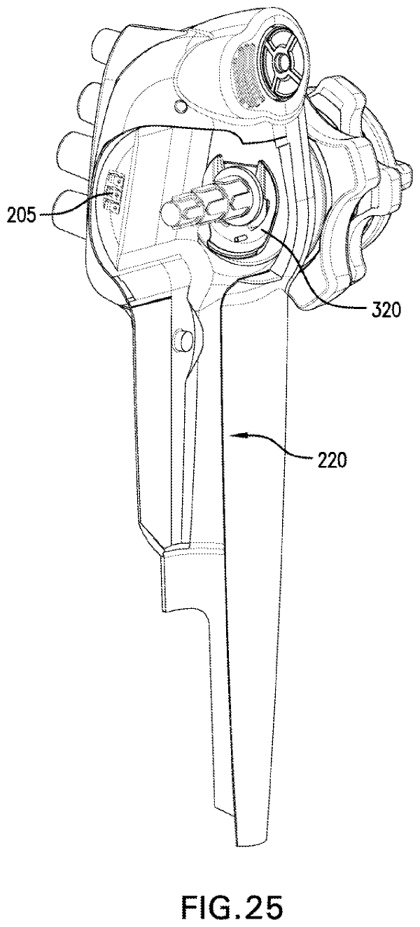

The reusable hand-piece can include a hand-piece housing 210 supporting an articulation knob and brake assembly 300; control switches 202 for operating light(s), such as light emitting diodes, and/or flush and/or aspiration fluid paths; one or more navigation switches 204 for controlling a console communicating with the endoscope assembly; and/or an electrical connector 205 (See FIG. 25) for electrically coupling the reusable hand-piece to the single-use shaft assembly.

The single-use shaft assembly can include an insertion tube 401 for insertion within the body of a patient, a tubing and electrical connector 402 connected to a console, an access port 403 for selectively accessing a lumen of the single-use shaft assembly (e.g., for taking a sample such as a biopsy) and/or for delivering a fluid through a lumen of the single-use shaft assembly, and/or an electrical connector 404 for electrically coupling the single-use shaft assembly to the reusable hand-piece.

A tubing and/or electrical connector 402, sometimes referred to as an “umbilical”, extends from the endoscope assembly to a cartridge that is connectable to a console suitable for use with the endoscope assembly. The tubing and/or electrical connector can extend from the single-use shaft assembly and/or from the reusable hand-piece. In some embodiments, the umbilical may directly connect to the console, eliminating the for a cartridge.

A console may provide electrical power, fluids and/or fluid pressure, vacuum pressure, and/or send and/or receive electrical signals with the endoscope assembly. The console may include a monitor (not shown) for visualizing signals received from the endoscope assembly such as a video feed from a camera at the distal end of the single-use shaft assembly.

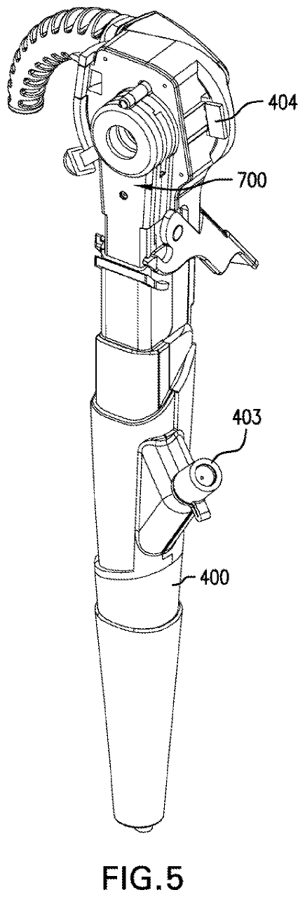

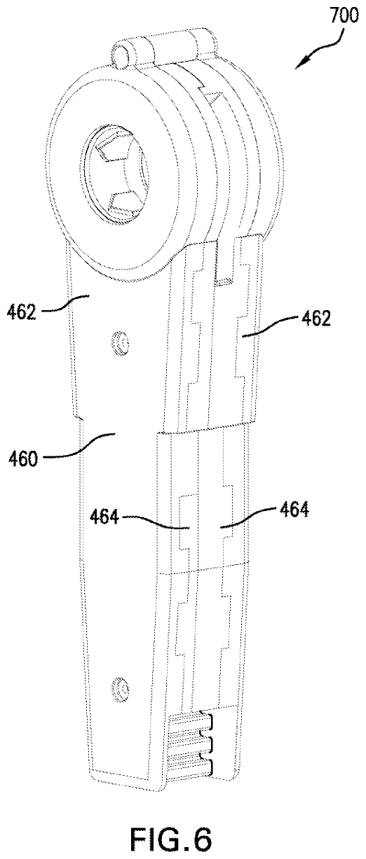

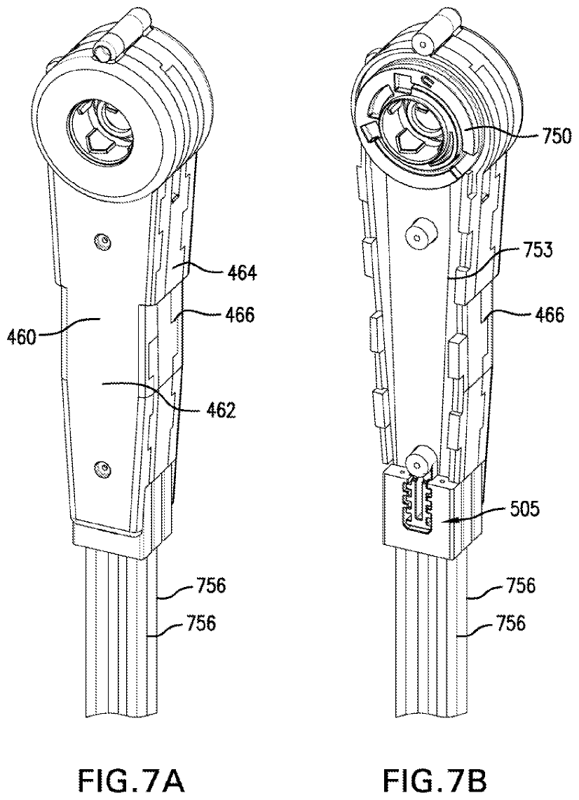

The single-use shaft assembly can include one or more pulley assemblies 700, such as those shown in FIGS. 5-7B. Pulley assemblies can include at least one pulley 750 arranged to receive an end of an articulation shaft. Preferably, rotation of the articulation knob associated with the articulation shaft rotates the at least one pulley. Preferably, the at least one pulley and end of the articulation shaft have a geometry (e.g., a mating feature) for rotationally coupling the pulley and articulation shaft to one another. Preferably, the mating feature does not rely solely on friction between the articulation shaft and pulley. Preferably, the mating features include a geometric interference between the articulation shaft and pulley.

Associated with the at least one pulley is an articulation wire 753 or a pair of articulation pull wires (e.g., two separate wires or one continuous wire looped over the pulley and having wire segments extending from opposing sides of the pulley) configured to bend a distal shaft section of the single-use shaft assembly upon actuation. The articulation wires may be fixed (e.g., tied or fixed by anchors (not illustrated)) to the pulley and/or extend around at least a portion of the pulley (e.g., loop around the pulley). Preferably, the one or more pulley assemblies include two pulleys (one for each articulation wire) and articulation wires (at least one for each pulley). In the illustrated example, each pulley includes one groove (two total) for the articulation wires. Alternatively, each pulley may include two grooves (four total) with only one wire per groove to reduce the chance the wires overlap and the friction from the overlap affecting performance. Preferably, the articulation wires are configured to bend the distal shaft section in orthogonal planes.

The one or more pulleys and associated one or more articulation wires may be contained within a pulley housing 460. The pulley housing may be constructed of housing portions, such as cover pieces 462 and inner pieces 464. The housing portions may cooperate to at least partially enclose the one or more pulleys.

The pulley housing may be constructed in a sandwich-like fashion with a pair of cover pieces on the outside/ends and a pair of inner pieces coupled on one side to one cover piece each and on the other side to each other. In this embodiment, the pulley housing is configured to house at least two pulleys. Such housing portions may cooperate to enclose the pulley and associated articulation wire(s). Preferably the housing portions resist the articulation wire(s) from separating from the pulley(s). The pulley housing can defines openings for receiving articulation shafts and/or a brake shaft of the articulation control assembly.

Preferably, the housing portions do not contact one another along a purely linear butt joint. For example, the inner pieces and/or cover pieces may contact along a rectilinear path. In some aspects, the inner pieces and cover pieces are constructed with a castellation 466 along the joint between the two pieces and down the length of the pieces so that the castellation on the inner piece matches with and couples to the castellation on the cover piece. Advantageously, avoiding a purely linear butt joint can assist in preventing wires from escaping between portions of the pulley assembly. In other examples, an intersection between housing portions may be linear, curved, curvilinear, rectilinear, or some other desired arrangement.

In addition, the pulley assembly may include an articulation coil termination block 505 that receives one or more articulation coils 756 wrapping around respective articulation pull wires connected to the pulleys. The articulation coil termination block is adjustable to remove articulation pull wire slack. In other words, the articulation coil termination block is adjustable along the longitudinal axis of the endoscope assembly to control the articulation pull wire tension.

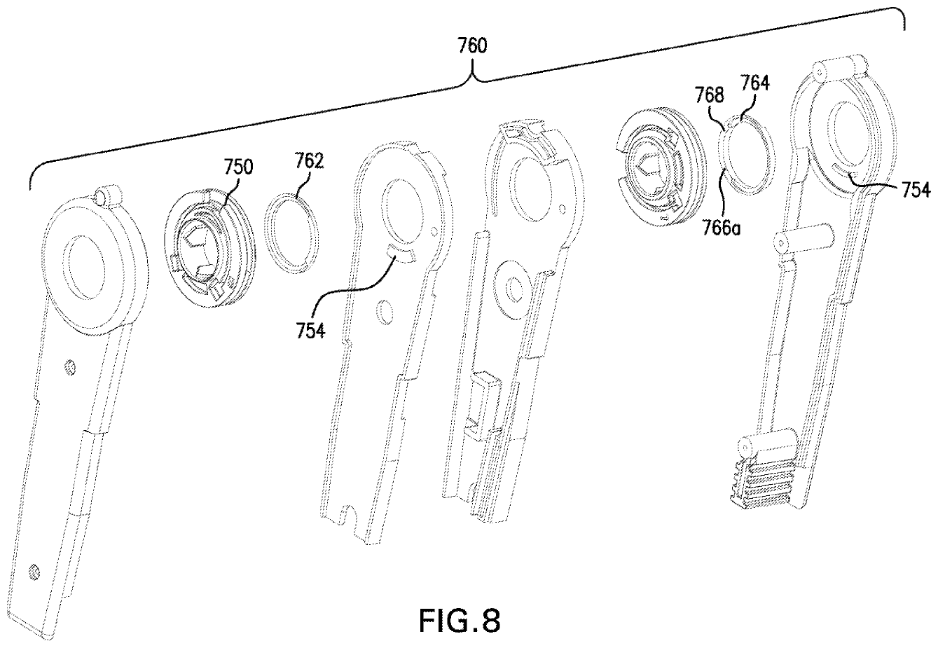

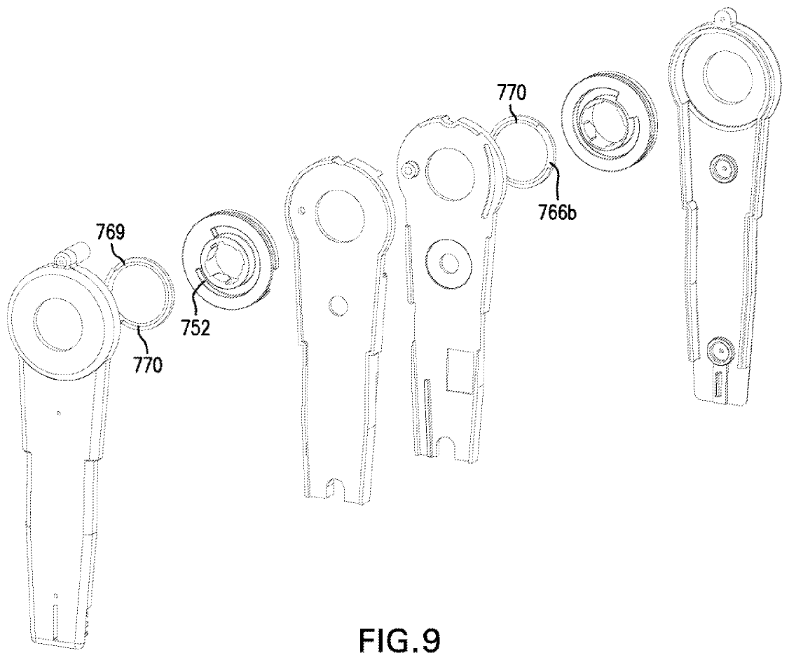

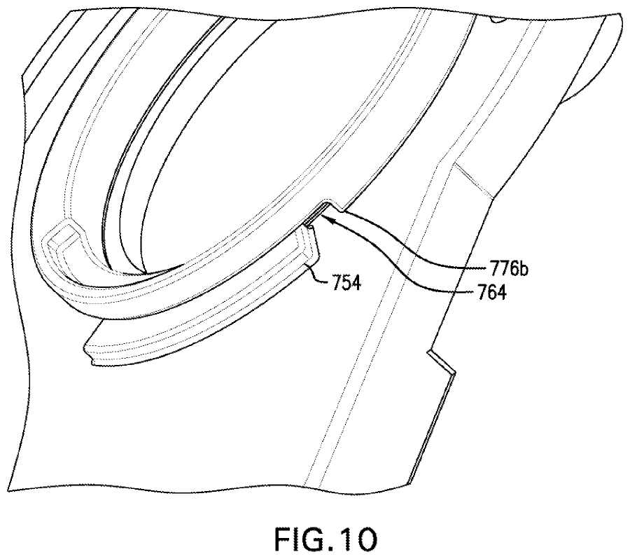

Illustrated in FIGS. 8-10 is a “two stage articulation limit.” The two stage rotational stop assembly 760 allows the articulation knobs to rotate further than with a single stage stop. The two stage rotational stop assembly may be completely enclosed within the pulley housing. The two stage rotational stop assembly may include two pulleys and two rotational stop idlers 762 located on a side of each pulley, such as the side opposite a side having a recess for an articulation wire anchor. One pulley for the up-down articulation and the other for the left-right articulation. Each rotational stop idler may include a separate groove 764 (two total) around the circumference of each sides of the rotational stop idler. The groove terminates at a stop 766a (e.g., a protrusion), preventing the groove from circling three hundred and sixty degrees around the rotational stop idler.

Each pulley includes a boss 752 on the side of the pulley that interfaces with the groove on a first side 768 of a rotational stop idler. During rotation of the pulley relative to the rotational stop idler, the boss on the pulley follows the groove of the idler until it is blocked by the stop on the rotational stop idler. Once the boss on the pulley contacts the stop, the force of the pulley's rotation is transferred to, and may rotate, the idler. Rotation of the idler is permitted depending on the contact of the rotational stop idler and the housing.

On a second side 769 of the rotational stop idler, a second stop 766b interfaces with a boss 754 located on the pulley housing to limit the degree of rotation of the rotational stop idler relative to the housing. For example, during rotation of the idler relative to the housing, the boss on the housing follows the groove 766 on the second side of the rotational stop idler until the second stop member of the idler contacts the boss on the housing, thus stopping the rotation of the idler relative to the housing. Accordingly, after rotation of the pulley relative to the rotational stop idler is prevented in a first direction, and after rotation of the rotational stop idler relative to the housing is prevented in the first direction, rotation of the articulation knob and pulley relative to the housing in the first direction is also prevented. The pulley may be rotated in a second direction opposite of the first direction. The explanation for rotation in the first direction is applicable to the second direction.

To prevent the rotation stop idler from wobbling, rotational stop idler may include a continuous bearing surface 770 on both sides of the idler. The continuous bearing surface provides a stable base to keep the idler balanced as it rotates within and against the pulley housing.

Advantageously, this example has the ability to limit the articulation knob rotation for an endoscope when the articulation knob rotation required exceeds the rotation limits that can be achieved with a single stage stop. On one side of the rotational stop idler, the allowed angular rotation of the pulley relative to idler is equal to the angular length of the groove of the idler minus the angular length of the boss on the pulley. On the other side of the rotational stop idler, the rotation allowed of the idler relative to the housing is equal to the angular length of the groove of the idler minus the angular length of the boss on the pulley housing. These two “stages” and degrees of rotation, when added together, provide the total angular distance the pulley (and/or the articulation knob) may travel relative to the housing. For demonstrative purposes only, if the bosses have an angular length of forty-five degrees each and the grooves of the rotational stop idler each have an angular length of three-hundred fifteen degrees, the degrees of rotation on each side of the idler would equal two hundred and seventy degrees and the sum of the two sides would be five hundred and forty angular degrees of freedom of the pulley relative to the housing. It is understood that the bosses, grooves, and/or stop members may have the same or different angular lengths and that the bosses, grooves, and/or stop members may have angular lengths greater or less than forty-five degrees.

It is also anticipated that one or more of the bosses may be a separate component and that the grooves may follow a spiral path. Accordingly, as the boss follows the path of the groove, the boss may translate along a surface of pulley and/or housing. Advantageously, such arrangements can provide for grooves having an angular length of three hundred sixty degrees or greater.

While the grooves and stops have been described as being defined by the rotational stop idler and the bosses have been described as being of the pulley and/or housing, the arrangements disclosed herein are not so limited. It will be appreciated that the grooves and stops may be defined by the pulley and/or housing and the one or more bosses may be a portion of the rotational stop idler.

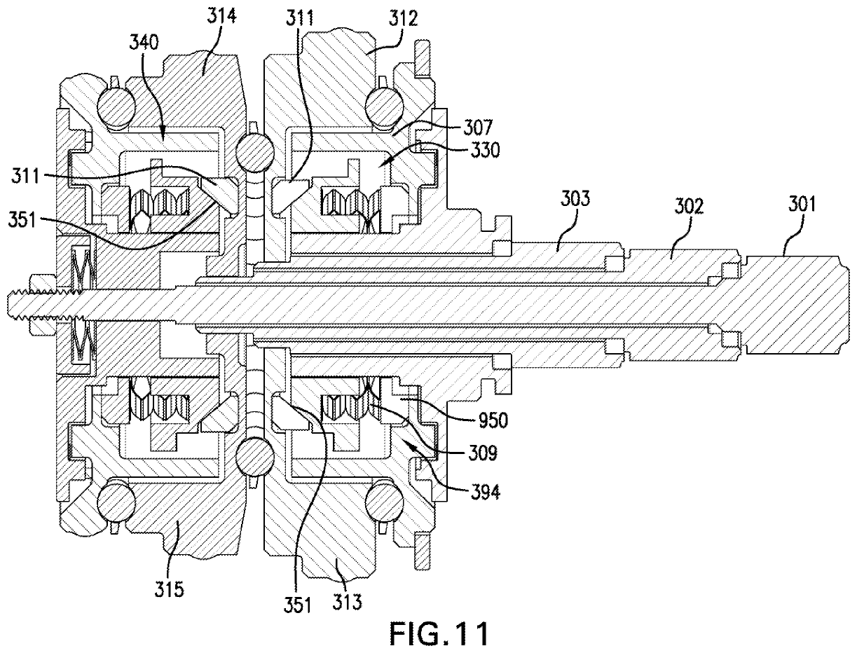

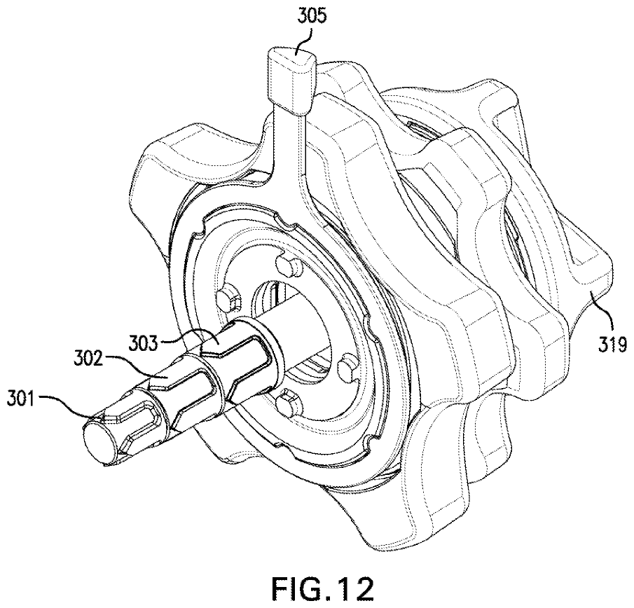

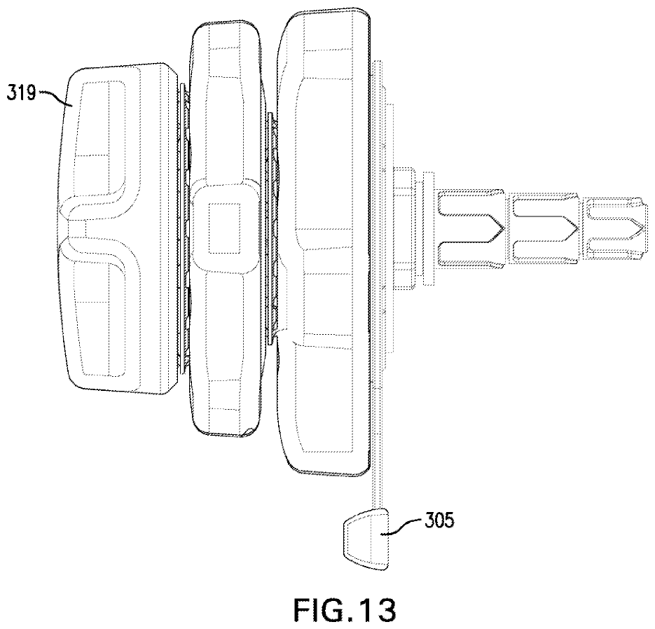

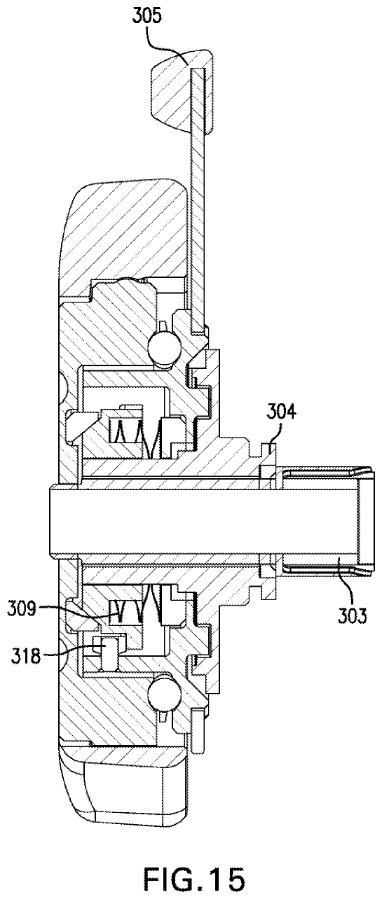

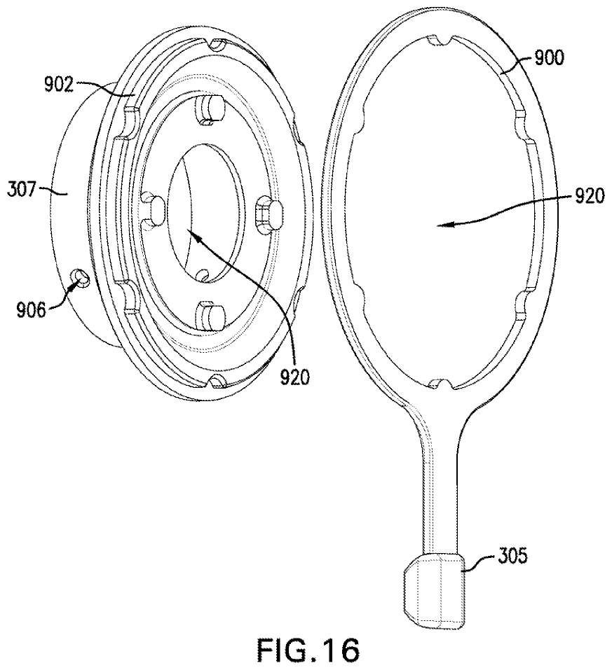

With exemplary reference now to FIGS. 11-23, the articulation knob and brake assembly of the reusable hand-piece can include an up-down articulation knob 313, a left-right articulation knob 314, an up-down articulation brake 330 and a left-right articulation brake 340. During operation, when the reusable hand-piece is attached to the single-use shaft assembly, articulation of the up-down articulation knob articulates a distal portion of the single-use shaft assembly in a first plane and actuation of the left-right articulation brake knob actuates the distal portion of the single-use shaft assembly in a second plane transverse to the first plane. Preferably, the second plane is orthogonal to the first plane.

When the reusable hand-piece is coupled to the single-use shaft assembly, actuation of the up-down articulation brake lever engages the up-down articulation brake that resists rotation of the up-down articulation knob relative to a portion of the endoscope assembly, such as the housing of the reusable hand-piece. Actuation of the left-right articulation brake knob 319 engages the left-right articulation brake that resists rotation of the left-right articulation knob relative to a portion of endoscope assembly, such as a housing 440 of the single-use shaft assembly.

The reusable hand-piece and the single-use shaft assembly are preferably configured to mate with one another. For example, the reusable hand-piece assembly may define a recess 220 arranged to receive a portion of the single-use shaft assembly, or vice versa. In many examples, the recess of the reusable hand-piece assembly arranged to receive a portion of the single-use shaft assembly will be on a side of the reusable hand-piece assembly opposing the side having the knobs of the articulation knob and brake assembly. One of the reusable hand-piece assembly or the single-use shaft assembly may surround at least a portion of the other, partially and/or completely, on two or more opposing sides. In other embodiments, one of the reusable hand-piece assembly or the single-use shaft assembly may enclose the other.

A portion of the single-use shaft assembly may be receivable in the reusable hand-piece, or vice versa, along a direction parallel-to or transverse to a longitudinal axis of the reusable hand-piece or single-use shaft assembly. For example, the reusable hand-piece may receive the single-use shaft assembly along a direction orthogonal to a longitudinal axis 250 of the reusable hand-piece. In many instances, when joining the reusable hand-piece and single-use shaft assembly to one another, the single-use shaft assembly will move, relative to the reusable hand-piece, along a direction parallel to a rotation axis 350 of the articulation knob and brake assembly.

When the reusable hand-piece assembly is attached to the single-use shaft assembly, the articulation knob and brake assembly cooperates with the single-use shaft assembly to articulate a distal segment of the single-use shaft assembly and/or resist rotation of one or more articulation knobs relative to the endoscope assembly. For example, as discussed in more detail elsewhere herein, one or more articulation shafts 302, 303 and/or brake shafts 301 of the articulation knob and brake assembly may engage the pulley assembly and/or housing of the single-use shaft assembly. One or more shafts of the articulation knob and brake assembly extend through the pulley assembly and engage the housing of the single-use shaft assembly on an opposing side of the pulley assembly from the articulation knob and brake assembly.

In many examples a portion of the articulation knob and brake assembly extends into the recess of the reusable hand-piece arranged to receive a portion of the single-use shaft assembly.

Regarding the up-down articulation control by the articulation knob and brake assembly, the up-down articulation knob is supported on an up-down articulation brake outer housing 312. Positioned within the up-down articulation brake outer housing is an articulation brake inner housing 307, an articulation brake position detent 308, an articulation brake spring 309, an up-down articulation brake caliper 310, and an angled articulation brake friction disc 311. The angled surface 351 of the articulation brake friction disc may be non-perpendicular to the axis of rotation.

Connected to the articulation brake inner housing is the up-down articulation brake lever. Extending through the up-down articulation brake lever is an up-down articulation hub 304 and an up-down articulation shaft. The up-down articulation hub, which is positioned within the articulation brake inner housing, supports the articulation brake position detent and the up-down articulation brake caliper. Rotation of the up-down articulation hub is fixed relative to the housing of the reusable hand-piece which, in turn, fixes rotation of the articulation brake position detent and the up-down articulation brake caliper relative to the housing of the reusable hand-piece as well.

Upon rotation of the up-down articulation brake lever relative to the up-down articulation hub, the articulation brake inner housing rotates relative to the articulation brake position detent and the up-down articulation brake caliper. Upon rotation in a first direction, pins 318 extending from the up-down articulation brake inner housing towards the up-down articulation brake caliper engage angled surfaces 360 of the up-down articulation brake caliper to axially move the articulation brake caliper towards the articulation brake spring to compress the articulation brake spring. Upon rotation in a second direction, the pins move along the angled surfaces to allow the articulation brake spring to force the up-down articulation brake caliper towards the angled surface of the articulation brake friction disc and the up-down articulation brake outer housing to compresses the articulation brake friction disc between the up-down articulation brake caliper and the up-down articulation brake outer housing. This contact between the stationary (relative to the reusable hand-piece) up-down articulation brake caliper, the angled surface of the articulation brake friction disc, and the up-down articulation brake outer housing results in resistance of the up-down articulation knob, which is supported by the up-down articulation brake outer housing, rotating relative to the reusable hand-piece. In other examples, the articulation brake surface may be perpendicular to the axis of rotation. Advantageously, angled brake surfaces can increase the braking force in comparison to the perpendicular brake surface at equivalent applied spring force.

As rotation of the up-down articulation brake position detent is fixed relative to the housing of the reusable hand-piece via the up-down articulation brake hub, rotation of the articulation brake inner housing relative to the housing also rotates the articulation brake inner housing relative to the up-down articulation brake position detent. The up-down articulation brake position detent may be arranged to retain the up-down articulation brake inner housing in one or more positions. For example, the detent may retain the up-down articulation brake inner housing in a first position in which braking resistance is applied via up-down articulation brake friction disc interaction between the up-down articulation brake caliper and the up-down articulation brake outer housing and/or in a second position one in which braking resistance is not applied. The brake inner housing and/or detent may be arranged for retaining the up-down articulation brake inner housing in any of a plurality of positions to provide an adjustable level of rotational resistance to the articulation control (e.g., articulation control knobs).

Extending through the center of the up-down articulation brake hub is an up-down articulation shaft. The up-down articulation shaft has a first end 370 rotationally fixed relative to the up-down articulation brake outer housing. A second end 372 of the up-down articulation shaft communicates with the pulley assembly of the single-use shaft assembly. For example, the second end of the up-down articulation shaft may be attached to the up-down articulation pulley such that rotation of the up-down articulation shaft rotates the up-down articulation pulley. In this way, rotation of the up-down articulation knob rotates the up-down articulation brake outer housing which rotates the up-down articulation shaft which rotates the up-down articulation pulley which actuates the up-down articulation wire(s).

Regarding left-right articulation, the structure and operation of the left-right articulation knob and brake assembly is the same as the up-down articulation knob and brake assembly with the exception that rotation of the left-right articulation brake hub 317 is fixed relative to the housing of the single-use shaft assembly and that such rotation is fixed via a left-right articulation brake shaft extending through the left-right articulation shaft. Similar to the up-down articulation knob and brake assembly, the left-right articulation knob is supported by a left-right articulation brake outer housing 315 which contains an angled articulation brake friction disc 311, a left-right articulation brake caliper 316, an articulation brake spring, an articulation brake position detent and an articulation brake inner housing.

Extending through the left-right articulation brake knob and the left right articulation brake outer housing is the left-right articulation shaft. The left-right articulation shaft has a first end 380 rotationally fixed relative to the left-right articulation brake outer housing and a second end 382 engaging the pulley assembly of the single-use shaft assembly (e.g., the second end rotationally fixed to the left-right articulation pulley) such that rotation of the left-right articulation brake knob rotates the left-right articulation brake outer housing which rotates the left-right articulation brake shaft which in turn rotates the left-right articulation pulley which in turn translates the left-right articulation wire(s).

The left-right articulation brake shaft, which extends through the left-right articulation shaft, has a first end 390 rotationally fixed to left-right articulation brake hub and a second end 392 rotationally fixed relative to the housing of the single-use shaft assembly when the reusable hand-piece assembly is attached to the single-use shaft assembly. Similar to the operation of the up-down articulation knob and brake assembly, rotation of the articulation brake inner housing relative to the left-right articulation brake caliper moves the left-right articulation brake caliper towards or away from the left-right articulation brake outer housing and the angled surface of the brake friction disc positioned therebetween. The left-right articulation brake knob is rotationally fixed to the left-right articulation brake inner housing such that rotation of the left-right articulation brake knob rotates the left-right articulation brake inner housing relative to the left-right articulation brake hub, the left-right articulation brake caliper, and the left-right articulation brake position detent. Accordingly upon engagement of the left-right articulation brake, the left-right articulation brake caliper and angled surface of the brake friction disc resist rotation of the left-right articulation brake outer housing and left-right articulation knob relative to the left-right articulation brake shaft and the single-use shaft assembly.

The second end of the left-right articulation brake shaft may be arranged to slidably engage with the housing of the single-use shaft assembly and rotationally fix the left-right articulation brake shaft relative thereto. Positioned circumferentially around the left-right articulation brake shaft is the left-right articulation shaft with its second end arranged to engage the left-right articulation pulley cooperating with the left-right articulation wire(s). And, positioned circumferentially around the left-right articulation shaft is the up-down articulation shaft having its second end arranged to engage the up-down articulation pulley associated with the up-down articulation wire(s).

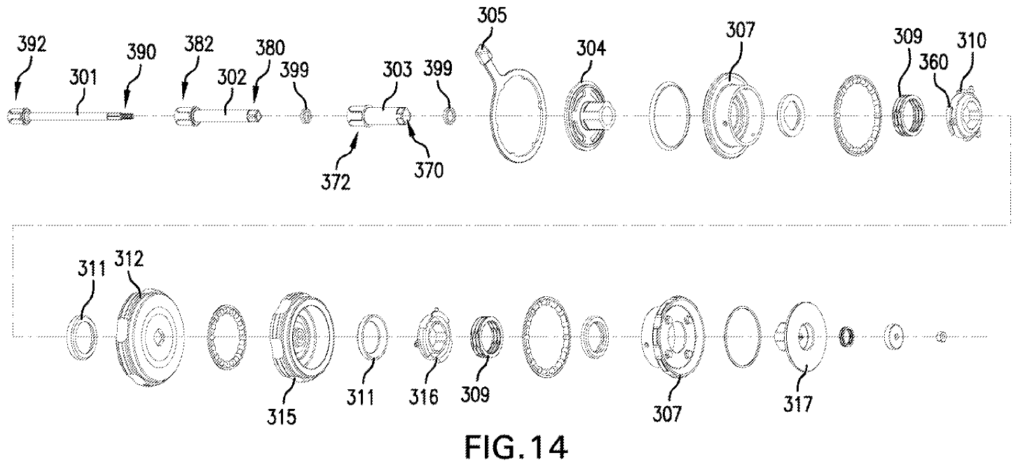

As shown in FIG. 14, the articulation knob and brake assembly may include a plurality of O-rings between the various components when assembled. Also illustrated are ball bearings disposed within the assembly to reduce the friction created during rotation.

With reference now to FIG. 16, the up-down articulation brake lever can have a surface 900 configured to mate with a surface 902 of the up-down articulation inner housing such that rotation of the up-down articulation brake lever rotates the up-down articulation brake inner housing relative to the housing of the reusable hand-piece assembly. Preferably, the geometry of surface interferes with the geometry of surface 902 during rotation. The up-down articulation brake inner housing can define one or more apertures 906 arranged to receive transverse pins that engage the angled surfaces of the up-down articulation brake caliper. Additionally, an inner surface 910 of the up-down articulation brake inner housing can include recesses or protrusions arranged to engage detent spring (not shown). The up-down articulation brake lever and the up-down articulation brake inner housing define an aperture 920 for receiving the up-down articulation hub.

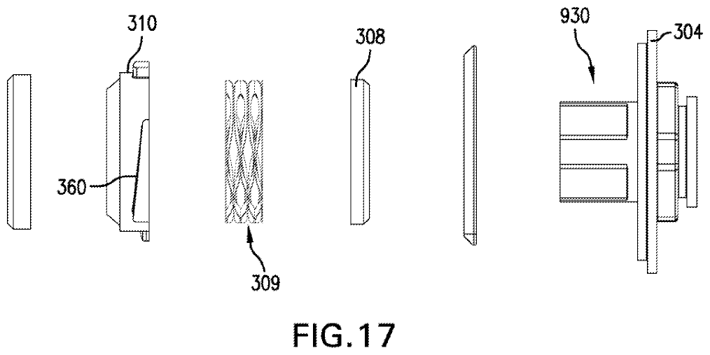

Turning now to FIG. 17, the up-down articulation hub has a portion 930 that extends through the up-down articulation brake position detent and the up-down articulation brake caliper. Preferably portion, the up-down articulation brake position detent, and/or the up-down articulation brake caliper have a cross-sectional geometry that fixes the up-down articulation brake detent, the up-down articulation brake caliper, and/or the up-down articulation brake hub from rotation relative to one another.

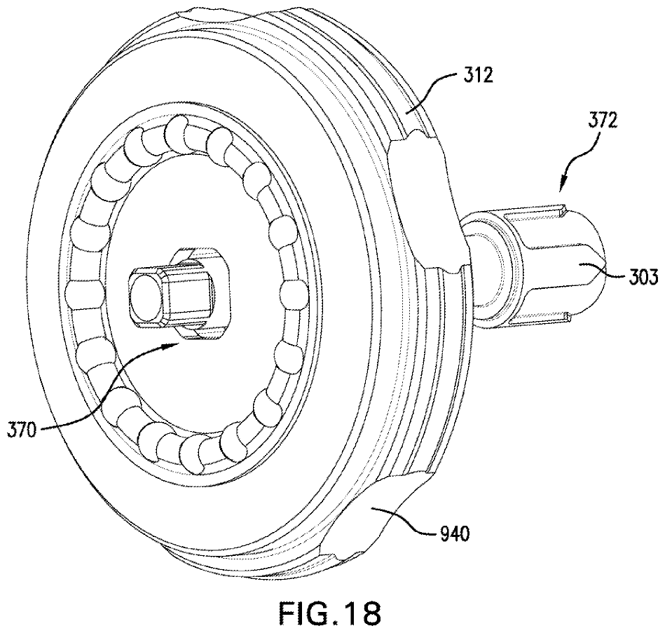

Turning now to FIG. 18, the up-down articulation brake outer housing is shown engaged with and rotationally fixed relative to the up-down articulation shaft. The up-down articulation brake outer housing has a portion 940 arranged to rotationally fix the up-down articulation knob thereto (e.g., via recesses arranged to receive protrusions of the up-down articulation knob or vice versa).

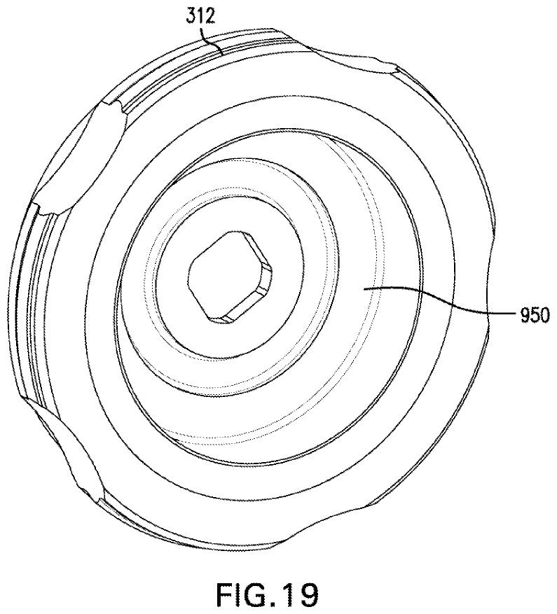

Turning now to FIG. 19, the inside of the up-down articulation brake outer housing is shown as having an inner friction surface 950 arranged to engage the angled/tapered surface of the up-down articulation brake friction disc when the up-down articulation brake is engaged.

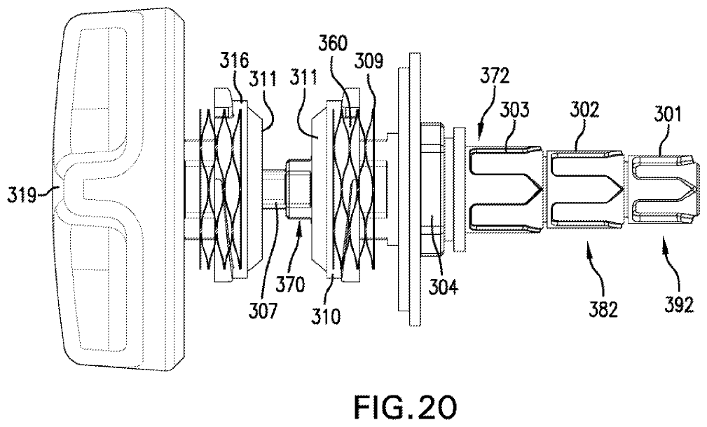

Turning now to FIG. 20, when the up-down articulation brake lever is rotated relative to the housing of the reusable hand-piece, the up-down articulation brake inner housing rotates with the up-down articulation brake lever and moves pins extending from the apertures of the up-down articulation brake inner housing relative to the up-down articulation brake caliper. The pins move along the sloped surfaces of the up-down articulation brake caliper to translate the up-down articulation brake caliper along the up-down articulation hub in cooperation with the up-down articulation brake spring. When configuring the up-down articulation brake from a disengaged configuration to an engaged configuration, the up-down articulation brake caliper forces the up-down articulation brake friction disc against the up-down articulation brake outer housing (compressing the up-down articulation brake friction disc). When configuring the up-down articulation brake from an engaged configuration to a disengaged configuration, the up-down articulation brake caliper moves away from the up-down articulation brake housing and the up-down articulation brake friction disc and compresses (e.g., further compresses) the up-down articulation brake spring. FIG. 11 shows the up-down articulation brake in the disengaged configuration with a gap 394 between the up-down articulation brake friction disc and the inner friction surface 950 of the up-down articulation brake outer housing.

The up-down articulation brake inner housing may have detent recesses or protrusions to engage one or more protrusions or recesses of the up-down articulation brake position detent. Such an arrangement may provide securement of the up-down articulation brake lever in one or more positions and/or provide a tactile and/or audible indication of the up-down articulation brake entering and/or leaving the one or more configurations or positions.

The left-right articulation knob and brake assembly work in a similar fashion to the up-down articulation knob and brake assembly with the exception of the left-right articulation brake hub being rotatably secured relative to the housing of the endoscope assembly by a left-right articulation brake shaft extending centrally through the left-right articulation shaft and the up-down articulation shaft.

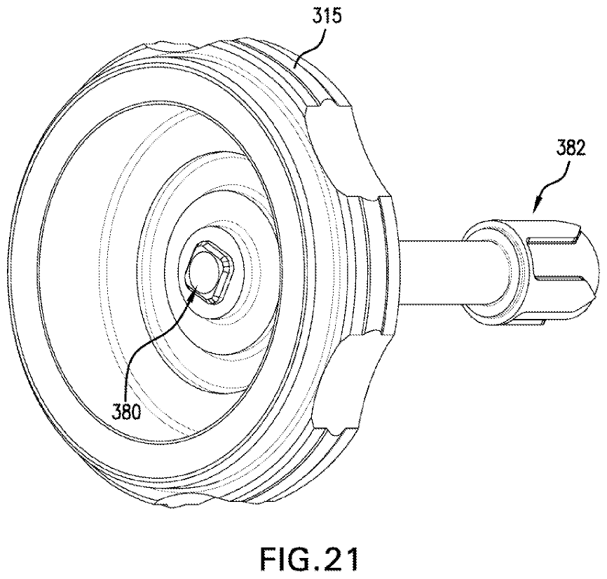

With reference to FIG. 21, the left-right articulation brake outer housing and the left-right articulation shaft are shown assembled and rotationally fixed relative to one another.

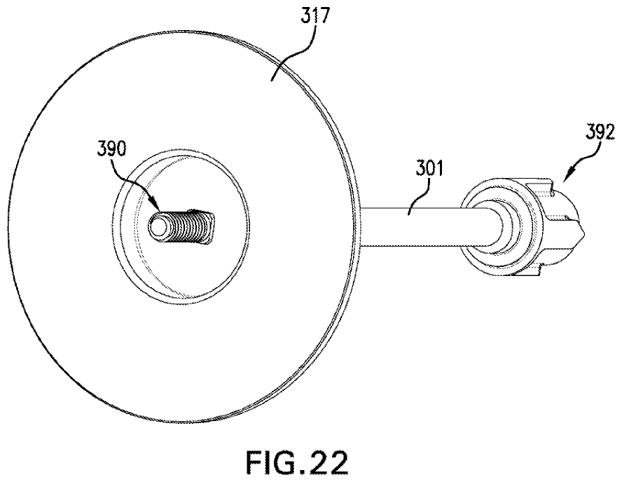

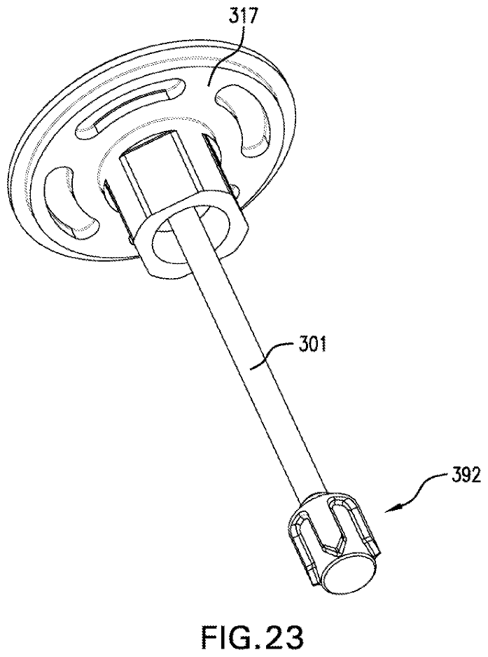

FIGS. 22 and 23 illustrate the left-right articulation brake shaft in the left-right articulation brake hub. As can be seen along the left-right articulation brake shaft has a first end received within and rotationally fixed to the left-right articulation brake hub. A second end of the left-right articulation brake shaft is arranged to engage the housing of the single-use shaft assembly during attachment of the reusable hand-piece assembly and the single-use shaft assembly and rotationally fixe the left-right articulation brake shaft relative to the single-use shaft assembly. Accordingly, when the reusable hand-piece assembly is attached to the single-use shaft assembly, the left-right articulation brake hub is rotationally fixed relative to the housing of the single-use shaft assembly.

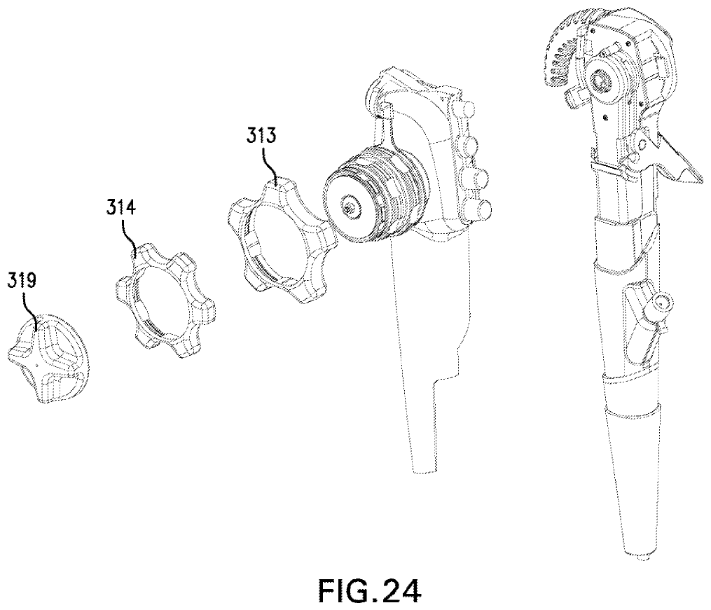

Turning to FIGS. 24-26, the up-down articulation knob 313, the left-right articulation knob 314 and the left-right articulation brake knob 319 may be detachable (or “snap off”) from the rest of the articulation knob and brake assembly. As discussed above, the up-down articulation brake outer housing has a portion arranged to rotationally fix the up-down articulation knob thereto (e.g., via recesses arranged to receive protrusions of the up-down articulation knob or vice versa). Similarly, the left-right articulation brake outer housing is arranged to fix the left-right articulation knob in the same fashion. The recesses and protrusions arrangement allow the up-down articulation knob, left-right articulation knob and left-right articulation brake knob to be easily snapped off or snapped on to the articulation knob and brake assembly. Advantageously, the snap off feature allows the knobs to be removed to facilitate cleaning of the reusable hand-piece between procedures.

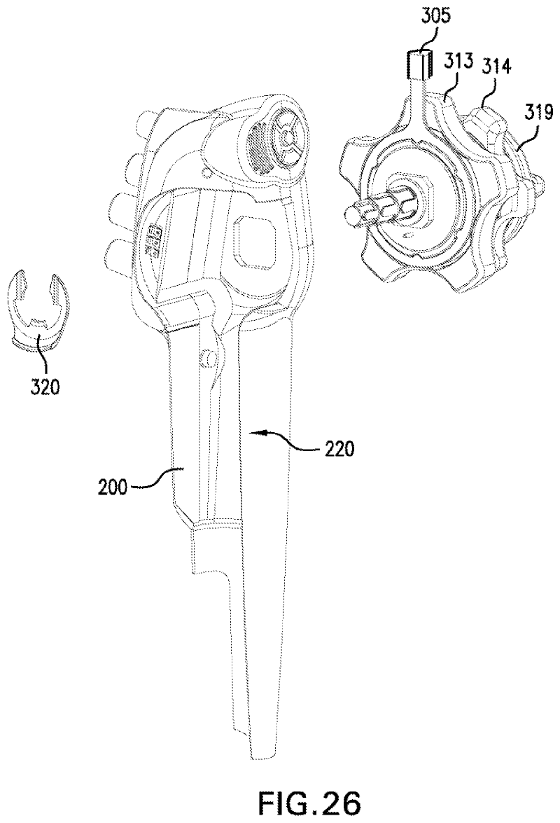

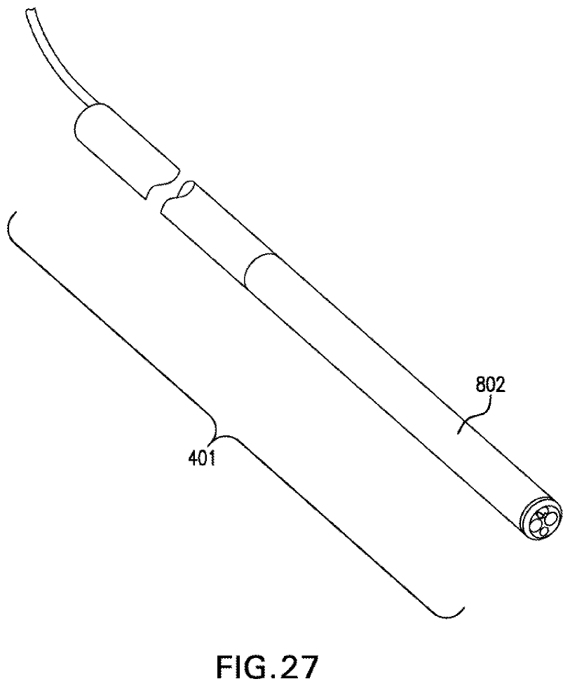

As illustrated in FIGS. 25 and 26, the reusable hand-piece may include a retention clip 320. Retention clip is used to secure articulation knob and brake assembly to the reusable hand-piece. The retention clip may be coupled with the up-down articulation hub to secure the articulation knob and brake assembly. In between procedures, if necessary to facilitate cleaning, the retention clip may be removed from the articulation and brake assembly, separating the articulation and brake assembly from the housing of the reusable hand-piece. Once cleaning is complete, the articulation and brake assembly may be reattached to the housing using the retention clip.

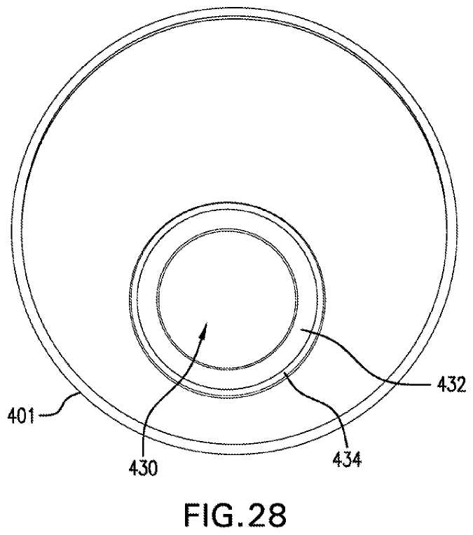

Turning to FIGS. 27 and 28, the insertion tube may include an instrument tube 437 extending within the interior of at least a portion of the insertion tube. The instrument tube defines a tool channel 430 allowing various instruments to be inserted and extended through the insertion tube. The instrument tube may be created through a coextrusion process. The inner extrusion 432 may be comprised of High Density Poly Ethylene (HDPE) for lubricity. The outer extrusion 434 may be formed of polyvinyl chloride (PVC) or polylactic acid (PLA) to facilitate bonding.

The insertion tube may include an optical sensor module for providing readable information by the console and/or project an image on a monitor such as a video feed from the end of the insertion tube. This video feed can be infrared, thermal or visible light. The insertion tube 401 may include a light-emitting diode (LED) wire for providing power to an LED at the end of the insertion tube.

The insertion tube may include fluid paths discussed herein defined by tubes, such as a suction tube, tissue irrigation tube, camera flush tube, and/or insufflation tube. The irrigation tube allows the user to clear the visual field of any debris during use of the instrument. Often, this involves the removal of excess blood so the place of interest may be visualized. The suction tubing allows for the aspiration of particulate matter or various secretions from inside the patient's body. The camera flush tube and insufflation tube may be combined by a Y fitting. Various combinations of the tubing are envisioned by the applicant to minimize the number of tubes required in the insertion tube.

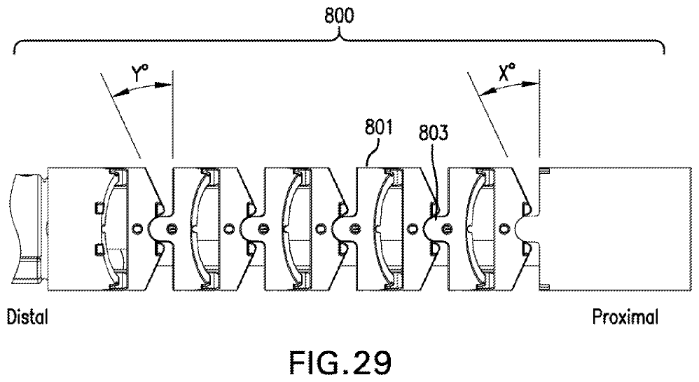

FIG. 29 depicts an example of an articulating joint assembly 800 located in a flexible articulation section 802 of the insertion tube. The articulating joint assembly may comprise a proximal end and a distal end. Configured between the proximal end and the distal end are a plurality of connected articulation links 801. Each link has a contact point 803 where the link is connected to the adjacent link. Adjacent links are arranged to articulate relative to one another. For example, adjacent articulation links may be arranged to pivot an angle relative to one another. For example, adjacent links may be arranged to pivot an angle of 2x relative to one another, where angle (x) is measured between contact points of each adjacent link when the links are parallel to one another. Adjacent articulation links in the articulating joint assembly may be pivotable to different degrees than other adjacent articulation links in the articulating joint assembly. Preferably, adjacent articulating links may be formed so that the angles of articulation are different between different pairs of the articulation links. Advantageously, such an arrangement may be used to obtain a variable radius along the length of the flexible articulation section during articulation. Pairs of adjacent links having different angles of articulation may be used to create a variable curve along the length of the flexible articulation section.

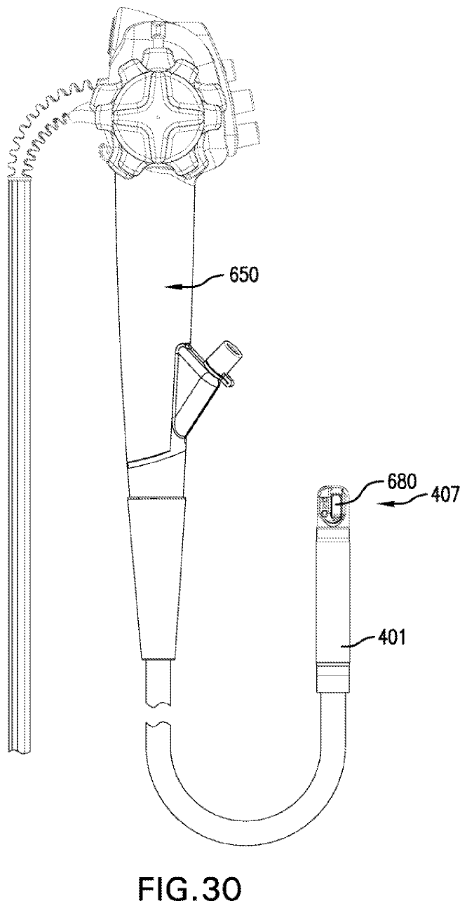

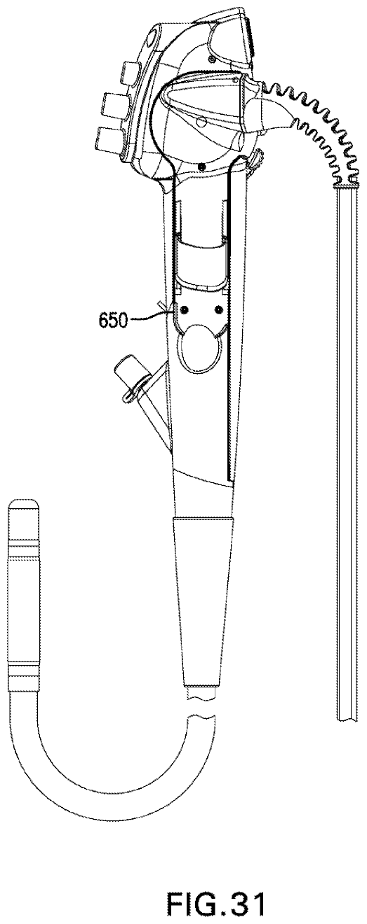

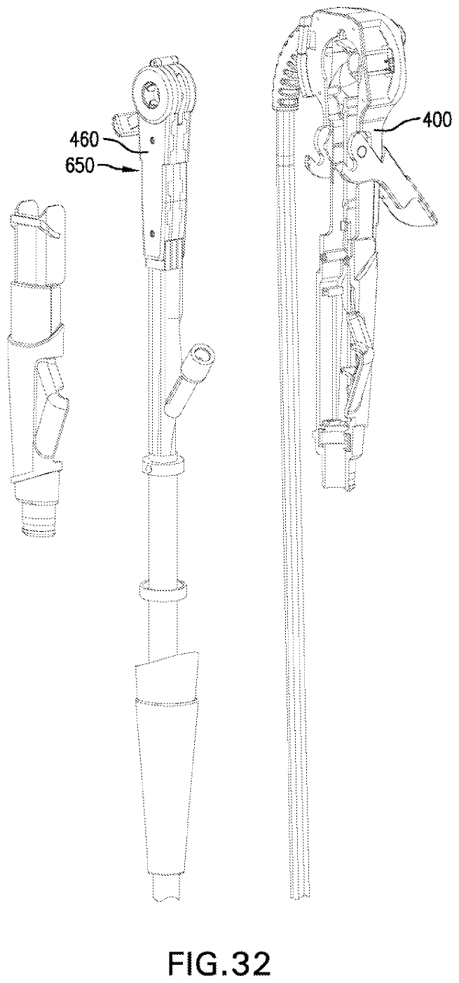

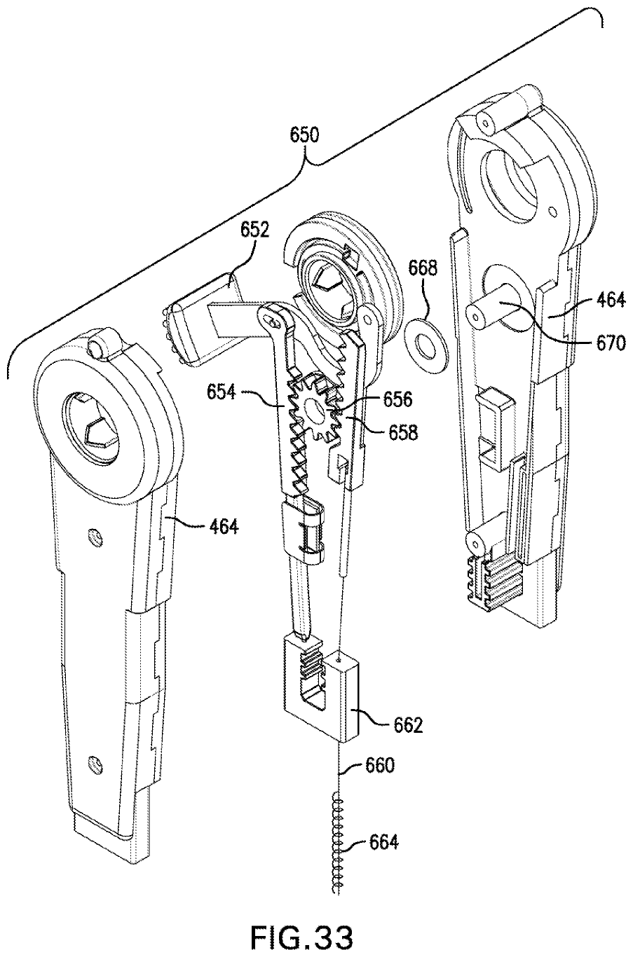

Regarding FIGS. 30-33, illustrated is an elevator control assembly 650 for adjusting the height of an elevator 680 at a distal end 407 of the insertion tube. The elevator control assembly may be disposed between the two inner pieces of the pulley housing. The elevator control assembly includes an elevator control lever 652. The elevator control lever articulates around the articulation shafts via pins constrained by curvilinear tracks. The elevator control lever being operationally coupled to an elevator driving rack 654. The elevator driving rack is coupled to an elevator pinion 656. The elevator pinion is also operationally coupled to an elevator driven rack 658. The elevator driven rack is coupled to an elevator articulation control wire 660. In addition, the elevator control assembly may include an elevator control coil termination block 662 that receives one or more elevator control coils 664 wrapping around respective elevator articulation control wires connected to the elevator driven rack.

Articulation of the elevator control lever operates the elevator driving rack, which in turn rotates the elevator pinion. When the elevator pinion is rotated, it drives the elevator driven rack to articulate an elevator articulation control wire with elector control coils wrapped around the wire. For example, actuating the elevator control lever in one direction causes the elevator height to increase, while articulation in the opposing direction causes the elevator height to decrease.

In order to maintain the position of the elevator control lever when user input is not provided, the elevator control assembly utilizes a compressed o-ring 668. The o-ring is secured around a protrusion 670 extending from one of the housing covers. The o-ring is pressed against the elevator control pinion, maintaining the position of the control lever utilizing the friction created between the o-ring and the elevator control pinion. In other examples, the position of the lever is maintained utilizing friction created by a spring element.

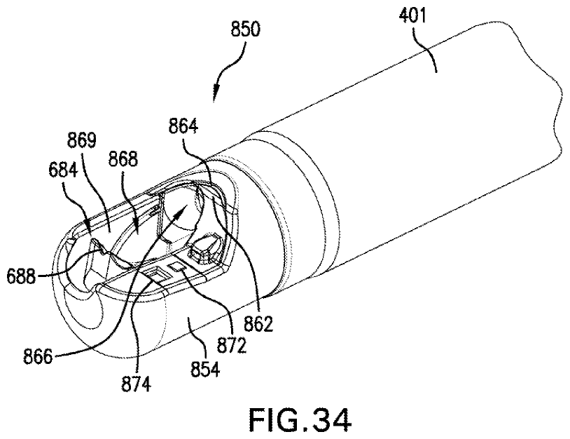

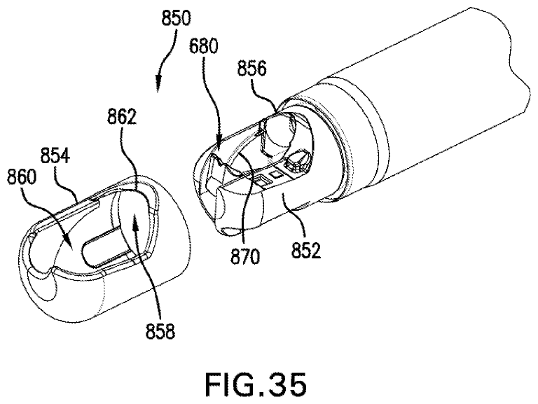

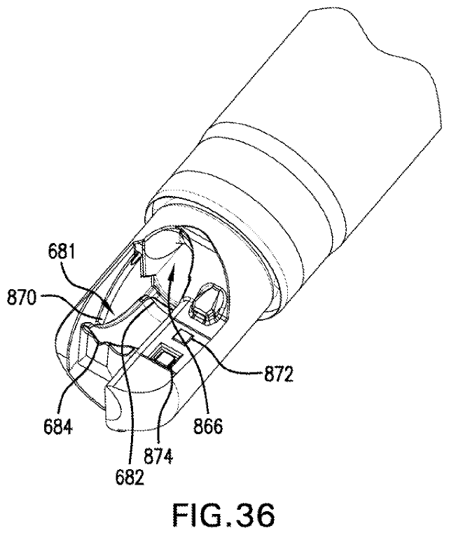

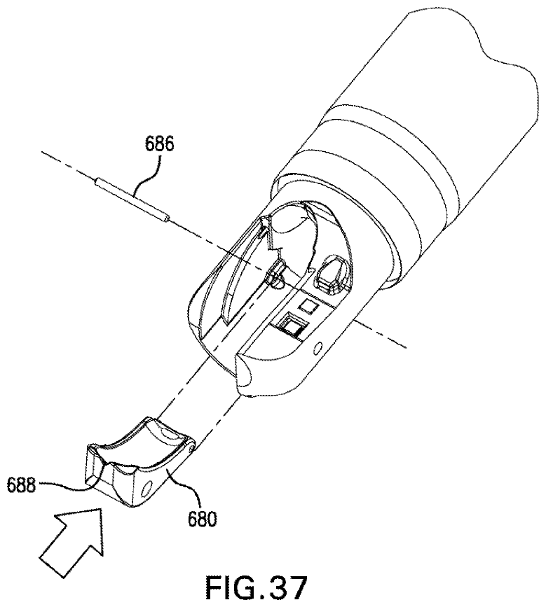

Illustrated in FIGS. 34-37 is an insertion tube tip assembly 850. The insertion tube tip assembly includes the insertion tube, a distal elevator link 852, a distal cap 854 and an elevator assembly 681. The insertion tube includes distal end 407. The distal end includes a distal tip 856 including the distal elevator link, distal cap and the elevator assembly.

The distal cap includes a first opening 858 allowing the cap to slide over the elevator assembly. The distal cap further includes a second opening 860 to allow instruments to extend through and exit the distal cap and operate within the patient's body. The distal cap further includes a notch 862 on a top portion 864 of the distal cap. The distal cap may be removable from the elevator assembly and/or distal portion of the insertion tube. In some examples, the distal cap is not included.

The elevator assembly includes an instrument tube opening 866 to extend instruments out of the instrument tube. The instrument tube 437 may extend through a portion of the insertion tube. The opening opens into a working chamber 868. The chamber may be defined by opposing side walls 869, a distal surface of the insertion tube and an elevator. Adjacent to the insertion tube opening is the elevator 680. The elevator may be used to adjust the elevation of an instrument within the patient's body to access the desired location within the body during a procedure. The elevator may include a proximal end 682 and a distal end 684. The elevator may be secured to the elevator assembly by a pivot pin 686. During articulation of the elevator, the elevator rotates around the pivot pin, allowing the distal end of the elevator to be deflected up or down. Deflecting the elevator allows a user to concurrently deflect an instrument. The elevator may include a notch 688 at the distal end of the elevator opposing the notch of the distal cap. The opposing notches may be utilized to limit unintended longitudinal or lateral movements of instruments passing through the instrument channel and into the patient's body. Notch 688 may hold an instrument protruding out of opening to increase the control of the instrument.

The elevator assembly further includes an elevator control wire 870. Elevator control wire may be a single strand pre-formed wire for articulation of the elevator. The elevator wire may translate over a static curved ramp resulting in a rotational torque on the elevator. Elevator wire is manipulated by the elevator control assembly to control the height of the elevator.

The elevator assembly may further define a light window 872 through which a light may be admitted, such as from a light emitting diode. This arrangement allows the user to illuminate a patient's body during a procedure. The elevator assembly may further define an imaging window 874 to provide images back to the endoscopist.

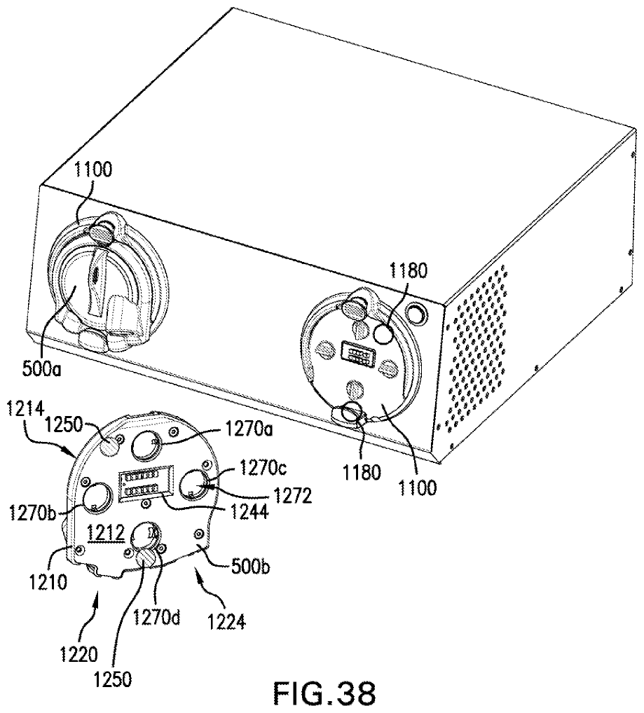

The present disclosure further provides a system comprising an endoscope assembly, a cartridge and a console. FIG. 38 generally illustrates the system operationally connected for use, preferably by a medical professional. The system may include the cartridge inserted into a control valve assembly 1100 of the console. The cartridge can be connected to the endoscope by the umbilical. For instance, the cartridge may be connected to a single-use shaft assembly of the endoscope by one or more fluid tubes and/or electrical conductors.

The cartridge may be connected to support equipment. The support equipment may include an irrigation pump, an insufflator, and/or a vacuum pump (e.g., stand-alone or central). The cartridge may be positioned between the endoscope and the support equipment.

In some examples, the console may include a user interface (not shown) allowing the user to alter the various variables of the endoscope system. In one example, the user interface may be a touchscreen integrated into an electric visual display. In another example, the user interface could include a keyboard, mouse, trackball, and/or touch sensitive pointing device, etc.

The console may include a control valve assembly for receiving and actuating upon a cartridge. The console may provide electrical power, fluids and/or fluid pressure, vacuum pressure, and/or send and/or receive electrical signals with the endoscope assembly. The console may include a monitor (not shown) for visualizing signals received from the endoscope assembly such as a video feed from a camera at the distal end of the single-use shaft assembly.

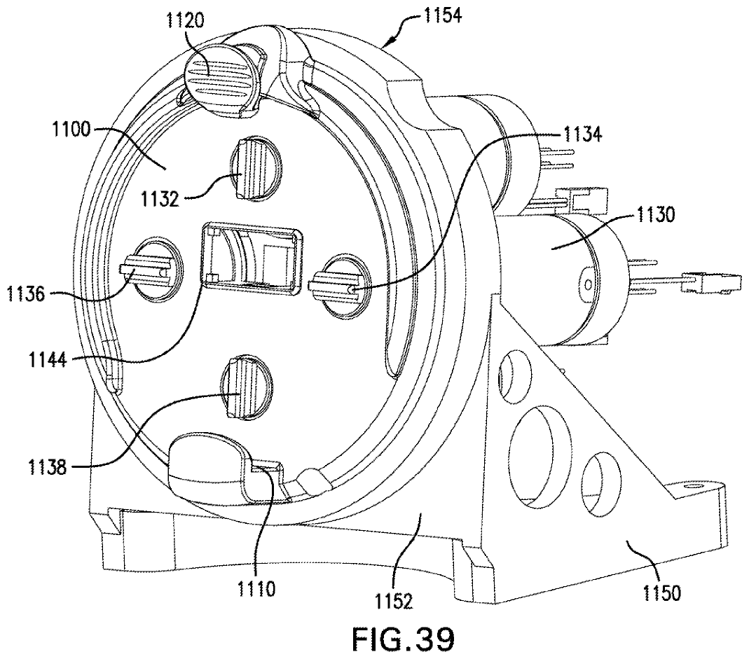

With reference to FIG. 39, the control valve assembly is shown in greater detail. The control valve assembly may be used to selectively actuate an actuatable portion of fluid paths of the cartridge. The control valve assembly may include actuators 1130 and a support structure 1150 comprising a first surface 1152 and a second surface 1154.

The actuator may be a linear actuator or rotational actuator. The actuator may be a solenoid. Although as few as one actuator is appreciated by the applicant, in preferred examples the control valve assembly includes four actuators. The actuators are arranged to actuate valve portions of fluid paths in the cartridge to selectively open and/or close the fluid paths.

The valve portions of the fluid paths may comprise any acceptable valve. For example, the valve portion may comprise a petcock. The valve portion may also comprise a portion of tubing arranged for compression (e.g., a pinch valve) between the anvil on the first surface of the door and the actuator (e.g., solenoid) of the control valve assembly. The valve portions may serve as a valve for any number of fluid functions of the endoscope, such as a suction valve associated with actuator 1132, a camera flush valve associated with actuator 1134, an insufflation valve associated with actuator 1136, and/or an insufflation vent valve associated with actuator 1138.

The actuators of the control valve assembly include electrical connectors to be received by the console and electrically connect the actuators to the console. When connected to the console, the console can provide electrical power and user inputted commands to the actuator to open and/or close the fluid pathways of the cartridge.

The cartridge may include an identifier readable and/or writable by the control valve assembly. The identifier may include information regarding the type of cartridge and/or usage information (e.g., whether the cartridge has been used before). Preferably, the control valve assembly is configured to read and process the identifier of the cartridge (e.g., to determine the type of cartridge and/or whether the cartridge has been used previously). The identifier may be readable and/or writable through an/the electrical connector of the cartridge and/or through other means (e.g., RFID, optics, and/or mechanical contact).

The first surface of the support structure may include the electrical connector for forming electrical connections to the electrical connector of the cartridge. The electrical connection between the control valve assembly of the console and the cartridge may allow the console to receive images and control data (valve/actuator and/or image data) passed from the endoscope through the cartridge to the console. The electrical connection may allow the console to supply power to the light-emitting diode (LED) of the endoscope assembly.

The cartridge can be a single-use cartridge. The single-use cartridge can negate the need for the use of permanent valve bodies contained within a reusable endoscope and/or the console. This advantageously eliminates the need to sterilize reusable valves after every use.

The cartridge has a housing 1210. The housing can include a first side 1212 and a second side 1214. The housing further comprises a first fluid side 1220 and a second fluid side 1224 allowing for fluid to travel in and out of the cartridge housing. The housing may define one or more fluid pathways, fluid connector, and/or electrical connectors. For example, cartridge housing can include electrical connector 1244 in electrical communication with an electrical conductor of the endoscope and optionally an optical sensor module that contains optical sensor calibration data or a unique identifier for the endoscope. The housing may define windows 1270a-d. In one example, the housing includes four windows. The windows may be defined by inner surfaces of the housing. The inner surface may create a generally circular opening in the housing. The circumference of one window may be larger than the other windows to receive a larger fluid path cross section.

The windows can be configured to receive an actuator from the console into a chamber 1272 defined by the inner surface. As described above, the actuator may pass through the window and/or contact an anvil on the door of the control valve assembly of the console to create a pinch valve with the door. When the cartridge is positioned on the control valve assembly and the door is closed, the windows align with the anvils on the door and the actuators of the control valve assembly. The windows may be arranged to define a geometric shape. For instance, as illustrated, the windows may define a diamond.

As described above, one or more actuators may be solenoids configured to extend into the windows of the cartridge housing and/or contact the anvils on the door of the control valve assembly creating a pinch valve when closed.

The housing may further include an electrical connector for connecting to the electrical connector of the control valve assembly. The electrical connector of the cartridge may be spring finger electrical contacts configured to contact a corresponding number of flat pad electrical contacts on the control valve assembly of the console or vice versa. The electrical connector of the cartridge housing may pass images and control data provided by the endoscope to the console. The electrical connector of the housing may pass power from the console to the LED used by the endoscope in the patient's body.

Looking to FIG. 38, a view of the cartridge inserted into the control valve assembly of the console is shown. As can be seen, the cartridge is inserted between the first surface of the support structure of the control valve assembly. The first fluid side of the cartridge may include loose tubing that continues into the umbilical and to the single-use shaft assembly of the endoscope (not shown).

The second fluid side of the cartridge may be connected to support equipment. The support equipment may include an irrigation pump, an insufflator, and/or a vacuum pump (stand-alone or central). The cartridge may provide for fluid communication between the endoscope and the support equipment.

The control valve assembly may include a ledge surface 1110 and a latch 1120. The cartridge may include a ledge surface and a latch receiving portion. In alternate examples, the control valve assembly may include the latch receiving portion and the cartridge may include the latch. When the cartridge is received by the control valve assembly the latch contacts the latch receiving portion to secure a first end of the cartridge to the control valve assembly. Separately, and opposite of the latch and latch receiving portion, the ledge surface of the cartridge and the ledge surface of the control valve assembly contact each other to secure a second end of the cartridge to the control valve assembly.

In some examples, the cartridge may include two or more magnets 1250 located on the first side of the cartridge. In some examples, one magnet may be located at the bottom of the first side of the cartridge and the second magnet located at the top of the first side of the cartridge. The first surface of the control valve assembly may include two or more magnetic sensors 1180 (hall or reed), mirroring the locating of the magnets on the cartridge. For example, one located at the bottom of the first surface and the other located at the top of the first surface. The bottom magnet on the cartridge interfaces with the bottom magnetic sensor on the control valve assembly triggering the actuator (e.g., solenoid) valves to retract reducing the force required to tilt the cartridge into the control valve assembly. The top magnet interfaces with the top magnetic sensor on the control valve assembly to confirm the presence of the cartridge on the console, triggering the console to release the appropriate actuators and initiate the actuator operation mode for endoscopy.

FIG. 38 illustrates the console with a first cartridge 500a, a second cartridge 500b. The first cartridge may be connected to a first endoscope (not shown) and the second cartridge may be connected to a second endoscope (not shown). The first cartridge and the second cartridge may be securely inserted into a first control valve assembly and a second control valve assembly of the console, respectively. Although the console is illustrated with the first and second control valve assembly, the console may have any number control valve assemblies each capable of receiving an independent cartridge. The first control valve assembly may receive the first cartridge and the second control valve assembly may receive the second cartridge. It is not necessary for every control valve assembly to receive a cartridge during every medical procedure.

As illustrated, each cartridge may be connected to a separate set of support equipment and an independent medical device, such as an endoscope.

The first control valve assembly and the second control valve assembly are configured to operate independently and/or in a main/secondary communication configuration. The first or mother endoscope may be the main controller and the second or daughter endoscope may be the secondary controller. In this arrangement, the controls of the main endoscope may control the fluid functions (e.g., image controls and/or LED) of one or more secondary endoscopes. The main endoscopes may control the function of the fluid paths of the secondary endoscopes by providing an electrical signal to the second control valve assembly to selectively actuate an actuator as described herein (e.g., through one or more cartridges connected to the console). The first endoscope may further control a function of the second endoscope (e.g., operation of the LED and/or camera) by providing an electrical signal from the first endoscope to alter a condition of the second control valve assembly (e.g., alter an electrical signal provided by the second control valve assembly to the cartridge of the second endoscope). For instance, the control valve assembly may provide an electrical signal to the LED and/or the camera of the second endoscope based on the signal received from the first endoscope. This arrangement further allows for multiple endoscopes to be setup and utilized in a procedure at once, eliminating or reducing extensive setup time typically used to change one endoscope out for another endoscope and/or eliminating or reducing the need for additional equipment, such as multiple control consoles.

The console is illustrated with a touchscreen user interface. The user interface may be any suitable input/output device.

The single-use shaft assembly can include an insertion tube, an umbilical connected to the cartridge and therefore the console, an access port for selectively accessing a lumen of the single-use shaft assembly (e.g., for taking a sample such as a biopsy) and/or for delivering a fluid through a lumen of the single-use shaft assembly, and/or an electrical connector for electrically coupling the single-use shaft assembly to the reusable hand-piece.

The umbilical can extend from the cartridge to the single-use shaft assembly of the endoscope. Extending within the umbilical to the distal tip of the insertion tube are several fluid paths and/or electrical wires. The fluid paths may include paths for suction, tissue irrigation, camera flush, and/or insufflation. The fluid pathways can be free of valves from the distal tip of the endoscope to the end of the umbilical. The fluid pathways may include valves only within the boundary of the cartridge.

Preferably, one or more fluid paths are defined by monolithic tubing extending from the single use shaft assembly to the cartridge. More preferably, the monolithic tubing extends through the cartridge and out of the second fluid side of the cartridge.

The camera flush fluid pathway and insufflation fluid pathway may be combined by a T-fitting in the single-use shaft assembly into a combined camera flush and insufflation fluid pathway. This allows the pressure from insufflation to be used to push water from a water source through the camera flush fluid pathway and out the distal end to clean the camera lens of debris collected during a procedure.

The umbilical may further include an image data conductor, control data conductor, and/or an LED power conductor. The image data conductor and/or LED power conductor may extend through the single-use shaft assembly to the distal tip of the insertion tube. The image data conductor may allow the transmission of the image data taken from the camera back to the console. The LED power conductor may provide power from the console to the LED at the distal tip of the insertion tube, thus providing illumination for the procedure. The control data conductor allows the communication of control data from the controls of the reusable hand piece to the cartridge which, in turn, allows communication to the console.

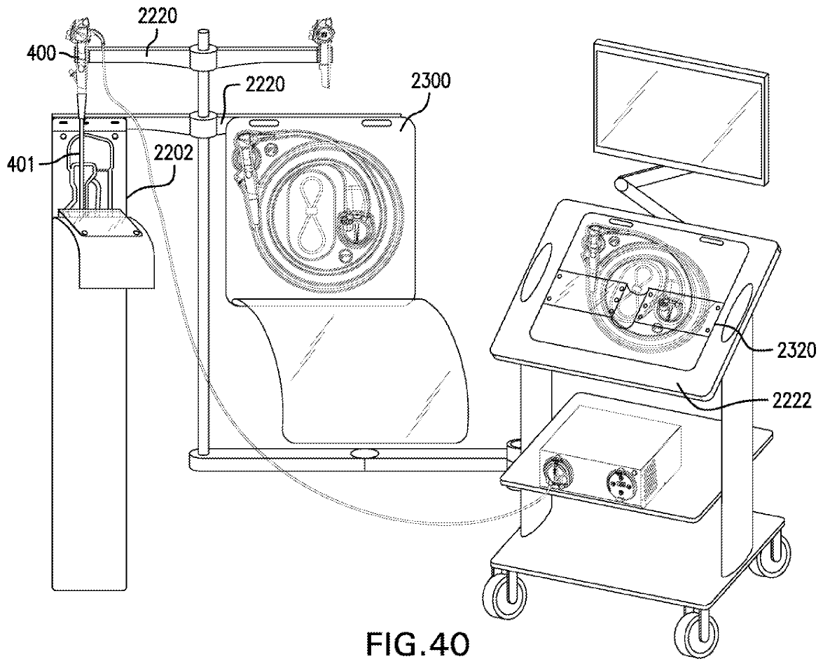

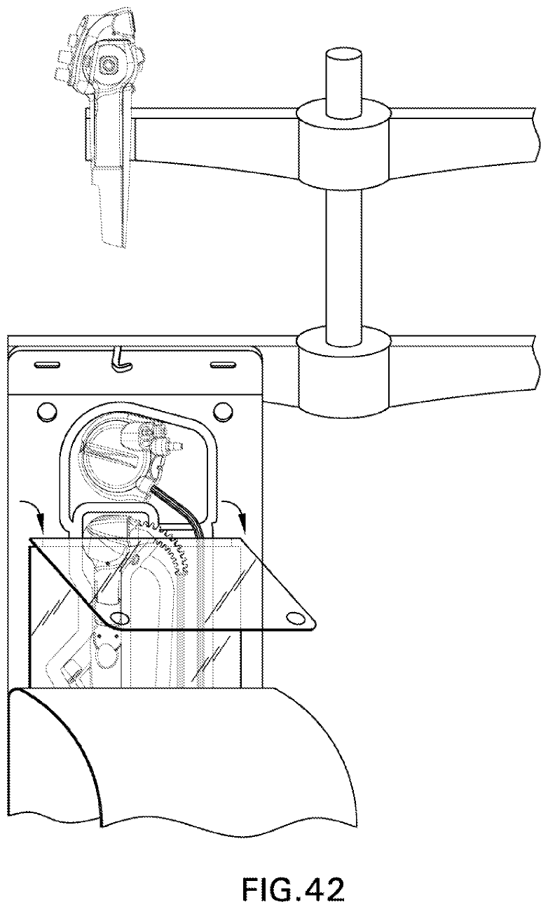

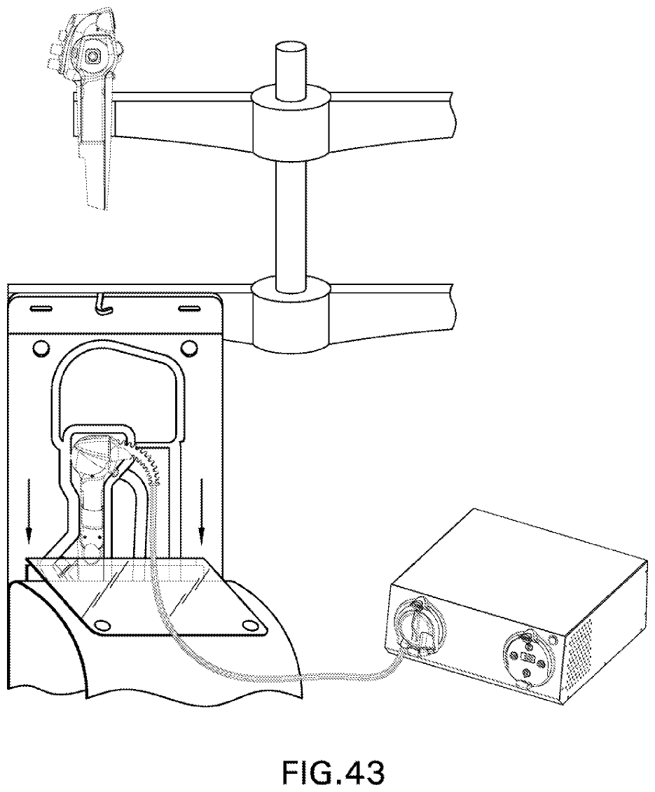

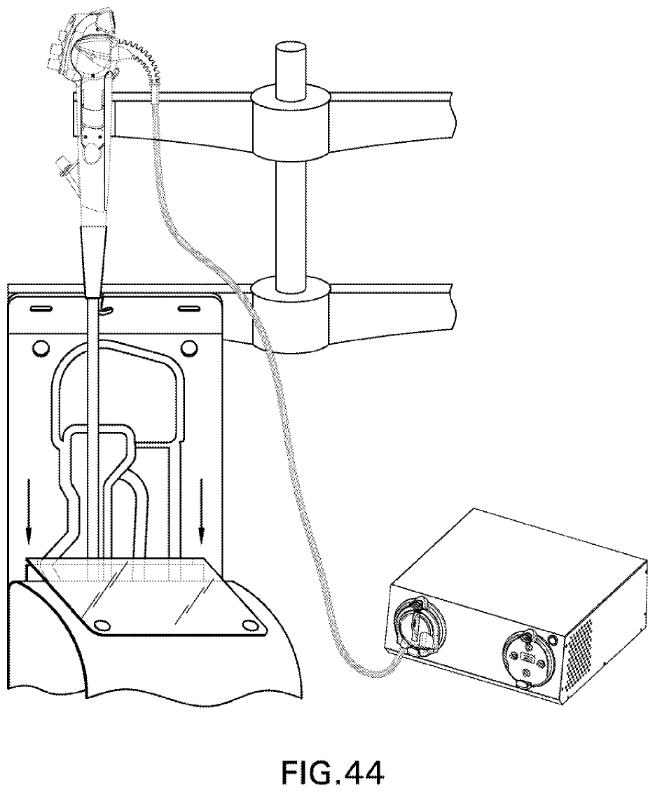

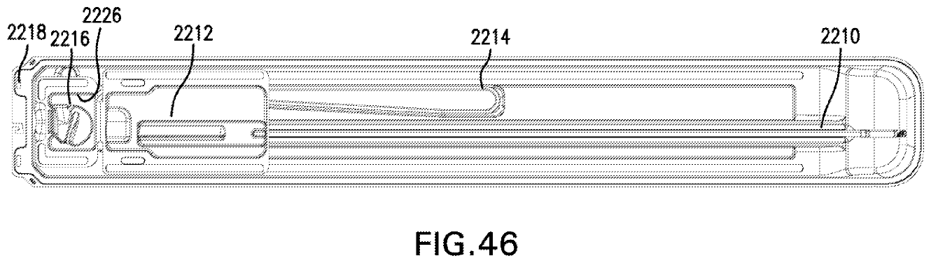

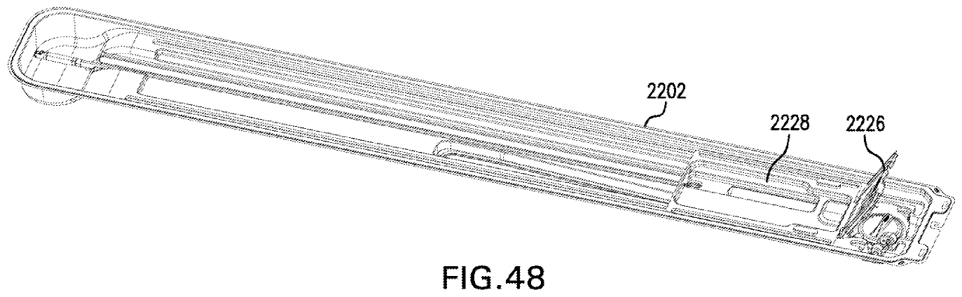

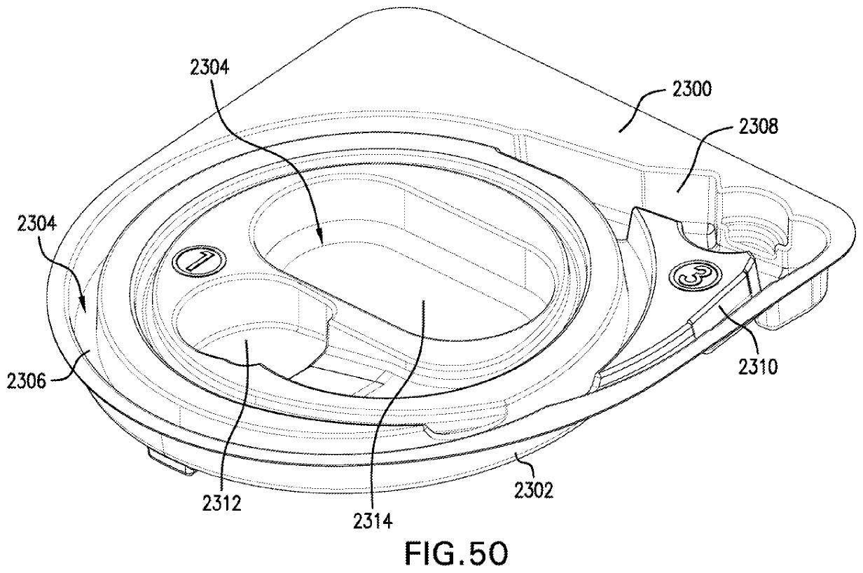

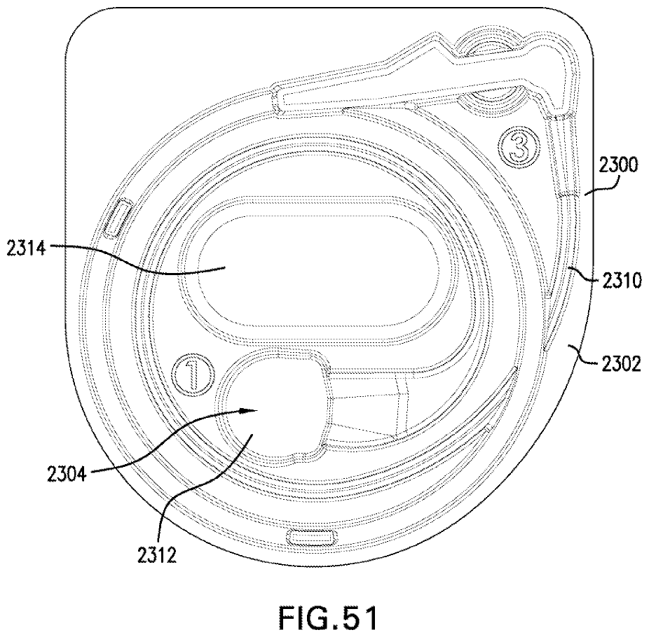

FIGS. 40-52 illustrate a packaging system for and method for maintaining sterility of a medical product, such as an endoscope assembly or a portion of an endoscope assembly. Although it is understood disclosed packaging system can be used for a number of medical products, the disclosure will refer to the medical product as an endoscope assembly for illustrative purposes only. In one example, the packaging assembly 2000 includes an elongated tray 2202 with a first end 2204 and a second end 2206. The tray defines a recess 2208 for retaining the endoscope assembly. In some examples, the tray may be manufactured by thermoforming and may be made of a recyclable material that allows the tray to be recycled after use.

The tray may be formed such that the medical product may only be placed within the tray in one configuration. The tray may be thermoformed from a single piece of material or multiple pieces of material. The tray preferably defines the recess 2208. The recess preferably defines multiple compartments within the recess to house the various components of the endoscope assembly with the tray. The recess may include an insertion tube compartment 2210, single-use shaft assembly compartment 2212, an umbilical compartment 2214 and a cartridge compartment 2216. The recess may include additional compartments if additional components are stored within the tray. The recess may include fewer compartments if fewer components are stored within the same tray. For instance, the cartridge may be sold separately or in a separate container within the packaging system, therefore, there would not be a need for a cartridge compartment within the tray.

The tray may include hanger slots 2218 at the first end of the tray, for example a pair of hanger slots. The hanger slots may be attached as a separate piece or formed out of the material forming the tray. The hanger slots allow the elongated package to be hung from a support arm 2220. The support arm may be attached to the wall or ceiling of the operating room. In some examples, the support arm may be attached to a cart 2222. When attached to the support arm in a vertical orientation a portion or the entirety of the tray is suspended above the floor. Advantageously, such an arrangement may avoid contaminants entering the tray and/or improve ease of use for a medical professional.

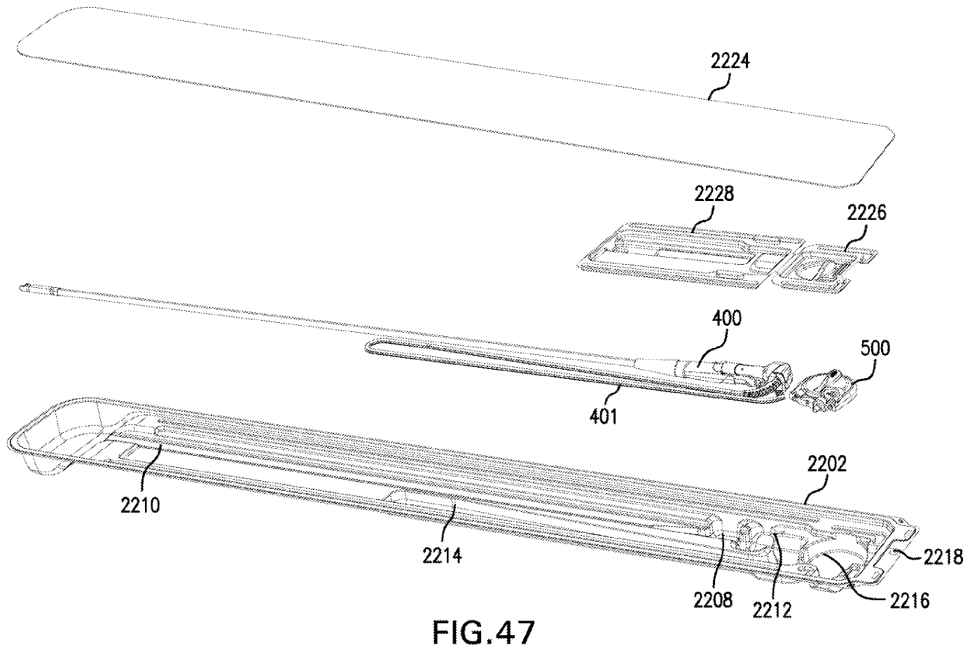

The packaging system may further include a cover 2224 (e.g., a sterile barrier) extending over and sealing the recess portion of the tray from outside contaminants and retaining the endoscope assembly in a sterilely sealed condition (shown in exploded FIG. 47). The cover may be a peel-away cover allowing a user to easily access the endoscope assembly. The cover may be made of a high-density polyethylene fibers, such as Tyvek®.

In some examples, the tray may contain a two piece endoscope including a reusable hand piece and single-use shaft assembly. In the illustrated examples, only the single-use shaft assembly is included in the packaging system. It is understood that a reusable hand piece may be included in the packaging system. The single-use shaft assembly may include an insertion tube at the patient end of the endoscope. The single-use shaft assembly may further include an umbilical extending to and connecting to a cartridge. Any combination of the components within the tray may be provided connected and/or fully assembled with the other components.

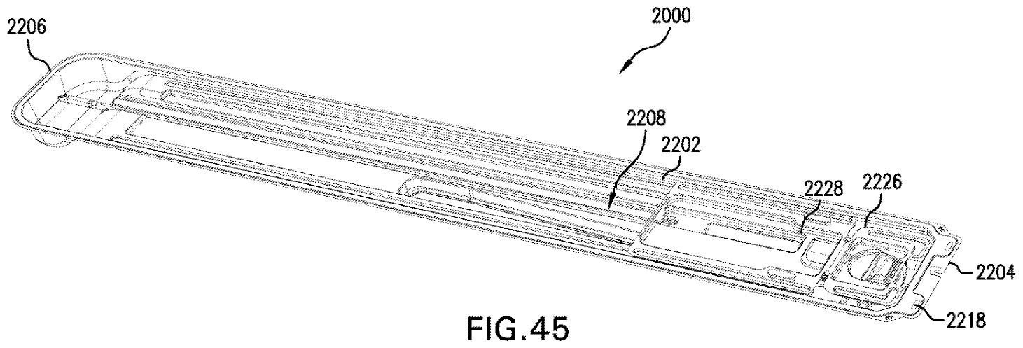

As illustrated in FIGS. 45-49, packaging system may include a first cover 2226, in some examples the packaging system may contain more than one cover. In the illustrated example, a first cover is used to retain the cartridge within the tray (e.g., the cartridge compartment). The packaging system may include a second cover 2228 to retain the single-use shaft assembly within the tray (e.g., the single-use shaft assembly compartment).

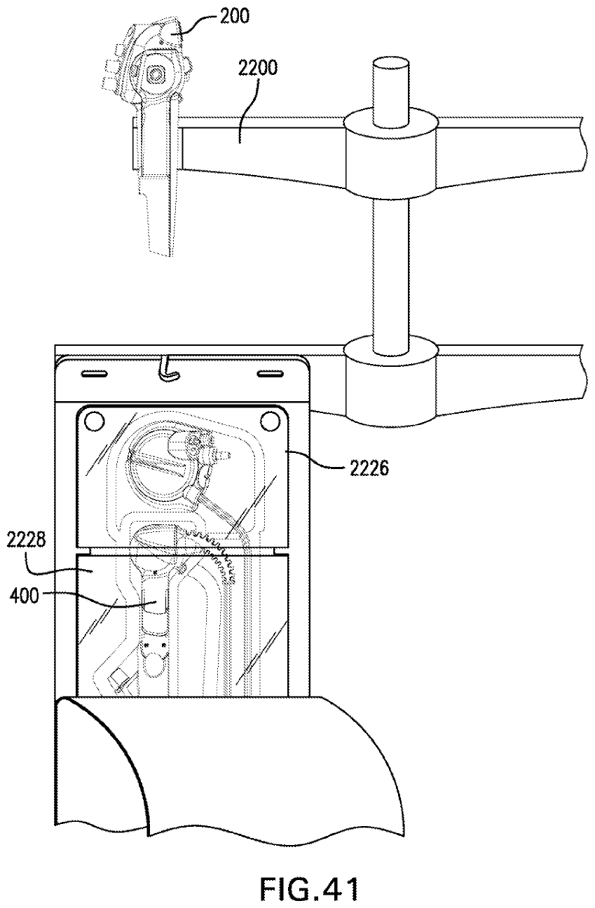

During preparation for a procedure, a user may peel away the cover (e.g. sterile barrier) allowing access to the endoscope assembly. As used herein, user may mean an endoscopist, a nurse, a technician, a doctor or any other individual who may be present prior to or during a procedure. In some examples, during set up, the cover may be peeled away a portion at a time. By only peeling the cover (e.g., sterile barrier) partially, the user may set up the endoscope for procedure while keeping the rest of the endoscope assembly sterile and/or reducing the risk of contamination. In FIGS. 41, 42 and 48, the first cover may be pulled away to reveal the cartridge. The cartridge and umbilical may be removed in order to attach the cartridge to the console (as shown in FIG. 39) prior to or concurrently to preparing the remainder of the endoscope assembly for a procedure.

Advantageously, by peeling away only a portion of the cover (e.g., sterile barrier) and/or fewer than all of the covers, the handle and insertion tube are still protected from various contaminants. Often, as illustrated in FIG. 40, the package system is hung from a support arm. When the package is open the insertion tube may swing during set up/preparation causing the insertion tube to bang against the cart, cart wheels, floor or individuals as they maneuver throughout the room. By keeping the insertion tube covered by the cover (e.g., sterile barrier), although the sterile seal may have been broken, the cover is still protecting the insertion tube from picking up various contaminants by preventing contact with anything within the environment.

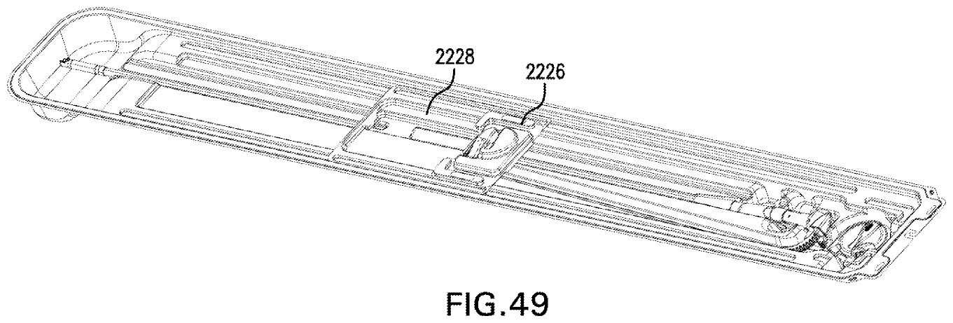

Next, the second cover may be pulled back to access the single-use shaft assembly in the single-use shaft assembly compartment. The second cover (e.g., sterile barrier) may be peeled away further in order to complete this step, depending on whether the user is peeling the cover away a portion at a time or all at once. In some examples, the first cover must be pulled away before the second cover can be pulled away, requiring the user to follow the same set up procedure. In other examples, the second cover is independent of the first cover, allowing both covers to be accessed separately. Once the second cover has been pulled away, the single-use shaft assembly may be removed for set up. To facilitate easy removal of the single-use shaft assembly and the insertion tube, the covers may be configured to slide vertically along the sides of the tray in order to expose more of the insertion tube (as seen in FIG. 49).

As shown in FIGS. 40-44, the reusable hand-piece may be held by a support arm. Once the single-use shaft assembly has been removed from the packaging system, the user may couple the single-use shaft assembly to the reusable hand-piece.

A second example of the packaging system is illustrated in FIGS. 40, 50, 51 and 52. This example is a curved flexible endoscope packaging 2300. The packaging assembly includes tray 2302. The tray defines a recess 2304 for retaining the endoscope assembly. In some examples, the tray may be manufactured by thermoforming and may be made of a recyclable material that allows the tray to be recycled after use.

The tray may be formed such that the medical product may only be placed within the tray in one configuration. The tray may be thermoformed. The tray preferably defines a recess. The recess preferably defines multiple compartments/tracks to house the various components of the endoscope assembly. The recess may include an insertion tube track 2306, single-use shaft assembly compartment 2308, an umbilical track 2310 and a cartridge compartment 2312. The curved packaging system may further include a suction tube compartment 2314 to retain the suction tube. Unlike the elongated tray, the compartments for the insertion tube and umbilical encircle the center of the tray. The umbilical and insertion tube are wound around the center of the packaging. The umbilical and insertion tube tracks may overlap. When the tracks overlap, the umbilical may be positioned on top of the insertion tube. The recess may include additional compartments if additional components are stored within the tray. The recess may include fewer compartments if fewer components are stored within the same tray. For instance, the cartridge may be sold separately or in a separate container within the packaging system, therefore, there would not be a need for a cartridge compartment within the tray.

The tray may include hanger slots 2316 at the top end of the tray, for example a pair of hanger slots. The hanger slots may be attached as a separate piece and/or defined by the material forming the tray. The hanger slots allow the elongated package to be hung from a support arm 2220. The support arm may be attached to the wall or ceiling of the operating room. In some examples, the support arm may be attached to a cart. When attached to the support arm the entirety of the tray is suspended above the floor. Due to the compact configuration of the curved packaging system, the tray may be placed on a cart rather than hung from a support arm if desired.

The packaging system may further include a cover (e.g., sterile barrier) extending over and/or sealing the recess portion of the tray from outside contaminants and retaining the endoscope assembly in a sterilely sealed condition. The cover may be a peel-away cover allowing a user to easily access the endoscope assembly. The cover may be made of a high-density polyethylene fibers, such as Tyvek®.

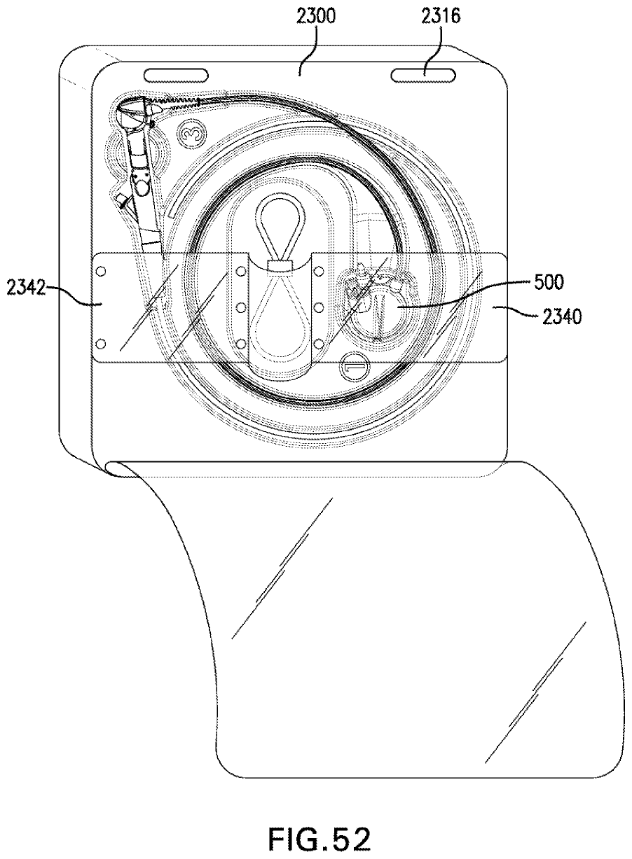

Similar to the elongated packaging system, the curved packaging system may include a first cover 2340 (illustrated in FIG. 52), in some examples the packaging system may contain more than one cover. In the illustrated example, the first cover is used to retain the cartridge within the tray/cartridge compartment. The packaging system may include a second cover 2342 to retain the single-use shaft assembly within the tray/single-use shaft assembly compartment.

Similar to the elongated packaging system, the curved packaging system may be designed to encourage the user to remove the endoscope assembly in a desired sequence. During set up, the cover (e.g., sterile barrier) may be partially and/or completely removed (e.g., peeled away) to access the entirety of the endoscope assembly. Due to the configuration of the packaging system, the dangers of contamination present in the elongated packaging system are reduced.

First the first cover retaining the cartridge may be moved to access the cartridge. The cartridge and umbilical may be removed and the cartridge may be attached to the console. Next, the second cover may be moved to access the single-use shaft assembly. The single-use shaft assembly may be removed from the tray and attached to the reusable hand piece assembly. Finally, the insertion tube may be removed and inserted into the patient or placed on a sterile surface until the procedure commences.

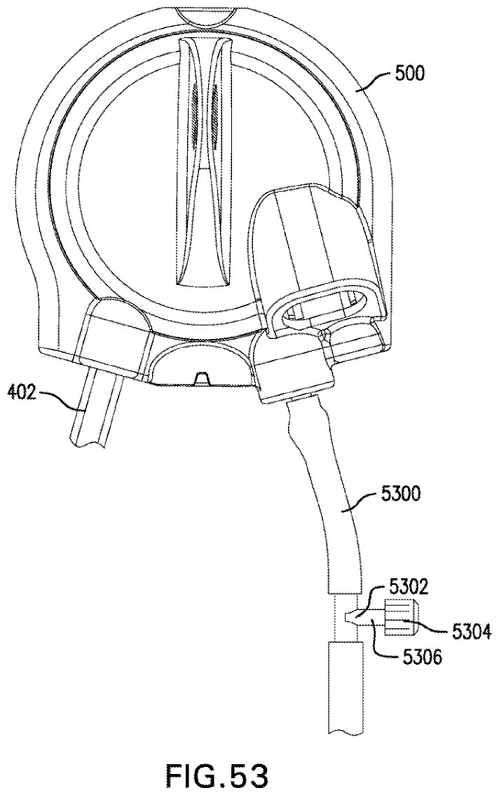

FIG. 53 illustrates a cartridge 500 with a suction tube 5300 extension incorporating a connector, for example a t-fitting 5302. This configuration may be used with a flexible single-use viewing endoscope that incorporates a suction tube with an inline t-fitting. The t-fitting may include a removable cap 5304. The removable cap protecting a side port 5306 of the t-fitting from admitting contaminants when the t-fitting is not in use and removable when use is desired. The side port of the t-fitting may mate with a connector on a cholangioscope suction tube.

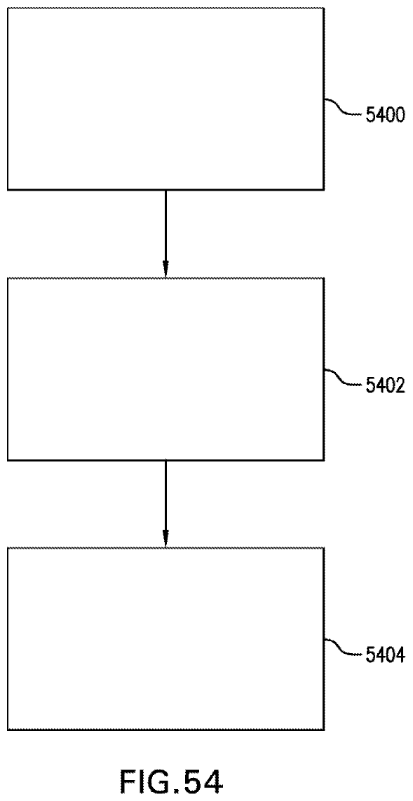

FIG. 54 illustrates a method of selecting between the imaging devices of one or more endoscopes to display on a monitor during an endoscopic procedure. Often, during endoscopic procedures, multiple endoscopes are utilized to perform the procedure. This method is directed toward identifying which endoscope's image to display on the monitor. In some embodiments, multiple monitors may be used. In other embodiments, a single monitor may be used to display all of the endoscopes' images on one monitor. When using one monitor, the endoscope being used may be identified and displayed as the most dominate or largest image being displayed among other smaller images on the same screen/monitor.

The method may include identifying the endoscope being used at a certain point during a procedure. 5400 The endoscope may be identified using a switch, for example a mechanical, inductive or capacitive switch (e.g., a touch switch). The switch may be a sensor. In some embodiments, the switch is a touch switch, so that when an endoscope is picked up by an endoscopist, the switch is activated so as to identify the endoscope currently being used in the procedure. When the switch is activated, a signal is relayed from the switch to a controller, such as to identify which endoscope is being used 5402. The controller relays the signal/image from the associated endoscope to a monitor 5404. The identified image being the dominate image on the screen while the endoscope is still in use. In some examples, multiple endoscopes may be used simultaneously. When multiple endoscopes are used simultaneously, the images from the endoscopes in use may be displayed on the monitor as the same size. In other examples, a dominate endoscope may be identified. When the dominate endoscope is being used simultaneously with another endoscope, the image relayed from the dominate endoscope may be the largest image on the monitor.

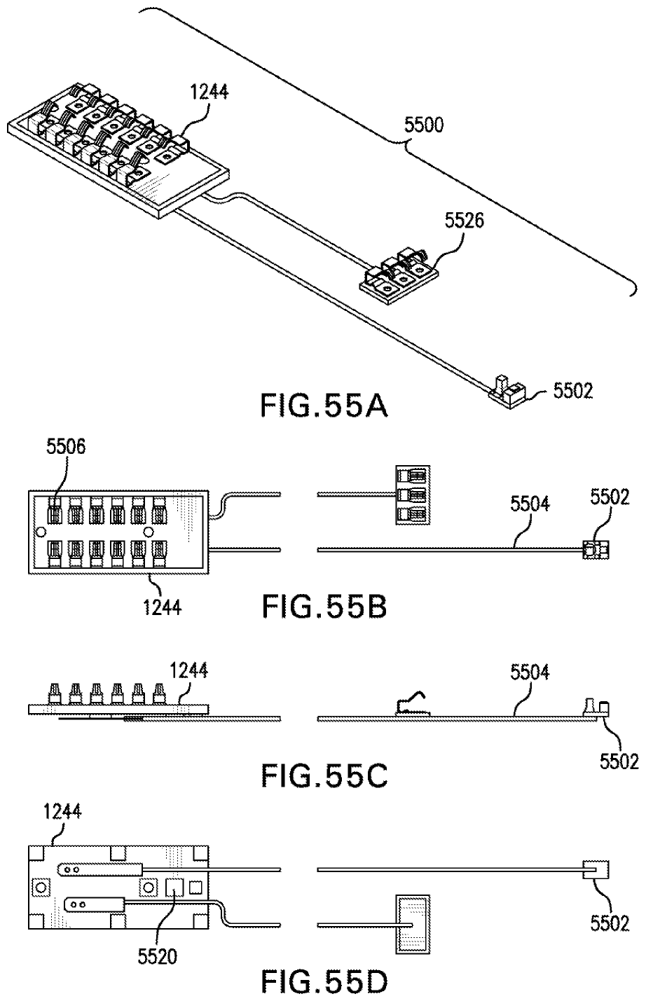

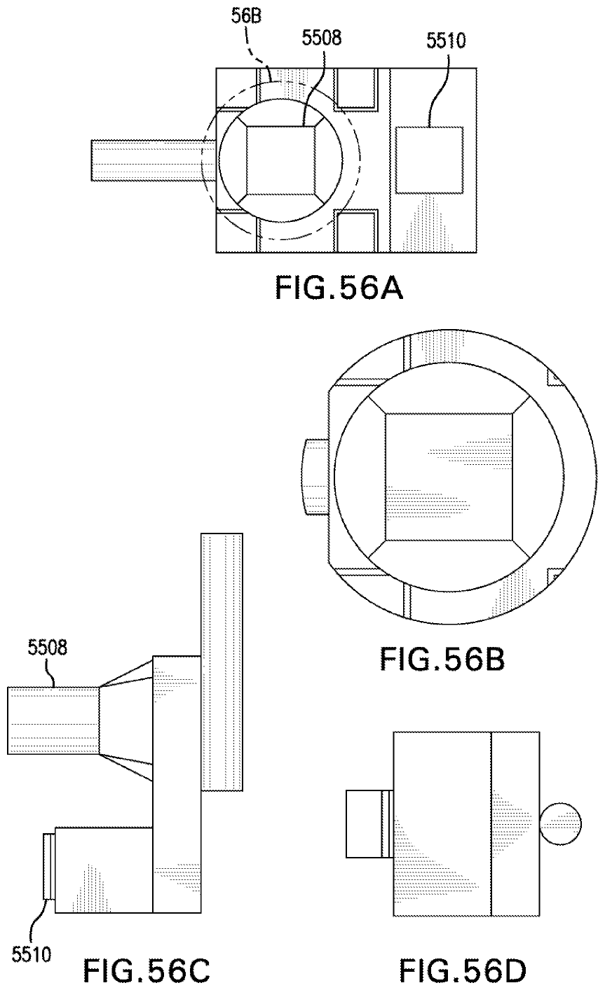

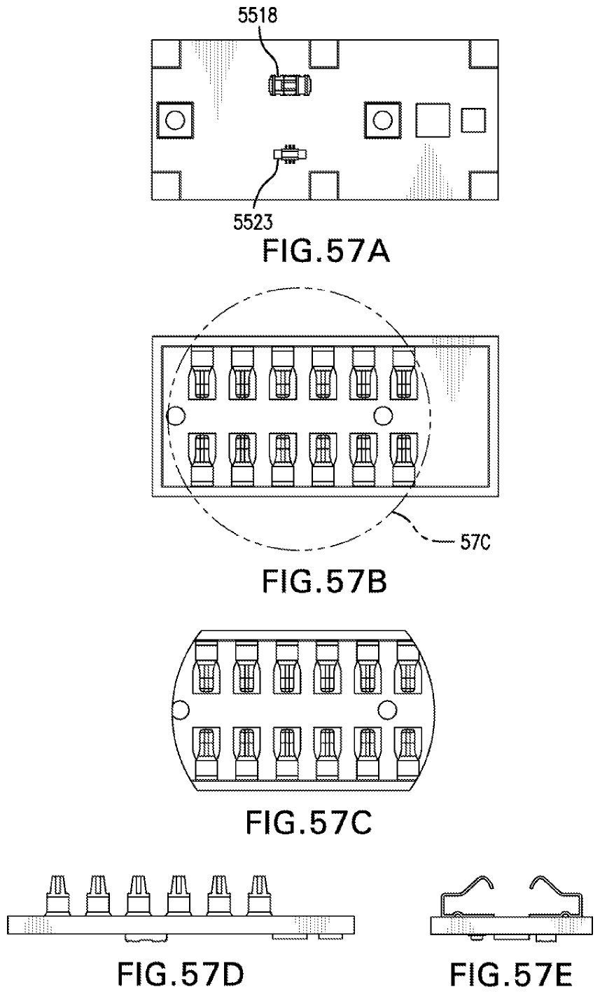

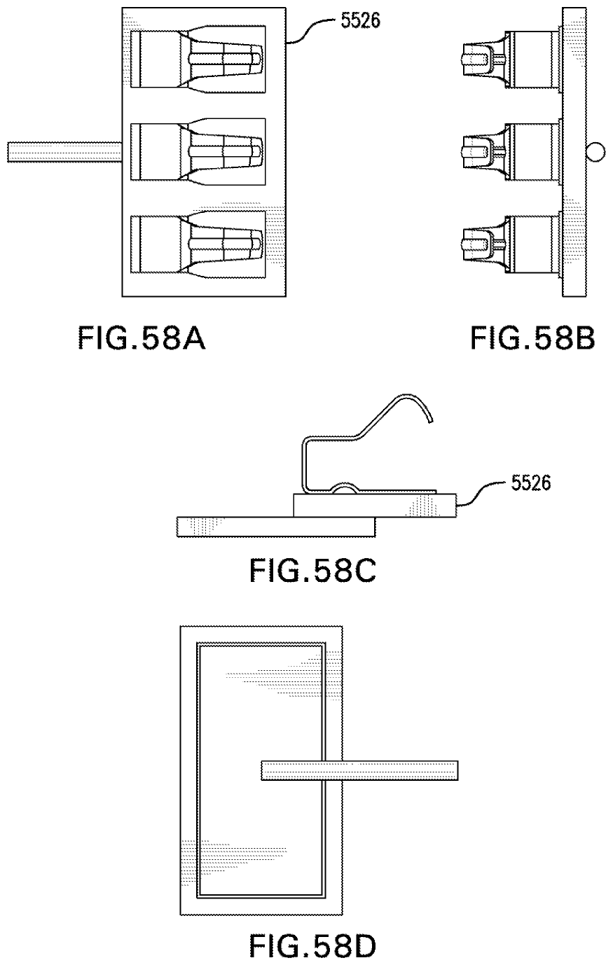

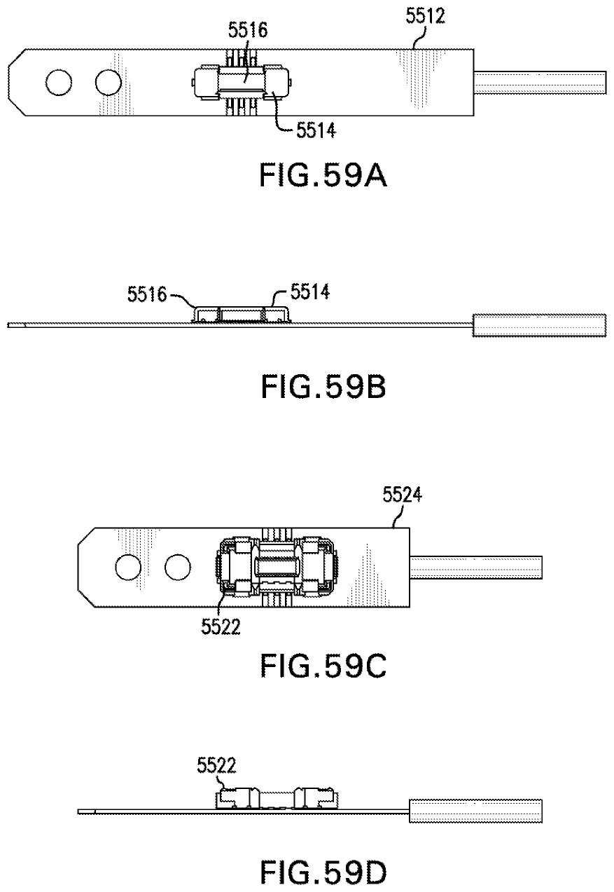

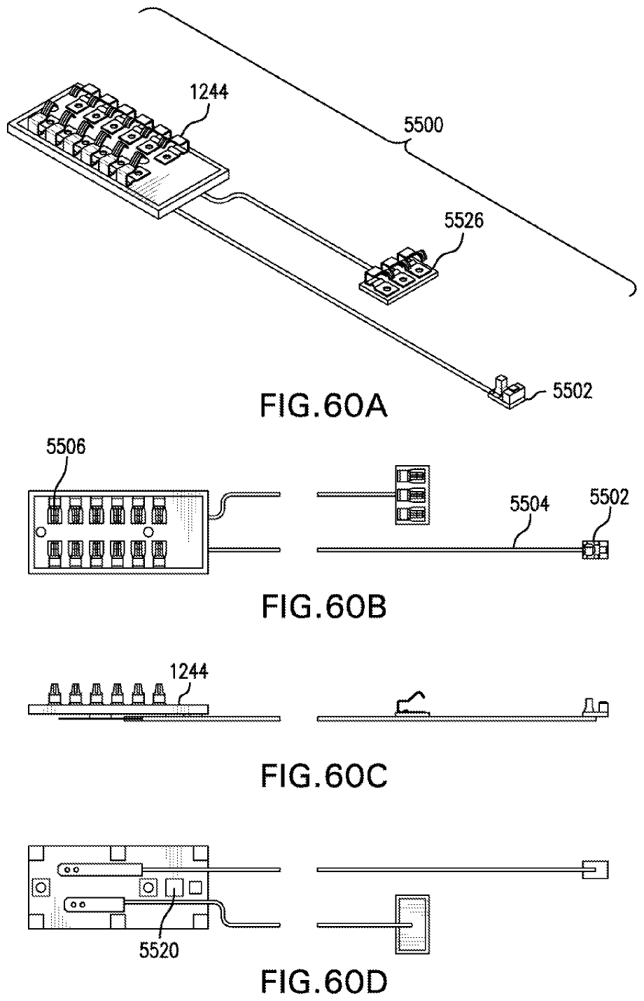

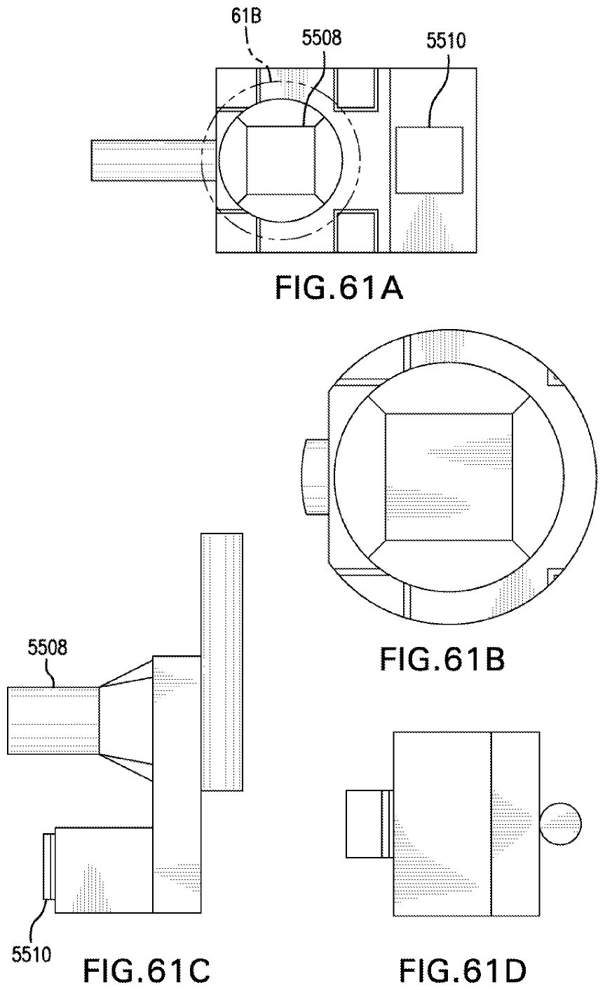

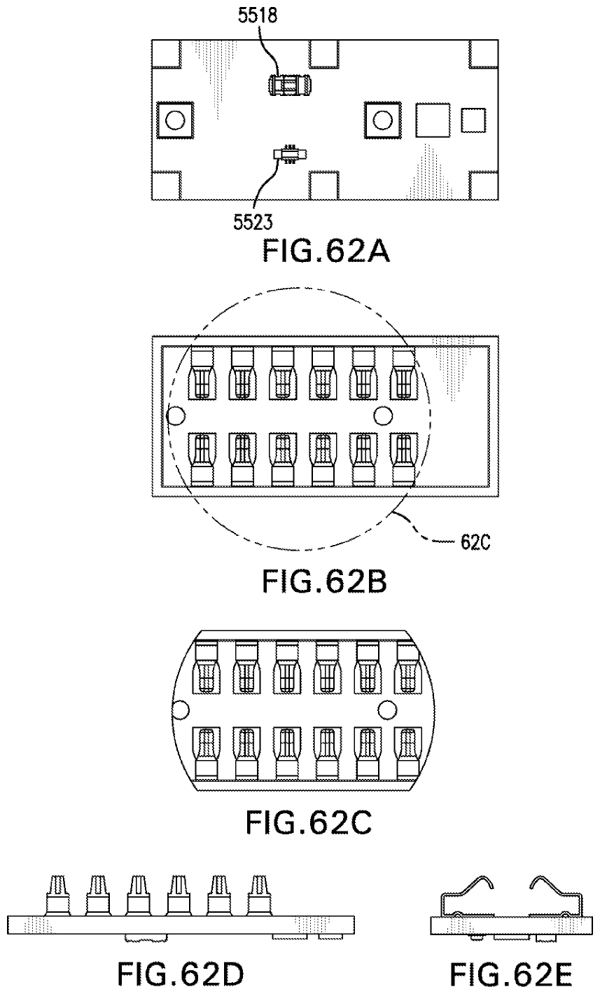

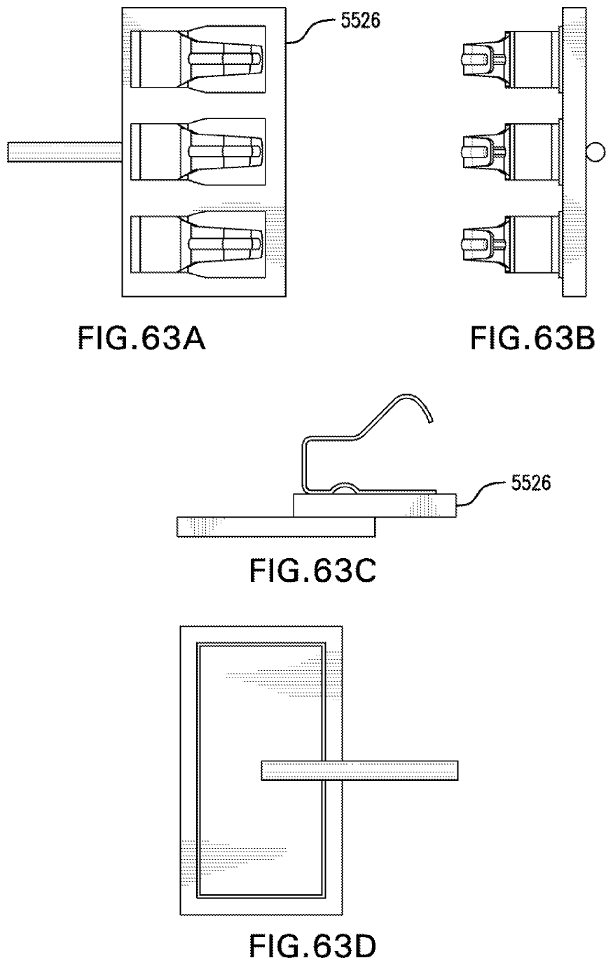

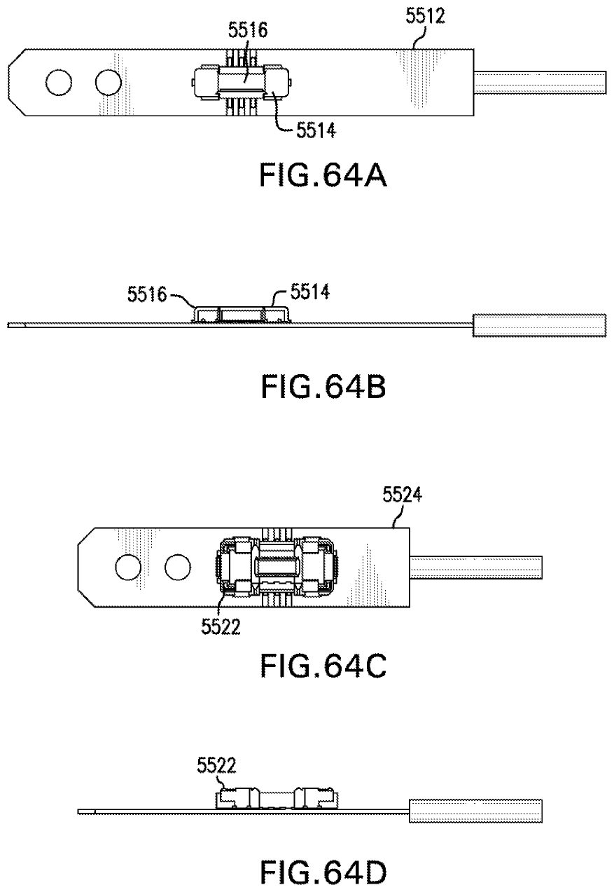

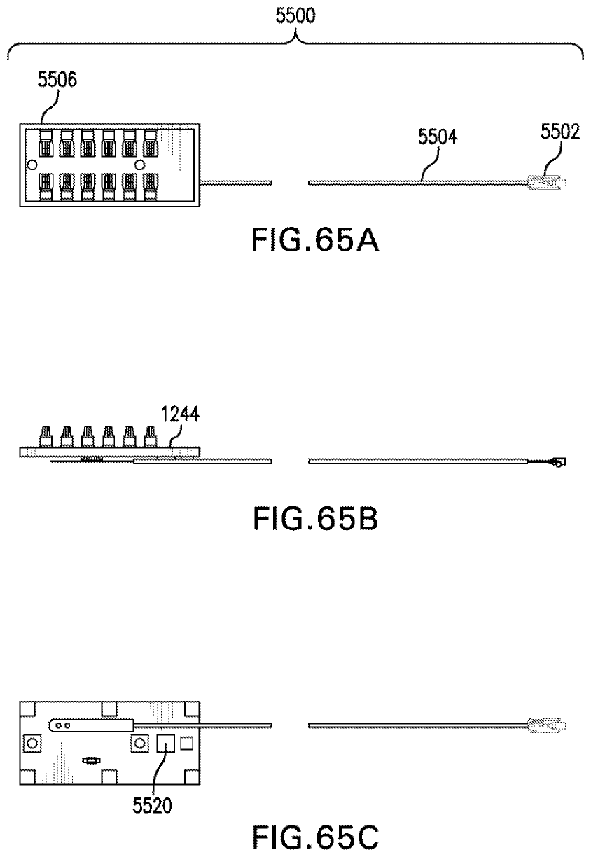

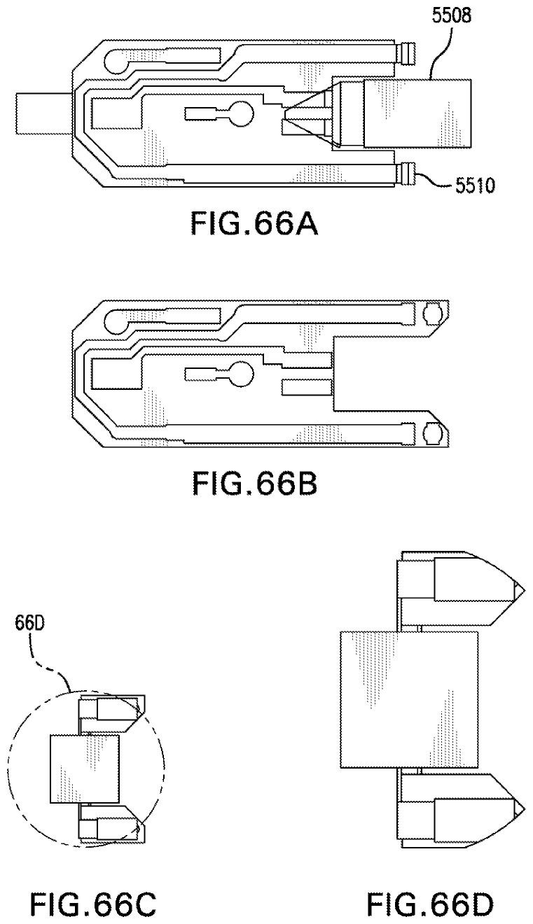

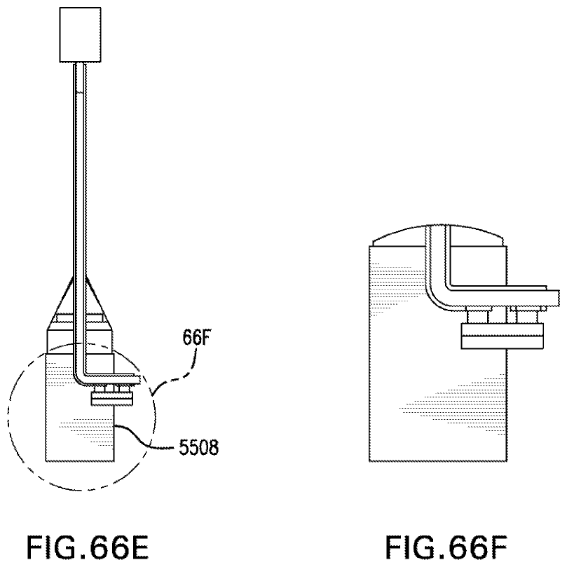

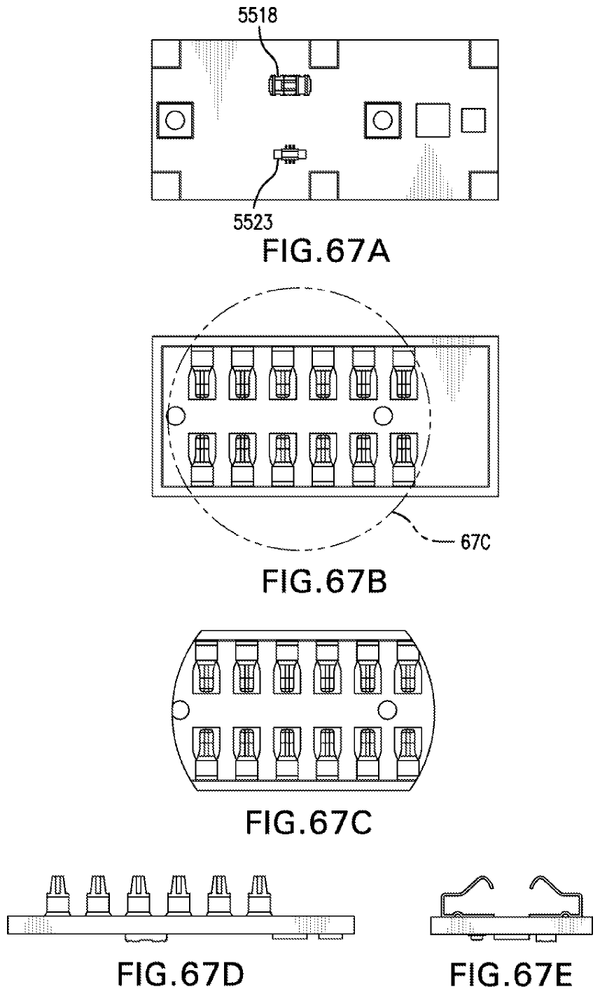

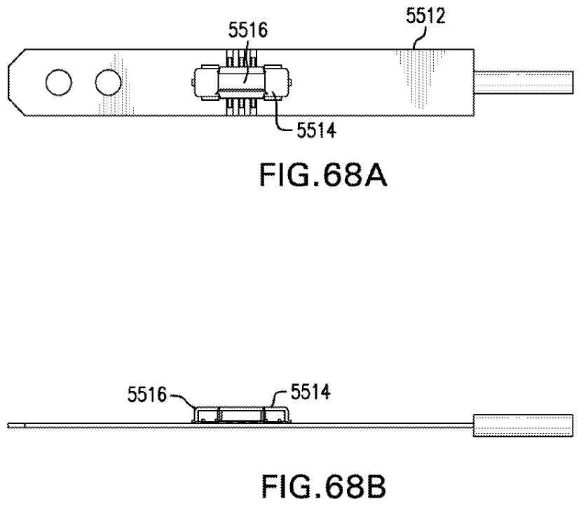

FIGS. 55-68 illustrates three embodiments of image sensor assemblies 5500 used in various types of endoscopes, for example gastroscopes, colonoscopes, duodenoscopes or cholangioscopes. Broadly, a camera and LED board 5502 may be arranged at the distal end of the insertion tube and a cable 5504 with electrical connectors may extend from the distal end of the insertion tube to the proximal end of the insertion tube and to the electrical connector/circuit board 1244 in the cartridge. As described above, the electrical connector of the cartridge interfaces with the console and passes electrical signals between the cartridge and the console. In some embodiments, the electrical connector of the cartridge may include spring finger electrical connectors 5506 to contact with spring fingers on the console.

The image sensor assembly includes a camera and LED board. A side of the camera and LED board is bonded with a cable. The cable may be bonded to the board with any method know in the art, for example with an adhesive. Extending from the distal end of the cable and soldered to the board are a plurality of wires (not shown). The wires are soldered to the board to carry signals between the cartridge and a camera 5508 and a LED 5510. The other ends of the same wires extend from the proximal portion of the cable and are soldered on the surface of a flex circuit 5512. The wires may be appropriately spaced apart across the surface of the flex circuit.

The flex circuit may be operably attached to a micro connector 5514. The micro connector may be either a male or a female portion 5516 with the opposite portion 5518 located on the electrical connector of the cartridge. The male and female portions are connected to complete the electrical path between the camera and/or LED and the cartridge. In some embodiments, the micro connector may be a mezzanine connector.

The flex circuit and micro connector preferably have a combined height of less than 1 mm. More preferably, the combined height of the flex circuit and micro connector is less than 0.8 mm. Such arrangements may have a combined height in the range of 0.6 mm to 0.8 mm. The flex circuit itself may have a height of about 0.2 mm.

During assembly, the proximal end of the wire including the flex circuit and micro connector are passed into the distal end of the insertion tube and through the length of the insertion tube towards the proximal end of the insertion tube. The flex circuit and micro connector may then be passed through the endoscope and umbilical to the cartridge operably connected at the end of the umbilical. The female or male portion of the micro connector located on the flex circuit is then connected with the corresponding portion of the micro connector located on the electrical connector of the cartridge. Advantageously, this method of manufacturing avoids passing the cable through the insertion tube before connecting the camera and LED board to the cable, which can be challenging and time consuming. Moreover, the method avoids passing the camera through the insertion tube, which may damage the camera and/or require a larger insertion tube to accommodate the camera.

The circuit board may further include a storage device 5520 to store the imaging data from the camera.

The image sensor assembly may further include a second micro connector 5522 on the cartridge electrical connector connectable to a second connector 5523 of the electrical connector/circuit board 1244 in the cartridge. The second micro connector is also attached to a flex circuit 5524 similarly to the arrangement in the description above. The flex circuit includes a wire extending from the flex circuit on the cartridge circuit board to a single-use shaft assembly electrical connector 5526. The single-use shaft assembly electrical connector is connectable to the electric connector of the reusable hand-piece to pass electrical signals/commands between the two endoscope pieces.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, “left”, “right”, and the like, are used herein solely for the convenience of the reader in order to aid in the reader's understanding of the illustrated examples, and it is not the intent that the use of these directional terms in any manner limit the described, illustrated, and/or claimed features to a specific direction and/or orientation.

The language used in the claims and the written description is to only have its plain and ordinary meaning, except for terms explicitly defined below. Such plain and ordinary meaning is defined here as inclusive of all consistent dictionary definitions from the most recently published (on the filing date of this document) general purpose Merriam-Webster dictionary.

As used in the claims and the specification, the following terms have the following to defined meanings:

The term “friction fit” as used herein means held together by friction after the parts are pushed together. The term does not refer to securing with fasteners (e.g., screws, bolts) or adhesives.

While the disclosure has been illustrated and described in detail in the drawings and foregoing description, the same is to be considered as illustrative and not restrictive in character, it being understood that only the preferred example has been shown and described and that all changes, equivalents, and modifications that come within the spirit of the disclosures defined by following claims are desired to be protected. All publications, patents, and patent applications cited in this specification are herein incorporated by reference as if each individual publication, patent, or patent application were specifically and individually indicated to be incorporated by reference and set forth in its entirety herein.

The following numbered clauses set out specific examples that may be useful in understanding the present disclosure:

1. A packaging system for a medical product having multiple components, comprising:
a tray defining a recess for retaining the multiple components of the medical product in a sterile environment;
a first cover coupled to the tray, wherein the first cover retains a first component of the multiple components within a first portion of the recess;
a second cover coupled to the tray, wherein the second cover is not removable from the tray until the first cover is at least partially removed, and wherein the second cover retains a second component of the multiple components within a second portion of the recess; and a third cover covering the first cover and the second cover and providing a sterile barrier over the recess of the tray.
   2. The packaging system of clause 1, further comprising:
a medical product box, wherein the tray and the first, second, and third cover are disposed within said medical product box.
   3. The packaging system of any preceding clause, further comprising:
a slot at a first end of the tray and configured to receive a support structure for hanging the tray in a vertical orientation.
   4. The packaging system of any preceding clause, wherein the first cover retains the first component of the medical product within a first portion of the recess when the tray is hanging vertically.
   5. The packaging system of any preceding clause, wherein the second component is a single-use shaft assembly that is selectively attachable to a reusable hand-piece of an endoscope system.
   6. The packaging system of any preceding clause, wherein the first component is a cartridge attachable to a pump.
   7. The packaging system of any preceding clause, further comprising:
a fourth cover coupled to the tray, wherein the fourth cover retains a suction tube within the recess of the tray.
   8. The packaging system of clause 7, wherein the fourth cover is positioned under the third cover.
   9. A method, comprising:
removing at least a portion of a first cover of a medical package to expose a first portion of a recess of the medical package and expose a second cover of the medical package;
removing from the first portion a cartridge configured to attach to a fluid pump;
removing at least a portion of the second cover of the medical package to expose a second portion of the recess of the medical package after removing the first cover; and
removing from the second portion of the recess a handle portion of a single-use shaft assembly of an endoscope.
   10. The method of clause 9, further comprising removing an insertion portion of the single-use shaft assembly from the second portion of the recess after removing said handle portion.
   11. The method of clause 10, wherein said removing the insertion portion of the medical package includes removing the first cover to expose the insertion portion.
   12. The method of any one of clauses 9-11, further comprising removing a third cover forming a sterile barrier over the first cover and the second cover.
   13. The method of clause 12, wherein:
said removing the third cover occurs prior to removing said first and second covers.
   14. A method of packaging a medical device, comprising:
positioning a second component of the medical device within a second portion of a recess defined by a tray;
covering the second component with a second cover;
positioning a first component of the medical device within a first portion of the recess defined by the tray;
covering the first component and at least part of the second cover with a first cover; and
covering at least the first cover and the second cover with a third cover to define a sterile environment within the recess.
   15. The method of packaging the medical device of clause 14, further comprising:
positioning a third component of the medical device within a third portion of the recess.
   16. An endoscope, comprising:
an insertion tube having a distal end, wherein the distal end of the insertion tube includes a distal tip;
an opening in a surface of the distal tip, wherein an instrument inserted through an instrument tube extending within the insertion tube extends out of the opening; and
an elevator assembly, including:
   an elevator with a proximal end and a distal end;
   a distal cap over at least a portion of the elevator assembly; and
   a pair of opposing notches to limit movement of the instrument extending out of the opening;
   wherein a first notch of the pair of opposing notches is defined by the distal end of the elevator; and
   wherein a second notch of the pair of opposing notches is defined by the distal cap.
      17. The apparatus of clause 16, wherein the elevator assembly further includes:
an elevator control wire operably coupled to the elevator, wherein a force exerted on the elevator control wire adjusts the height of the elevator.
   18. The apparatus of clause 17, wherein the elevator control wire is a single strand pre-formed wire.
   19. The apparatus of clause 16 or clause 17, wherein the elevator control wire translates over a static curved ramp resulting in a rotational torque on the elevator.
   20. The apparatus of any one of clauses 16-19, wherein the distal cap is removable from the elevator assembly.
   21. A medical device, comprising:
a reusable hand-piece assembly and a single-use shaft assembly;
the reusable hand-piece assembly selectively attachable to and detachable from the single-use shaft assembly;
the single-use shaft assembly including a housing and an insertion tube; and
wherein the housing contains an elevator control assembly for controlling the operation of an elevator at the distal end of the insertion tube;
wherein the elevator control assembly has an actuatable lever extending out of the housing, wherein said lever is operably coupled to actuate an elevator driving rack, wherein said elevator driving rack is operably coupled to actuate a pinion, wherein said pinion is operably coupled to actuate an elevator driven rack, wherein said elevator driven rack is operably coupled to actuate an elevator control wire, and wherein a force exerted on the elevator control wire adjusts the height of the elevator.
   22. A medical device, comprising:
a first pulley associated with a first articulation wire of the medical device for deflection of a distal end of the medical device in a first plane upon rotation of the first pulley;
wherein the first pulley is rotatable no more than a first rotational distance relative to a rotational stop idler; and
wherein the rotational stop idler is rotatable no more than a second rotational distance relative to a housing of the endoscope; and
wherein the first pulley is rotatable relative to the housing no more than the sum of the first rotational distance and the second rotational distance.
   23. The medical device of clause 22, wherein the rotational stop idler has a first side contacting the first pulley and a second side contact the housing.
   24. The medical device of clause 23, wherein the first side includes a first groove and the second side includes a second groove;
wherein a boss on the first pulley is slidably received within the first groove of the rotational stop idler; and
wherein a boss on the housing is slidably received within the second groove of the rotational stop idler.
   25. The medical device of any one of clauses 22-24, comprising:
a second pulley associated with a second articulation wire of the medical device for deflection of the distal end of the medical device in a second plane upon rotation of the second pulley;
wherein the second pulley is rotatable no more than a third rotational distance relative to a second rotational stop idler;
wherein the second rotational stop idler is rotatable no more than a fourth rotational distance relative to the housing of the endoscope; and
wherein the second pulley is rotatable relative to the housing no more than the sum of the third rotational distance and the fourth rotational distance.
   26. The medical device of clause 25, wherein the second plane is orthogonal to the first plane.
   27. The medical device of clause 25 or clause 26, wherein the second rotational stop idler has a first side contacting the second pulley and a second side contacting the housing.
   28. The medical device of clause 27, wherein the first side of the second rotational stop idler includes a first groove and the second side includes a second groove;
wherein a boss on the second pulley is slidably received within the first groove of the second rotational stop idler; and
wherein a boss on the housing is slidably received within the second groove of the second rotational stop idler.
   29. The medical device of any one of clauses 22-28, wherein the housing is part of a single-use shaft assembly; and
wherein the single-use shaft assembly is selectively attachable and detachable to a reusable hand-piece assembly having a control knob and shaft for rotating the first pulley.
   30. The medical device of any one of clauses 22-30, wherein the medical device is an endoscope.
   31. A medical device, comprising:
a reusable hand-piece assembly and a single-use shaft assembly;
the reusable hand-piece assembly selectively attachable to and detachable from the single-use shaft assembly; and
the reusable hand-piece assembly including a housing and an articulation and brake assembly, wherein the articulation and brake assembly includes a first articulation control knob, a second articulation control knob, and a first articulation brake knob; and
wherein the first articulation control knob, the second articulation control knob, and/or the first articulation brake knob are selectively attachable and detachable from the reusable hand-piece assembly.
   32. The medical device of clause 31, wherein the first articulation control knob, the second articulation control knob, and/or the first articulation brake knob are secured to the reusable hand-piece assembly only by friction fit.
   33. The medical device of clause 31 or clause 32, wherein the articulation and brake assembly is secured to the housing of the reusable hand-piece assembly by a retention clip.
   34. The medical device of clause 33, wherein the retention clip is coupled to an up-down articulation hub to secure the articulation knob and brake assembly to the housing of the reusable hand-piece assembly.
   35. An endoscope, comprising:
a first articulation brake configurable from a disengaged position that permits rotation of a first articulation control knob relative to a housing of the endoscope to an engaged position that resists rotation of the first articulation control knob relative to the housing;
wherein rotation of the first articulation control knob relative to the housing rotates a first articulation control shaft around a first axis;
wherein the articulation brake includes braking surfaces at a non-orthogonal angle relative to the first axis;
wherein when the first articulation brake is configured in the engaged configuration the braking surfaces are forced towards one another with a normal force having a first magnitude; and
wherein when the first articulation brake is configured in the disengaged position the braking surface are either separated or contacting one another with a normal force having a magnitude less than said first magnitude.
   36. The endoscope of clause 35, wherein the housing is part of a reusable hand-piece assembly selectively attachable to and detachable from a single-use shaft assembly of the endoscope.
   37. A medical device, comprising:
a pulley;
an articulation wire coupled with the pulley and extending along a direction away therefrom; and
a pulley housing enclosing the pulley and the articulating wire, the pulley housing comprising a first housing portion and a second housing portion;
wherein the first housing portion has a first edge and the second housing portion has a second edge mating with said first edge and defining a non-linear path extending along the direction of the articulation wire.
   38. The medical device of clause 37, wherein the non-linear path is non-linear as projected onto a plane including the articulation wire and parallel to a rotational axis of the pulley.
   39. The medical device of clause 37, wherein the non-linear path is rectilinear.
   40. The medical device of clause 39, wherein the non-linear path defines a castellation.
   41. An endoscope, comprising:
an insertion tube; and
an instrument tube positioned within the insertion tube;
wherein the instrument tube comprises a first portion having a first composition comprising High Density Poly Ethylene and a second portion having a second composition comprising material selected from the group consisting of: polyvinyl chloride or polylactic acid;
wherein the first composition defines an inner surface of the instrument tube defining a tool channel; and
wherein the second composition defines an outer surface of the instrument tube.
   42. The endoscope of clause 41, wherein the instrument tube is formed by coextrusion of the first composition and the second composition.
   43. An endoscope, comprising:
an insertion tube for inserting into a patient's body;
an articulating joint assembly within the insertion tube, the articulation joint assembly having a length extending from a proximal end to a distal end;
wherein the articulating joint assembly comprises a plurality of articulation links;
wherein each articulation link is connected at a contact point to an adjacent link and pivotable a pivot angle relative to the adjacent link;
wherein the pivot angle between a first pair of articulation links of the articulating joint assembly is not the same as the pivot angle between a second pair of articulation links of the articulating joint assembly.
   44. The endoscope of clause 43, wherein the pivot angle between a pair of articulation links proximate the distal end of the articulation joint assembly is greater than the angle between a pair of articulation links located proximally of the pair of articulation links proximate the distal end.
   45. The endoscope of clause 43, wherein the pivot angle between a pair of articulation links proximate the distal end of the articulation joint assembly is less than the angle between a pair of articulation links located proximally of the pair of articulation links proximate the distal end.
   46. The endoscope of clause 43, wherein the pivot angles continually increase along a length of at least four articulation links.
   47. A console, comprising:
a control valve assembly configured to receive a cartridge;
said control valve assembly including a plurality of actuators for actuating fluid paths of the cartridge;
wherein the cartridge includes a magnet;
wherein the control valve assembly includes a magnetic sensor arranged to sense the magnet on the cartridge when the cartridge is connected to the console; and
wherein extension and/or retraction of the plurality of actuators of the console is determined based on a signal from the magnetic sensor.
   48. A console, comprising:
a control valve assembly configured to receive a cartridge;
said control valve assembly including a plurality of actuators for actuating fluid paths of the cartridge;
said plurality of actuators movable from a retracted position to an extended position;
wherein the cartridge includes at least two magnets;
wherein the control valve assembly includes a first magnetic sensor and a second magnetic sensor, the first and second magnetic sensors arranged to sense the at least two magnets on the cartridge when the cartridge is connected to the console;
wherein actuator valves of the console are retracted when the presence of fewer that the at least two magnets are sensed by the first and second magnetic sensors; and
wherein actuator valves of the console are extended when the at least two magnets are sensed by the first and second magnetic sensors.
   49. A cartridge, comprising:
a housing and first tubing extending through the housing, the first tubing defining a first fluid path; and
a connector incorporated inline with the first tubing;
wherein the connector connects with second tubing defining a second fluid path so that the second fluid path merges with the first fluid path.
   50. The cartridge of clause 49, wherein the connector is a t-fitting.
   51. The cartridge of clause 49, wherein the connector includes a removable cap.
   52. A method, comprising:
detecting manipulation of a first medical device by a medical professional during a procedure using first and second medical devices, wherein manipulation of the first medical device is detected by a first switch;
identifying the first medical device as being manipulated from the signal from the first switch; and
configuring an image displaying device to display an image generated from the first medical device.
   53. The method of clause 52, wherein the first switch is a mechanical switch.
   54. The method of clause 53, wherein the first switch is a touch switch.
   55. The method of clause 52, wherein the first switch is activated when the medical device is picked up.
   56. The method of clause 52, wherein the image generated is the image from a camera a distal end of the identified medical device.
   57. The method of clause 52, comprising detecting manipulation of the second medical device by a medical professional during the procedure, wherein manipulation of the second medical device is detected by a second switch;
identifying the second medical device as being manipulated from the signal from the second switch; and
configuring the image displaying device to display an image generated from the second medical device.
   58. The method of clause 57, wherein the image displaying device displays images from both the first medical device and the second medical device simultaneously, but the image from the medical device identified as being used is larger on the image displaying device.
   59. The method of clause 52, wherein only one image from the first or second medical device is displayed on the image displaying device at a time.
   60. An insertion tube assembly of an endoscope, comprising:
an insertion tube;
an electrical cable with a plurality of wires having a distal end and a proximal end;
wherein the distal end is connected to a camera board with a camera and the electrical cable extending proximally through the insertion tube;
wherein the plurality of wires at the distal end of the electric cable are soldered to the camera board;
wherein the plurality of wires at the proximal end are soldered to a flex circuit having a microconnector.
   61. A method of assembling the insertion tube assembly of clause 60, comprising:
passing the microconnector through a lumen of the insertion tube along a direction extending from a distal end of the insertion tube to a proximal end of the insertion tube.
   62. The method of clause 61, comprising connecting the microconnector to an electrical connector in a handle of the endoscope.

1. A medical device, comprising:
a first pulley associated with a first articulation wire of the medical device for deflection of a distal end of the medical device in a first plane upon rotation of the first pulley;
wherein the first pulley is rotatable no more than a first rotational distance relative to a rotational stop idler; and
wherein the rotational stop idler is rotatable no more than a second rotational distance relative to a housing of the endoscope; and
wherein the first pulley is rotatable relative to the housing no more than the sum of the first rotational distance and the second rotational distance.

2. The medical device of claim 1, wherein the rotational stop idler has a first side contacting the first pulley and a second side contact the housing.

3. The medical device of claim 2, wherein the first side includes a first groove and the second side includes a second groove;
wherein a boss on the first pulley is slidably received within the first groove of the rotational stop idler; and
wherein a boss on the housing is slidably received within the second groove of the rotational stop idler.

4. The medical device of claim 1, comprising:
a second pulley associated with a second articulation wire of the medical device for deflection of the distal end of the medical device in a second plane upon rotation of the second pulley;
wherein the second pulley is rotatable no more than a third rotational distance relative to a second rotational stop idler;
wherein the second rotational stop idler is rotatable no more than a fourth rotational distance relative to the housing of the endoscope; and
wherein the second pulley is rotatable relative to the housing no more than the sum of the third rotational distance and the fourth rotational distance.

5. The medical device of claim 4, wherein the second plane is orthogonal to the first plane.

6. The medical device of claim 4, wherein the second rotational stop idler has a first side contacting the second pulley and a second side contacting the housing.

7. The medical device of claim 6, wherein the first side of the second rotational stop idler includes a first groove and the second side includes a second groove;
wherein a boss on the second pulley is slidably received within the first groove of the second rotational stop idler; and
wherein a boss on the housing is slidably received within the second groove of the second rotational stop idler.

8. The medical device of claim 1, wherein the housing is part of a single-use shaft assembly; and
wherein the single-use shaft assembly is selectively attachable and detachable to a reusable hand-piece assembly having a control knob and shaft for rotating the first pulley.

9. The medical device of claim 1, wherein the medical device is an endoscope.

10. A medical device, comprising:
a reusable hand-piece assembly and a single-use shaft assembly;
the reusable hand-piece assembly selectively attachable to and detachable from the single-use shaft assembly; and
the reusable hand-piece assembly including a housing and an articulation and brake assembly, wherein the articulation and brake assembly includes a first articulation control knob, a second articulation control knob, and a first articulation brake knob; and
wherein the first articulation control knob, the second articulation control knob, and/or the first articulation brake knob are selectively attachable and detachable from the reusable hand-piece assembly.

11. The medical device of claim 10, wherein the first articulation control knob, the second articulation control knob, and/or the first articulation brake knob are secured to the reusable hand-piece assembly only by friction fit.

12. The medical device of claim 10, wherein the articulation and brake assembly is secured to the housing of the reusable hand-piece assembly by a retention clip.

13. The medical device of claim 12, wherein the retention clip is coupled to an up-down articulation hub to secure the articulation knob and brake assembly to the housing of the reusable hand-piece assembly.

14. An endoscope, comprising:
a first articulation brake configurable from a disengaged position that permits rotation of a first articulation control knob relative to a housing of the endoscope to an engaged position that resists rotation of the first articulation control knob relative to the housing;
wherein rotation of the first articulation control knob relative to the housing rotates a first articulation control shaft around a first axis;
wherein the articulation brake includes braking surfaces at a non-orthogonal angle relative to the first axis;
wherein when the first articulation brake is configured in the engaged configuration the braking surfaces are forced towards one another with a normal force having a first magnitude; and
wherein when the first articulation brake is configured in the disengaged position the braking surface are either separated or contacting one another with a normal force having a magnitude less than said first magnitude.

15. The endoscope of claim 14, wherein the housing is part of a reusable hand-piece assembly selectively attachable to and detachable from a single-use shaft assembly of the endoscope.
